(19) United States
(12) Patent Application Publication
(10) Pub. No.: US 2024/0239798 A1
(43) Pub. Date: Jul. 18, 2024

(54) SALTS AND POLYMORPHS OF MITRAGYNINE AND 3-DEUTEROMITRAGYNINE

(71) Applicant: KURES, INC., New York, NY (US)

(72) Inventor: Andrew KRUEGEL, Secaucus, NJ (US)

(73) Assignee: KURES, INC., New York, NY (US)

(21) Appl. No.: 18/293,615

(22) PCT Filed: May 6, 2022
(86) PCT No.: PCT/IB2022/054187

§ 371 Date: Jan. 30, 2024

Related U.S. Application Data
(60) Provisional application No. 63/185,326, filed on May 6, 2021.

(51) Int. Cl. C07D 471/14; A61K 31/4375

(52) U.S. Cl. CPC C07D 471/14; A61K 31/4375

(57) ABSTRACT

The present disclosure provides: one or more salts or crystalline forms of 3-deuteromitragynine of Formula I; and one or more salts or crystalline forms of mitragynine of Formula II. Pharmaceutical compositions comprising salts and crystalline forms of Formulae I and II and methods of treatment are also disclosed. Methods of preparing and purifying the salts of Formulae I and II are further disclosed.

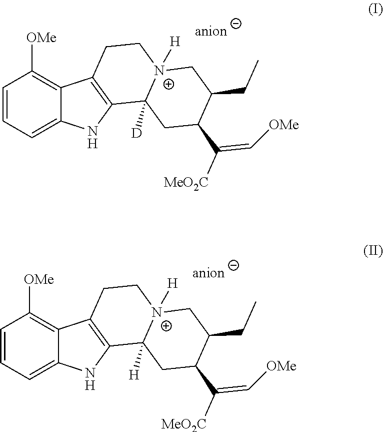 [SMILES: [2H][C@@]12C[C@H](/C(=C\OC)C(=O)OC)[C@H](CC)C[NH+]1CCc1c2[nH]c2cccc(OC)c12 ; CC[C@@H]1C[NH+]2CCc3c([nH]c4cccc(OC)c34)[C@@H]2C[C@@H]1/C(=C\OC)C(=O)OC]

### CROSS-REFERENCE TO RELATED APPLICATIONS

The present application claims the benefit of priority of U.S. Provisional Application Ser. No. 63/185,326, filed on May 6, 2021, the disclosure of which is incorporated herein by reference in their entireties for all purposes.

### BACKGROUND OF THE INVENTION

Mitragynine is a psychoactive compound derived from the leaves of the Southeast Asian plant Mitragyna speciosa, of the Rubiaceae (coffee family). Mitragynine is known to have strong analgesic effects, so it has been suggested as a useful compound in the treatment of pain and opioid addiction (as a replacement therapy).

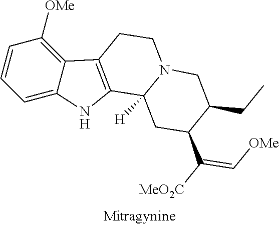 [SMILES: CC[C@@H]1CN2CCc3c([nH]c4cccc(OC)c34)[C@@H]2C[C@@H]1/C(=C\OC)C(=O)OC]

The structure of mitragynine in free base form is depicted above. Mitragynine is insoluble in water but soluble in conventional organic solvents, including acetone, acetic acid, alcohols, chloroform and diethyl ether. Mitragynine distils at 230-240° C. at 5 mmHg. It forms white, amorphous crystals that melt at 102-106° C. The melting point of mitragynine hydrochloride is 243° C.; the picrate melts at 223-224° C. and the acetate at 142° C. (https://www.emcdda.europa.eu/publications/drug-profiles/kratom_en)

The above mentioned mitragynine hydrochloride salt is known to form a gel in aqueous solution, which makes it difficult to formulate as a solution for injectable use as described by Ellen Field in J. Chem. Soc., Trans., 1921, 119, 887-891; XCVIII Murrayanine and Mitraversine, Two New Alkaloids from Species of Mitragyne.

Mitragynine isolated from natural sources is frequently contaminated with the closely related alkaloid corynantheidine (CR, pictured below), which is very difficult to remove by known methods.

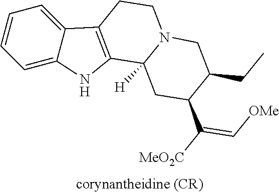 [SMILES: CC[C@@H]1CN2CCc3c([nH]c4ccccc34)[C@@H]2C[C@@H]1/C(=C\OC)C(=O)OC]

Recently, 3-deuteromitragynine (3-DM), pictured below, has been found to be a useful derivative of mitragynine as described in PCT/US2020/015898, published as WO2020160280A1, which is hereby incorporated by reference in its entirety for all purposes.

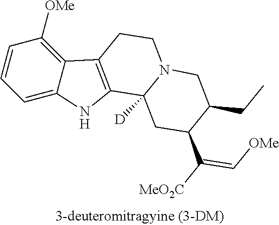 [SMILES: [2H][C@@]12C[C@H](/C(=C\OC)C(=O)OC)[C@H](CC)CN1CCc1c2[nH]c2cccc(OC)c12]

This compound has potential therapeutic properties similar to mitragynine and since it is a deuterated derivative, its physicochemical properties are also very similar. For example, its hydrochloride salt also forms a gel in aqueous solution. Further, 3-DM may be contaminated with 3-deuterocorynantheidine (3-DCR).

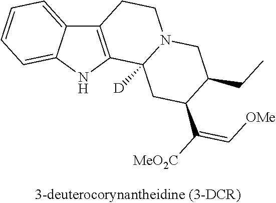 [SMILES: [2H][C@@]12C[C@H](/C(=C\OC)C(=O)OC)[C@H](CC)CN1CCc1c2[nH]c2ccccc12]

There is an ongoing need for alternative salts and crystalline forms of mitragynine and 3-DM that better exclude impurities, reduce gel formation, and offer different physicochemical properties, such as pKa, solubility, and melting point.

### SUMMARY OF THE INVENTION

In one aspect, the present disclosure provides a salt of 3-deuteromitragynine of Formula I:

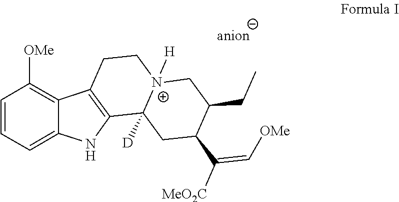 [SMILES: [2H][C@@]12C[C@H](/C(=C\OC)C(=O)OC)[C@H](CC)C[NH+]1CCc1c2[nH]c2cccc(OC)c12]

wherein the anion is glycolate, L-lactate, succinate, fumarate, or mesylate.

In some embodiments, the anion is glycolate. In some embodiments, the salt of 3-deuteromitragynine of Formula I is glycolate Type A, glycolate Type B, glycolate Type C, glycolate Type D, glycolate Type E, or glycolate Type F.

In some embodiments, the salt of 3-deuteromitragynine of Formula I is glycolate Type A. In some embodiments, the glycolate Type A is characterized by peaks in an X-ray diffraction (XRPD) pattern at 7.1±0.2, 10.1±0.2, and 11.2±0.2° 2θ.

In some embodiments, the salt of 3-deuteromitragynine of Formula I is glycolate Type B. In some embodiments, the glycolate Type B is characterized by peaks in an XRPD pattern at 5.3±0.2, 5.7±0.2, and 7.5±0.2° 2θ.

In some embodiments, the salt of 3-deuteromitragynine of Formula I is glycolate Type C. In some embodiments, the glycolate Type C is characterized by peaks in an XRPD pattern at 6.0±0.2, 7.4±0.2, and 24.2±0.2° 2θ.

In some embodiments, the salt of 3-deuteromitragynine of Formula I is glycolate Type D. In some embodiments, the glycolate Type D is characterized by peaks in an XRPD pattern at 5.3±0.2, 6.8±0.2, and 9.0±0.2° 2θ.

In some embodiments, the salt of 3-deuteromitragynine of Formula I is glycolate Type E. In some embodiments, the glycolate Type E is characterized by peaks in an XRPD pattern at 5.1±0.2, 7.8±0.2, and 8.8±0.2° 2θ.

In some embodiments, the salt of 3-deuteromitragynine of Formula I is glycolate Type F. In some embodiments, the glycolate Type F is characterized by peaks in an XRPD pattern at 5.9±0.2, 6.4±0.2, and 7.2±0.2° 2θ.

In some embodiments, the salt of 3-deuteromitragynine of Formula I is L-lactate. In some embodiments, the L-lactate salt is characterized by peaks in an XRPD pattern at 6.9±0.2, 10.0±0.2, and 11.0±0.2° 2θ.

In some embodiments, the salt of 3-deuteromitragynine of Formula I is succinate. In some embodiments, the succinate salt is characterized by peaks in an XRPD pattern at 8.5±0.2, 17.6±0.2, and 19.3±0.2° 2θ.

In some embodiments, the salt of 3-deuteromitragynine of Formula I is fumarate. In some embodiments, the fumarate salt is characterized by peaks in an XRPD pattern at 8.4±0.2, 17.5±0.2, and 19.2±0.2° 2θ.

In some embodiments, the salt of 3-deuteromitragynine of Formula I is mesylate. In some embodiments, the mesylate salt is characterized by peaks in an XRPD pattern at 6.7±0.2, 16.7±0.2, and 17.3±0.2° 2θ.

In some embodiments, the present disclosure provides pharmaceutical compositions comprising a salt of 3-deuteromitragynine of Formula I as described herein.

In one aspect, the present disclosure provides a salt of mitragynine of Formula II

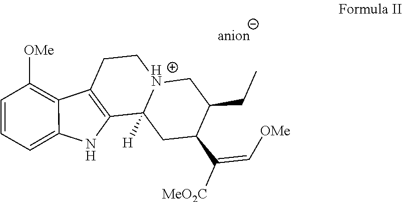

wherein the anion is glycolate, L-lactate, succinate, fumarate, or mesylate.

In some embodiments, the salt of mitragynine of Formula II is glycolate. In some embodiments, the glycolate salt is characterized by peaks in an XRPD pattern at 7.1±0.2, 10.2±0.2, and 11.3±0.2° 2θ.

In some embodiments, the salt of mitragynine of Formula II is L-lactate. In some embodiments, the L-lactate salt is characterized by peaks in an XRPD pattern at 7.0±0.2, 10.1±0.2, and 11.2±0.2° 2θ.

In some embodiments, the salt of mitragynine of Formula II is succinate. In some embodiments, the succinate salt is characterized by peaks in an XRPD pattern at 8.5±0.2, 17.6±0.2, and 19.3±0.2° 2θ.

In some embodiments, the salt of mitragynine of Formula II is fumarate. In some embodiments, the fumarate salt is characterized by peaks in an XRPD pattern at 8.3±0.2, 19.1±0.2, and 19.2±0.2° 2θ.

In some embodiments, the salt of mitragynine of Formula II is mesylate. In some embodiments, the mesylate salt is characterized by peaks in an XRPD pattern at 6.7±0.2, 16.7±0.2, and 17.4±0.2° 2θ.

In some embodiments, the present disclosure provides pharmaceutical compositions comprising a salt of mitragynine of Formula II as described herein.

In one aspect, the present disclosure provides a method of treating a subject afflicted with acute pain, chronic pain, a depressive disorder, a mood disorder, an anxiety disorder, borderline personality disorder, a substance use disorder, opioid use disorder, opioid withdrawal symptoms, alcohol use disorder, or alcohol withdrawal disorder, comprising administering an effective amount of a salt of 3-deuteromitragynine of Formula I or a salt of mitragynine of Formula II as described herein.

In some embodiments, present disclosure provides a method of treating opioid use disorder. In some embodiments, present disclosure provides a method of treating opioid withdrawal.

### Definitions

In each instance herein, in descriptions, embodiments, and examples of the present disclosure, the terms “comprising”, “including”, etc., are to be read expansively, without limitation. Thus, unless the context clearly requires otherwise, throughout the description and the claims, the words “comprise”, “comprising”, and the like are to be construed in an inclusive sense as opposed to an exclusive sense, that is to say in the sense of “including but not limited to”.

In the present description, the articles “a” and “an” are used to refer to one or to more than one (i.e., to at least one) of the grammatical object of the article. By way of example, “an element” can be taken to mean one element or more than one element.

Throughout this description, the term “about” is used to indicate that a value includes the standard deviation of error for the method being employed to determine the value, for example, dosage levels, as described in detail herein. In particular, the term “about” encompasses a 10% to 15% deviation (positive and negative) in the stated value or range, particularly 10% deviation (positive and negative) in the stated value or range.

The present disclosure is also intended to include all isotopes of atoms occurring on the salt(s) disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any salt(s) containing ¹³C or ¹⁴C may specifically have the structure of any of the salt(s) disclosed herein.

It will also be noted that any notation of a hydrogen in structures throughout this application, when used without further notation, are intended to represent all isotopes of hydrogen, such as ¹H, ²H, or ³H. Furthermore, any salt(s) containing ²H or ³H may specifically have the structure of any of the salt(s) disclosed herein.

Isotopically-labeled salt(s) can generally be prepared by conventional techniques known to those skilled in the art using appropriate isotopically-labeled reagents in place of the non-labeled reagents employed.

All references, including patents and patent applications, cited in this specification are hereby incorporated by reference. No admission is made that any reference constitutes prior art. Nor does discussion of any reference constitute an admission that such reference forms part of the common general knowledge in the art, in any country.

### BRIEF DESCRIPTION OF THE FIGURES

FIG. 1 shows an XRPD pattern of amorphous 3-DM free base starting material (810397-01-A).

FIG. 2 shows TGA/mDSC curves of amorphous 3-DM free base starting material (810397-01-A).

FIG. 3 shows an ¹H NMR spectrum of amorphous 3-DM free base starting material (810397-01-A).

FIG. 4 shows an XRPD pattern of Fumarate Type A 3-DM Salt.

FIG. 5 shows an XRPD pattern of L-Lactate Type A 3-DM Salt.

FIG. 6 shows an XRPD pattern of Glycolate Type A 3-DM Salt.

FIG. 7 shows an XRPD pattern of Succinate Type A 3-DM Salt.

FIG. 8 shows an XRPD pattern of Mesylate Type A 3-DM Salt.

FIG. 9 shows an XRPD pattern of HCl Type A 3-DM Salt (810397-04-B1).

FIG. 10 shows an XRPD pattern of HCl Type B 3-DM Salt (810397-04-D1).

FIG. 11 XRPD pattern of Phosphate Type A 3-DM Salt (810397-04-D3).

FIG. 12 shows an XRPD pattern of Maleate Type A 3-DM Salt (810397-04-C4).

FIG. 13 shows an XRPD pattern of L-Tartrate Type A 3-DM Salt (810397-04-B5).

FIG. 14 shows an XRPD pattern of Glycolate Type B 3-DM Salt (810397-04-C8).

FIG. 15 shows an XRPD pattern of Adipate Type A 3-DM Salt (810397-04-A12).

FIG. 16 shows an XRPD pattern of Adipate Type B 3-DM Salt (810397-04-C12).

FIG. 17 shows an XRPD pattern of Acetate Type A 3-DM Salt (810397-04-D13).

FIG. 18 shows an XRPD pattern of Malonate Type A 3-DM Salt (810397-04-B17).

FIG. 19 shows an XRPD pattern of Malonate Type B 3-DM Salt (810397-04-D17)

FIG. 20 shows an XRPD pattern of Gentisate Type A 3-DM Salt (810397-04-B18).

FIG. 21 shows an XRPD pattern of Gentisate Type B 3-DM Salt (810397-04-C18).

FIG. 22 shows an XRPD pattern of Edisylate Type A 3-DM Salt (810397-04-A19).

FIG. 23 shows an XRPD pattern of Edisylate Type B 3-DM Salt (810397-04-C19).

FIG. 24 shows an XRPD pattern of Benzoate Type A 3-DM Salt (810397-04-D20).

FIG. 25 shows an XRPD pattern of Esylate Type A 3-DM Salt (810397-04-C21).

FIG. 26 shows an XRPD pattern of Besylate Type A 3-DM Salt (810397-04-D23).

FIG. 27 shows an XRPD pattern of Tosylate Type A 3-DM Salt (810397-04-B24).

FIG. 28 shows an XRPD pattern of Oxalate Type A 3-DM Salt (810397-04-A25).

FIG. 29 shows an XRPD pattern of Oxalate Type B 3-DM Salt (810397-04-B25).

FIG. 30 shows an XRPD overlay of 3-DM Glycolate crystal forms.

FIG. 31 shows a polarized light microscopy (PLM) image of 3-DM Glycolate Type A starting material used in crystallization process development experiments (Examples 18-21).

FIG. 32 shows an XRPD pattern of 3-DM Free Base Type A (810081-04-C1_dry).

FIG. 33 shows a polarized light microscopy (PLM) image of 3-DM Glycolate Type A prepared at 5-g scale (810082-25-B).

FIG. 34 shows an XRPD pattern of amorphous mitragynine free base starting material (810080-01-A).

FIG. 35 shows TGA/mDSC curves of amorphous mitragynine free base starting material (810080-01-A).

FIG. 36 shows an ¹H NMR spectrum of amorphous mitragynine free base starting material (810080-01-A).

FIG. 37 shows an XRPD pattern of crude alkaloid extract starting material (810080-01-B).

FIG. 38 shows a TGA curve of crude alkaloid extract starting material (810080-01-B).

FIG. 39 shows an XRPD pattern of Fumarate Type A Mitragynine Salt.

FIG. 40 shows an XRPD pattern of L-Lactate Type A Mitragynine Salt.

FIG. 41 shows an XRPD pattern of Glycolate Type A Mitragynine Salt.

FIG. 42 shows an XRPD pattern of Succinate Type A Mitragynine Salt.

FIG. 43 shows an XRPD pattern of Mesylate Type A Mitragynine Salt.

### DETAILED DESCRIPTION

The following description sets forth numerous exemplary configurations, parameters, and the like. It should be recognized, however, that such description is not intended as a limitation on the scope of the present disclosure but is instead provided as a description of exemplary embodiments.

### Salts of 3-Deuteromitragynine

In one aspect the present disclosure provides one or more salts of 3-deuteromitragynine of Formula I:

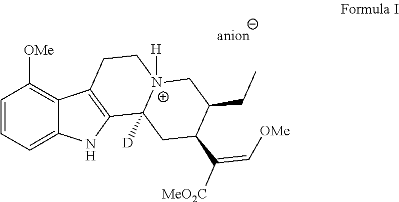 [SMILES: [2H][C@@]12C[C@H](/C(=C\OC)C(=O)OC)[C@H](CC)C[NH+]1CCc1c2[nH]c2cccc(OC)c12]

wherein the anion is glycolate, L-lactate, succinate, fumarate or mesylate.

In some embodiments, the anion is glycolate (i.e., the salt is a glycolate salt).

In some embodiments, the glycolate salt of 3-deuteromitragynine exhibits an XRPD spectrum with copper radiation having 2 theta peaks at approximately:

Pos. [°2θ]

7.06
10.12
11.25
15.97
17.99
18.11
19.49
19.69
20.34
20.88
22.58
25.16
27.60
±0.2 degrees 2 theta.

In some embodiments, the salt of 3-deuteromitragynine of Formula I is glycolate Type A, glycolate Type B, glycolate Type C, glycolate Type D, glycolate Type E, glycolate Type F, or combinations thereof.

In some embodiments, the salt of 3-DM is glycolate Type A.

In some embodiments, the glycolate Type A is characterized by peaks in an X-ray diffraction (XRPD) pattern at 7.1±0.2, 10.1±0.2, and 11.2±0.2° 2θ. In some embodiments, the glycolate Type A is further characterized by at least one XRPD peak selected from 16.0±0.2, 18.0±0.2, 19.5±0.2, 20.9±0.2, 22.6±0.2, and 25.2±0.2° 2θ.

In some embodiments, the glycolate Type A is characterized by peaks in an XRPD pattern at 7.1±0.2, 10.1±0.2, 13.2±0.2, 16.0±0.2, 18.0±0.2, 19.5±0.2, 20.9±0.2, 22.6±0.2, and 25.2±0.2° 2θ. In some embodiments, the glycolate Type A is further characterized by at least one XRPD peak selected from 13.2±0.2, 14.1±0.2, 15.0±0.2, 18.5±0.2, 19.2±0.2, 19.7±0.2, 20.3±0.2, 23.7±0.2, 240±0.2, 27.6±0.2, 29.5±0.2, 30.1±0.2, 31.6±0.2, and 34.1±0.2° 2θ.

In some embodiments, the glycolate Type A is characterized by peaks in an XRPD pattern at 7.1±0.2, 10.1±0.2, and 11.2±0.2° 2θ and at least one XRPD peak selected from 16.0±0.2, 18.0±0.2, 19.5±0.2, 19.7±0.2, 20.3±0.2, 20.9±0.2, 22.6±0.2, 25.2±0.2 and 27.6±0.2° 2θ.

In some embodiments, the glycolate Type A is characterized by peaks in an XRPD pattern at 7.1±0.2, 10.1±0.2, 11.2±0.2, 13.2±0.2, 14.1±0.2, 15.1±0.2, 16.0±0.2, 18.0±0.2, 18.5±0.2, 19.2±0.2, 19.5±0.2, 19.7±0.2, 20.3±0.2, 20.9±0.2, 22.6±0.2, 23.7±0.2, 24.0±0.2, 25.2±0.2, 27.6±0.2, 29.5±0.2, 30.2±0.2, 31.6±0.2, and 34.1±0.2° 2θ.

In some embodiments, the glycolate Type A exhibits a weight loss of about 1% up to a temperature of about 150° C. as measured by thermogravimetric (TGA) analysis.

In some embodiments, the glycolate Type A exhibits a Differential Scanning calorimetry (DSC) thermogram comprising an endotherm peak at about 222±2.5° C.

In some embodiments, a glycolate Type A salt of 3-DM is provided exhibiting an XRPD spectrum with copper radiation having 2 theta peaks at about:

Pos. [°2θ]

7.06
10.11
11.24
15.96
18.01
19.49
19.70
20.34
20.88
22.57
25.15
27.62
±0.2 degrees 2 theta.

In some embodiments, the salt of 3-DM is glycolate Type B.

In some embodiments, the glycolate Type B is characterized by peaks in an XRPD pattern at 5.3±0.2, 5.7±0.2, and 7.5±0.2° 2θ. In some embodiments, the glycolate Type B is further characterized by at least one XRPD peak selected from 6.8±0.2, 10.8±0.2, 13.7±0.2, 19.9±0.2, 22.7±0.2, and 27.4±0.2° 2θ.

In some embodiments, the glycolate Type B is characterized by peaks in an XRPD pattern at 5.3±0.2, 5.7±0.2, 6.8±0.2, 7.5±0.2, 10.8±0.2, 13.7±0.2, 19.9±0.2, 22.7±0.2, and 27.4±0.2° 2θ. In some embodiments, the glycolate Type B is further characterized by at least one XRPD peak selected from 9.0±0.2, 14.7±0.2, 17.4±0.2, 21.2±0.2, 24.1±0.2, and 25.4±0.2° 2θ.

In some embodiments, the glycolate Type B is characterized by peaks in an XRPD pattern at 5.3±0.2, 5.7±0.2, and 7.5±0.2° 2θ and at least one XRPD peak selected from 10.8±0.2, 13.7±0.2, 19.9±0.2, 21.2±0.2, 22.7±0.2, and 24.0±0.2° 2θ.

In some embodiments, the glycolate Type B is characterized by peaks in an XRPD pattern at 5.3±0.2, 5.7±0.2, 6.8±0.2, 7.5±0.2, 9.0±0.2, 10.8±0.2, 13.7±0.2, 14.7±0.2, 17.4±0.2, 19.9±0.2, 21.2±0.2, 22.7±0.2, 24.0±0.2, 25.4±0.2, and 27.4±0.2° 2θ.

In some embodiments, the glycolate Type B exhibits a weight loss of about 4% up to a temperature of about 120° C. as measured by TGA analysis. In some embodiments, the glycolate Type B further exhibits a weight loss of about 8% between a temperature ranging from about 120° C. to about 160° C. as measured by TGA analysis.

In some embodiments, the glycolate Type B exhibits a DSC thermogram comprising an endothermic peak at about 147±2.5° C. and 223±2.5° C.

In some embodiments, a glycolate Type B salt of 3-DM is provided exhibiting an XRPD spectrum with copper radiation having 2 theta peaks at about:

Pos. [°2θ]

5.29
5.68
7.45
10.78
13.65
19.86
21.22
22.72
24.05
±0.2 degrees 2 theta.

In some embodiments, the salt of 3-DM is glycolate Type C.

In some embodiments, the glycolate Type C is characterized by peaks in an XRPD pattern at 6.0±0.2, 7.4±0.2, and 24.2±0.2° 2θ. In some embodiments, the glycolate Type C is further characterized by at least one XRPD peak selected from 14.2±0.2, 16.3±0.2, 18.1±0.2, 20.1±0.2, 26.2±0.2, and 27.6±0.2° 2θ.

In some embodiments, the glycolate Type C is characterized by peaks in an XRPD pattern at 6.0±0.2, 7.4±0.2, 14.2±0.2, 16.3±0.2, 18.1±0.2, 20.1±0.2, 24.2±0.2, 26.2±0.2, and 27.6±0.2° 2θ. In some embodiments, the glycolate Type C is further characterized by at least one XRPD peak selected from 10.6±0.2, 11.0±0.2, 11.4±0.2, 12.8±0.2, 13.7±0.2, 15.7±0.2, 18.8±0.2, 21.2±0.2, 21.5±0.2, 22.6±0.2, 22.9±0.2, 25.1±0.2, 28.6±0.2, 29.4±0.2, 31.6±0.2, 33.7±0.2, 35.2±0.2, and 38.3±0.2° 2θ.

In some embodiments, the glycolate Type C is characterized by peaks in an XRPD pattern at 6.0±0.2, 7.4±0.2, and 24.2±0.2° 2θ and at least one XRPD peak at 11.0±0.2, 11.4±0.2, 13.7±0.2, 14.2±0.2, 16.3±0.2, 18.1±0.2, 18.8±0.2, 20.1±0.2, 21.5±0.2, 22.9±0.2, and 26.2±0.2° 2θ.

In some embodiments, the glycolate Type C is characterized by peaks in an XRPD pattern at 6.0±0.2, 7.4±0.2, 10.6±0.2, 11.0±0.2, 11.4±0.2, 12.8±0.2, 13.7±0.2, 14.2±0.2, 15.7±0.2, 16.3±0.2, 17.6±0.2, 18.1±0.2, 18.8±0.2, 20.1±0.2, 21.2±0.2, 21.5±0.2, 22.6±0.2, 22.9±0.2, 24.2±0.2, 25.2±0.2, 26.2±0.2, 27.6±0.2, 28.6±0.2, 29.4±0.2, 31.6±0.2, 33.7±0.2, 35.3±0.2, and 38.3±0.2° 2θ.

In some embodiments, the glycolate Type C exhibits a weight less of about 6% up to a temperature of about 150° C. as measured by TGA.

In some embodiments, the glycolate Type C exhibits a DSC thermogram comprising an endotherm peak at about 61±2.5° C., 141±2.5° C., and about 222±2.5° C.

In some embodiments, a glycolate Type C salt of 3-DM is provided exhibiting an XRPD spectrum with copper radiation having 2 theta peaks at about:

Pos. [°2θ]

6.03
7.43
10.99
11.39
13.65
14.22
16.30
18.07
18.84
20.08
21.54
22.87
24.16
26.22
±0.2 degrees 2 theta.

In some embodiments, the salt of 3-DM is glycolate Type D.

The salt of embodiment 32, wherein the glycolate Type D is characterized by peaks in an XRPD pattern at 5.3±0.2, 6.8±0.2, and 9.0±0.2° 2θ. In some embodiments, the glycolate Type D is further characterized by at least one XRPD peak selected from 11.0±0.2, 13.5±0.2, 17.3±0.2, 19.5±0.2, 20.1±0.2, and 21.3±0.2° 2θ.

In some embodiments, the glycolate Type D is characterized by peaks in an XRPD pattern at 5.3±0.2, 6.8±0.2, 9.0±0.2, 11.0±0.2, 13.5±0.2, 17.3±0.2, 19.5±0.2, 20.1±0.2, and 21.3±0.2° 2θ. In some embodiments, the glycolate Type D is further characterized by at least one XRPD peak selected from 10.1±0.2, 11.3±0.2, 13.9±0.2, 16.5±0.2, 17.0±0.2, 18.0±0.2, 22.9±0.2, 23.7±0.2, 25.5±0.2, and 27.3±0.2° 2θ.

In some embodiments, the glycolate Type D is characterized by peaks in an XRPD pattern at 5.3±0.2, 6.8±0.2, and 9.0±0.2° 2θ and at least one XRPD peak selected from 11.0±0.2, 11.3±0.2, 13.5±0.2, 13.9±0.2, 17.3±0.2, 19.5±0.2, 20.1±0.2, 21.3±0.2, and 23.7±0.2° 2θ.

In some embodiments, the glycolate Type D is characterized peaks in an XRPD pattern at 5.3±0.2, 6.8±0.2, 9.0±0.2, 10.1±0.2, 11.0±0.2, 11.3±0.2, 13.5±0.2, 13.9±0.2, 16.5±0.2, 17.0±0.2, 17.3±0.2, 18.0±0.2, 19.5±0.2, 20.1±0.2, 21.3±0.2, 22.9±0.2, 23.7±0.2, 25.5±0.2, and 27.3±0.2° 2θ.

In some embodiments, the glycolate Type D exhibits a weight less of about 3% up to a temperature of about 100° C. as measured by TGA.

In some embodiments, the glycolate Type D exhibits a DSC thermogram comprising an endotherm peak at about 63±2.5° C., about 210±2.5° C., and about 123±2.5° C.

In some embodiments, a glycolate Type D salt of 3-DM is provided exhibiting an XRPD spectrum with copper radiation having 2 theta peaks at about:

Pos. [°2θ]

5.33
6.82
9.04
11.02
11.33
13.53
13.85
17.28
19.50
20.08
21.28
23.71
±0.2 degrees 2 theta.

In some embodiments, the salt of 3-DM is glycolate Type E.

In some embodiments, the glycolate Type E is characterized by peaks in an XRPD pattern at 5.1±0.2, 7.8±0.2, and 8.8±0.2° 2θ. In some embodiments, the glycolate Type E is further characterized by at least one XRPD peak selected from 11.0±0.2, 12.0±0.2, 15.1±0.2, 16.8±0.2, 19.1±0.2, and 21.0±0.2° 2θ.

In some embodiments, the glycolate Type E is characterized by peaks in an XRPD pattern at 5.1±0.2, 7.8±0.2, 8.8±0.2, 11.0±0.2, 12.0±0.2, 15.1±0.2, 16.8±0.2, 19.1±0.2, and 21.0±0.2° 2θ. In some embodiments, the glycolate Type E is further characterized by at least one XRPD peak selected from 18.4±0.2 and 22.3±0.2° 2θ.

In some embodiments, the glycolate Type E is characterized by peaks in an XRPD pattern at 5.1±0.2, 7.8±0.2, and 8.8±0.2° 2θ and at least one peak selected from 11.0±0.2, 12.0±0.2, 16.8±0.2, 18.4±0.2, 19.1±0.2, 20.9±0.2, and 22.3±0.2° 2θ.

In some embodiments, the glycolate Type E is characterized by peaks in an XRPD pattern at 5.1±0.2, 7.8±0.2, 8.8±0.2, 11.0±0.2, 12.0±0.2, 15.1±0.2, 16.8±0.2, 18.4±0.2, 19.1±0.2, 20.9±0.2, and 22.3±0.2° 2θ.

In some embodiments, a glycolate Type E salt of 3-DM is provided exhibiting an XRPD spectrum with copper radiation having 2 theta peaks at about:

Pos. [°2θ]

5.10
7.81
8.82
10.97
12.02
16.79
18.40
19.08
20.91
22.25
±0.2 degrees 2 theta.

In some embodiments, the salt of 3-DM is glycolate Type F.

In some embodiments, the glycolate Type F is characterized by peaks in an XRPD pattern at 5.9±0.2, 6.4±0.2, and 7.2±0.2° 2θ. In some embodiments, the glycolate Type F is further characterized by at least one XRPD peak selected from 13.4±0.2, 14.1±0.2, 18.4±0.2, 19.8±0.2, 24.7±0.2, and 23.9±0.2° 2θ.

In some embodiments, the glycolate Type F is characterized by peaks in an XRPD pattern at 5.9±0.2, 6.4±0.2, 7.2±0.2, 13.4±0.2, 14.1±0.2, 18.4±0.2, 19.8±0.2, 24.7±0.2, and 23.9±0.2° 2θ. In some embodiments, the glycolate Type F is further characterized by at least one XRPD peak selected from 10.3±0.2, 10.8±0.2, 11.2±0.2, 12.3±0.2, 16.1±0.2, 16.8±0.2, 17.2±0.2, 17.8±0.2, 20.6±0.2, 21.4±0.2, 22.1±0.2, 24.4±0.2, 26.0±0.2, 26.4±0.2, and 27.2±0.2° 2θ.

In some embodiments, the glycolate Type F is characterized by peaks in an XRPD pattern at 5.9±0.2, 6.4±0.2, and 7.2±0.2° 2θ and at least one XRPD peak selected from 10.8±0.2, 11.2±0.2, 13.4±0.2, 14.1±0.2, 16.11±0.2, 17.8±0.2, 18.4±0.2, 19.8±0.2, 21.4±0.2, 22.1±0.2, 23.7±0.2, 23.9±0.2 and 24.4±0.2° 2θ.

In some embodiments, the glycolate Type F is characterized by peaks in an XRPD pattern at 5.9±0.2, 6.4±0.2, 7.2±0.2, 10.3±0.2, 10.8±0.2, 11.2±0.2, 12.3±0.2, 13.4±0.2, 14.1±0.2, 16.1±0.2, 16.8±0.2, 17.2±0.2, 17.8±0.2, 18.4±0.2, 19.8±0.2, 20.6±0.2, 21.4±0.2, 22.1±0.2, 23.7±0.2, 23.9±0.2, 24.4±0.2, 26.0±0.2, 26.4±0.2, and 27.2±0.2° 2θ.

In some embodiments, a glycolate Type F salt of 3-DM is provided exhibiting an XRPD spectrum with copper radiation having 2 theta peaks at about:

Pos. [°2θ]

5.86
6.39
7.21
10.83
11.22
13.41
14.05
16.13
17.76
18.42
19.81
21.36
22.07
23.68
23.87
24.38
±0.2 degrees 2 theta.

In some embodiments, the anion is L-lactate (i.e., the salt is a L-lactate salt).

In some embodiments, the L-lactate salt is characterized by peaks in an XRPD pattern at 6.9±0.2, 10.0±0.2, and 11.0±0.2° 2θ. In some embodiments, the L-lactate salt is further characterized by at least one XRPD peak selected from 15.7±0.2, 20.6±0.2, 22.3±0.2, and 24.8±0.2° 2θ.

In some embodiments, the L-lactate salt is characterized by peaks in an XRPD pattern at 6.9±0.2, 10.0±0.2, 11.0±0.2, 15.7±0.2, 20.6±0.2, 22.3±0.2, and 24.8±0.2° 2θ. In some embodiments, the L-lactate salt is further characterized by at least one XRPD peak selected from 10.7±0.2, 13.0±0.2, 13.8±0.2, 17.7±0.2, 18.1±0.2, 18.8±0.2, 19.3±0.2, 19.8±0.2, 23.6±0.2, 24.4±0.2, 27.0±0.2, 28.0±0.2, 29.3±0.2, 31.2±0.2, 33.8±0.2, 35.6±0.2° 2θ.

In some embodiments, the L-lactate salt is characterized by peaks in an XRPD pattern at 6.9±0.2, 10.0±0.2, 1.07±0.2, 11.0±0.2, 13.0±0.2, 13.8±0.2, 15.8±0.2, 17.7±0.2, 18.1±0.2, 18.8±0.2, 19.3±0.2, 19.8±0.2, 20.6±0.2, 22.3±0.2, 23.6±0.2, 24.4±0.2, 24.8±0.2, 27.0±0.2, 27.1±0.2, and 35.6±0.2° 2θ.

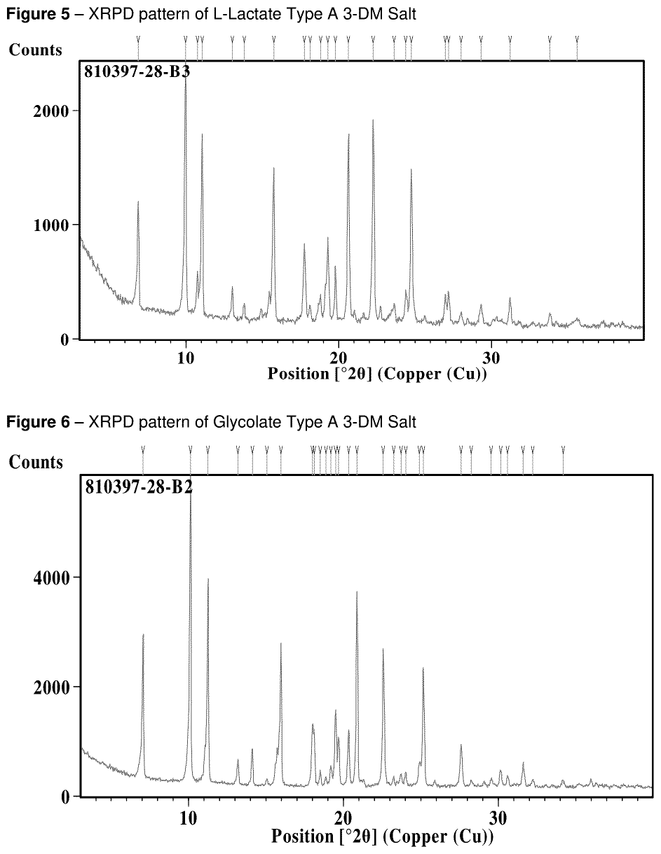

In some embodiments, the L-lactate salt is characterized by an XRPD pattern substantially similar to that shown in FIG. 5.

In some embodiments, the L-lactate salt exhibits a weight less of about 1% up to a temperature of about 150° C. as measured by TGA.

In some embodiments, the L-lactate salt exhibits a DSC thermogram comprising an endotherm peak at about 218±2.5° C.

In some embodiments, the anion is L-lactate and the 3-deuteromitragynine salt exhibits an XRPD spectrum with copper radiation having 2 theta peaks at approximately:

Pos. [°2θ]

6.86
9.96
10.73
11.05
15.74
19.77
20.63
22.26
24.75
±0.2 degrees 2 theta.

In some embodiments, the anion is succinate (i.e., the salt is a succinate salt).

In some embodiments, the succinate salt is characterized by peaks in an XRPD pattern at 8.5±0.2, 17.6±0.2, and 19.3±0.2° 2θ. In some embodiments, the succinate salt is further characterized by at least one XRPD peak selected 9.6±0.2, 21.7±0.2, 23.1±0.2, 25.5±0.2, and 25.9±0.2° 2θ.

In some embodiments, the succinate salt is characterized by peaks in an XRPD pattern at 8.5±0.2, 9.6±0.2, 17.6±0.2, 19.3±0.2, 21.7±0.2, 23.1±0.2, 25.5±0.2, and 25.9±0.2° 2θ. In some embodiments, the succinate salt is further characterized by at least one XRPD peak selected from 6.2±0.2, 10.1±0.2, 14.4±0.2, 15.7±0.2, 16.1±0.2, 16.9±0.2, 18.7±0.2, 21.2±0.2, 22.3±0.2, 23.7±0.2, 24.5±0.2, 25.0±0.2, 25.3±0.2, 27.0±0.2, 29.1±0.2, 30.5±0.2, 33.0±0.2, and 34.3±0.2° 2θ.

In some embodiments, the succinate salt is characterized by peaks in an XRPD pattern at 6.2±0.2, 8.5±0.2, 9.6±0.2, 10.0±0.2, 14.4±0.2, 15.7±0.2, 16.1±0.2, 16.9±0.2, 17.6±0.2, 18.7±0.2, 19.3±0.2, 21.2±0.2, 21.7±0.2, 22.3±0.2, 23.1±0.2, 23.7±0.2, 24.5±0.2, 25.0±0.2, 25.3±0.2, 25.5±0.2, 25.9±0.2, 27.0±0.2, 29.1±0.2, 30.5±0.2, 31.3±0.2, 33.0±0.2, and 34.0±0.2° 2θ.

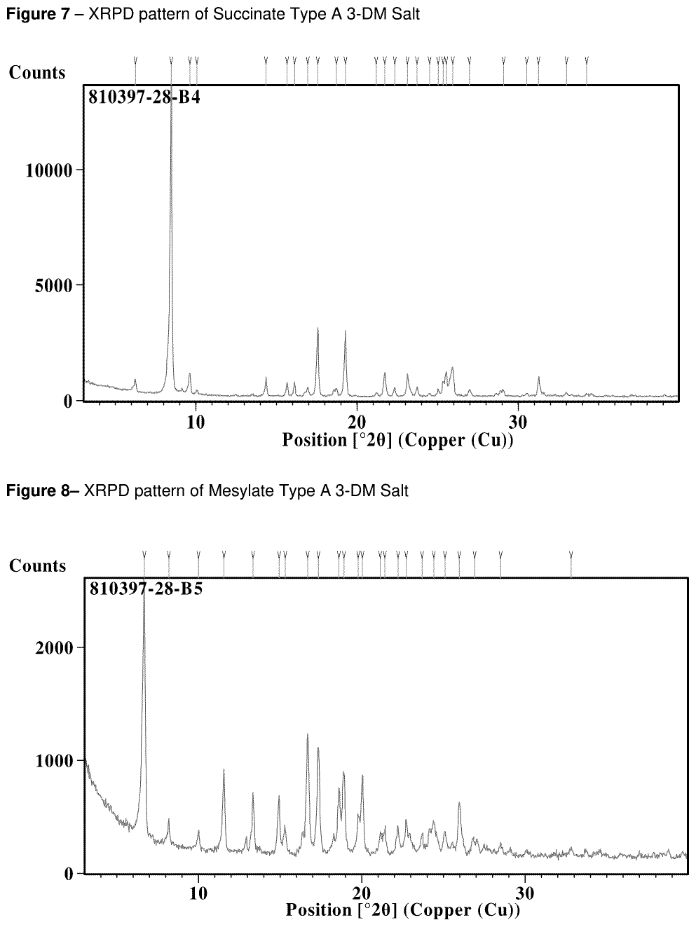

In some embodiments, the succinate salt is characterized by an XRPD pattern substantially similar to that shown in FIG. 7.

In some embodiments, the succinate salt exhibits a weight less of about 2% up to a temperature of about 150° C. as measured by TGA.

In some embodiments, the succinate salt exhibits a DSC thermogram comprising an endothermic peak at about 198±2.5° C. and about 202±2.5° C.

In some embodiments, the anion is succinate and the 3-deuteromitragynine salt exhibits an XRPD spectrum with copper radiation having 2 theta peaks at approximately:

Pos. [°2θ]

8.46
10.06
17.56
19.27
21.71
23.13
25.53
25.94
31.27
±0.2 degrees 2 theta.

In some embodiments, the anion is fumarate (i.e., the salt is a fumarate salt).

The salt of embodiment 73, wherein the fumarate salt is characterized by peaks in an XRPD pattern at 8.4±0.2, 17.5±0.2, and 19.2±0.2° 2θ. In some embodiments, the fumarate salt is further characterized by at least one XRPD peak selected from 9.6±0.2, 21.6±0.2, 25.4±0.2, 25.8±0.2, and 31.1±0.2° 2θ.

In some embodiments, the fumarate salt is characterized by peaks in an XRPD pattern at 8.4±0.2, 9.6±0.2, 17.5±0.2, 19.2±0.2, 21.6±0.2, 25.4±0.2, 25.8±0.2, and 31.1±0.2° 2θ. In some embodiments, the fumarate salt is further characterized by at least one XRPD peak selected from 13.4±0.2, 14.4±0.2, 15.6±0.2, 16.2±0.2, 16.9±0.2, 18.7±0.2, 22.4±0.2, 23.0±0.2, 23.4±0.2, 23.8±0.2, 27.0±0.2, 28.9±0.2, 32.8±0.2, 34.0±0.2, and 38.0±0.2° 2θ.

In some embodiments, the fumarate salt is characterized by peaks in an XRPD pattern at 8.4±0.2, 9.6±0.2, 13.4±0.2, 14.4±0.2, 15.6±0.2, 16.2±0.2, 16.9±0.2, 17.5±0.2, 18.7±0.2, 19.2±0.2, 21.6±0.2, 23.4±0.2, 23.0±0.2, 23.4±0.2, 23.8±0.2, 25.4±0.2, 25.8±0.2, 27.0±0.2, 28.9±0.2, 31.1±0.2, 32.8±0.2, 34.0±0.2, and 38.0±0.2° 2θ.

In some embodiments, the fumarate salt is characterized by an XRPD pattern substantially similar to that shown in FIG. 4.

In some embodiments, the fumarate salt exhibits a weight less of about 1% up to a temperature of about 150° C. as measured by TGA.

In some embodiments, the fumarate salt exhibits a DSC thermogram comprising an endothermic peak at about 255±2.5° C.

In some embodiments, the anion is fumarate and the 3-deuteromitragynine salt exhibits an XRPD spectrum with copper radiation having 2 theta peaks at approximately:

Pos. [°2θ]

8.41
9.58
17.46
19.20
23.03
25.36
25.77
31.11
±0.2 degrees 2 theta.

In some embodiments, the anion is mesylate (i.e., the salt is a mesylate salt).

In some embodiments, the mesylate salt is characterized by peaks in an XRPD pattern at 6.7±0.2, 16.7±0.2, and 17.3±0.2° 2θ. In some embodiments, the mesylate salt is further characterized by at least one XRPD peak selected from 11.6±0.2, 13.3±0.2, 18.6±0.2, 18.9±0.2, and 20.0±0.2° 2θ.

In some embodiments, the mesylate salt is characterized by peaks in an XRPD pattern at 6.7±0.2, 11.6±0.2, 13.3±0.2, 16.7±0.2, 17.3±0.2, 18.6±0.2, 18.9±0.2, and 20.0±0.2° 2θ. In some embodiments, the mesylate salt further characterized by at least one XRPD peak selected from 8.2±0.2, 10.0±0.2, 14.9±0.2, 15.3±0.2, 19.8±0.2, 21.1±0.2, 21.4±0.2, 22.2±0.2, 22.7±0.2, 23.7±0.2, 24.4±0.2, 25.1±0.2, 26.0±0.2, 26.9±0.2, 28.5±0.2, and 32.8±0.2° 2θ.

In some embodiments, the mesylate salt is characterized by peaks in an XRPD pattern at 6.7±0.2, 8.2±0.2, 10.0±0.2, 11.6±0.2, 13.3±0.2, 14.9±0.2, 15.3±0.2, 16.7±0.2, 17.3±0.2, 18.6±0.2, 18.9±0.2, 19.8±0.2, 20.0±0.2, 21.1±0.2, 21.4±0.2, 22.2±0.2, 22.7±0.2, 23.7±0.2, 24.4±0.2, 25.1±0.2, 26.0±0.2, 26.9±0.2, 28.5±0.2, and 32.8±0.2° 2θ.

In some embodiments, the mesylate salt is characterized by an XRPD pattern substantially similar to that shown in FIG. 8.

In some embodiments, the mesylate salt exhibits a weight less of about 1% up to a temperature of about 150° C. as measured by TGA.

In some embodiments, the mesylate salt exhibits a DSC thermogram comprising an endothermic peak at about 266±2.5° C.

In some embodiments, the anion is mesylate and the 3-deuteromitragynine salt exhibits an XRPD spectrum with copper radiation having 2 theta peaks at approximately:

Pos. [°2θ]

6.67
11.56
13.34
14.93
16.69
17.34
18.60
18.89
20.03
22.21
22.72
25.97
±0.2 degrees 2 theta.

### Salts of Mitragynine

In one aspect the present disclosure provides one or more salts of mitragynine of Formula II

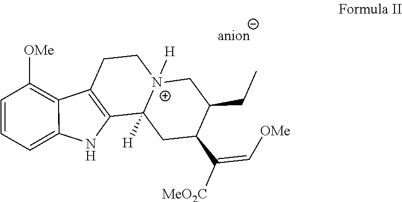 [SMILES: CC[C@@H]1C[NH+]2CCc3c([nH]c4cccc(OC)c34)[C@@H]2C[C@@H]1/C(=C\OC)C(=O)OC]

wherein the anion is glycolate, L-lactate, succinate, fumarate, or mesylate.

In some embodiments, the anion is glycolate (i.e., the salt is a glycolate salt).

The salt of embodiment 92, wherein the glycolate salt is characterized by peaks in an XRPD pattern at 7.1±0.2, 10.2±0.2, and 11.3±0.2° 2θ. In some embodiments, the glycolate salt is further characterized by at least one XRPD peak selected from 16.0±0.2, 18.0±0.2, 19.5±0.2, 20.9±0.2, 22.6±0.2, and 25.2±0.2° 2θ.

In some embodiments, the glycolate salt is characterized by peaks in an XRPD pattern at 7.1±0.2, 10.2±0.2, 11.3±0.2, 16.0±0.2, 18.0±0.2, 19.5±0.2, 20.9±0.2, 22.6±0.2, and 25.2±0.2° 2θ. In some embodiments, the glycolate salt is further characterized by at least one XRPD peak selected from 13.2±0.2, 14.1±0.2, 15.1±0.2, 15.7±0.2, 18.5±0.2, 18.9±0.2, 19.2±0.2, 19.7±0.2, 20.4±0.2, 23.3±0.2, 23.5±0.2, 23.4±0.2, 24.0±0.2, 24.9±0.2, 25.9±0.2, 27.6±0.2, 28.3±0.2, 29.1±0.2, 29.6±0.2, 30.2±0.2, 30.6±0.2, 32.7±0.2, 32.2±0.2, 34.3±0.2, 35.2±0.2, 36.0±0.2, and 36.6±0.2° 2θ.

In some embodiments, the glycolate salt is characterized by peaks in an XRPD pattern at 7.1±0.2, 10.2±0.2, 11.3±0.2, 13.2±0.2, 14.1±0.2, 15.1±0.2, 15.7±0.2, 16.0±0.2, 18.0±0.2, 18.5±0.2, 18.9±0.2, 19.2±0.2, 19.7±0.2, 20.4±0.2, 23.3±0.2, 23.5±0.2, 23.8±0.2, 24.1±0.2, 24.9±0.2, 25.9±0.2, 27.6±0.2, 28.3±0.2, 29.1±0.2, 29.6±0.2, 30.2±0.2, 30.6±0.2, 31.7±0.2, 32.2±0.2, 34.2±0.2, 35.2±0.2, 36.0±0.2, and 36.6±0.2° 2θ.

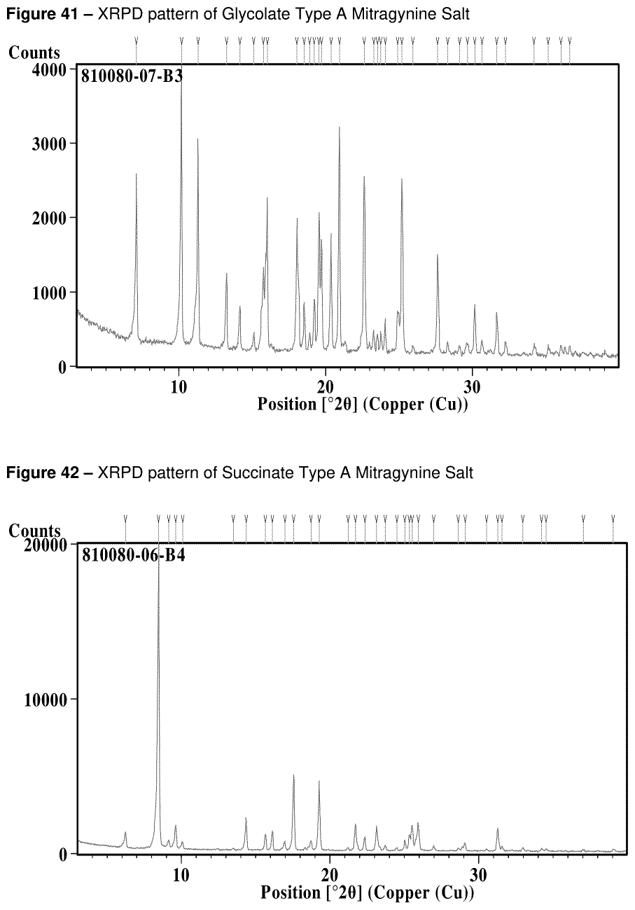

In some embodiments, the glycolate salt is characterized by an XRPD pattern substantially similar to that shown in FIG. 41.

In some embodiments, the glycolate salt exhibits a weight less of about 2% up to a temperature of about 150° C. as measured by TGA.

In some embodiments, the glycolate salt exhibits a DSC thermogram comprising an endothermic peak at about 220±2.5° C.

In some embodiments, the anion is glycolate and the mitragynine salt exhibits an XRPD spectrum with copper radiation having 2 theta peaks at about:

Pos. [°2θ]

7.09
10.16
11.29
13.23
14.14
15.74
16.01
18.03
19.54
19.72
20.36
20.93
22.62
25.20
27.63
±0.2 degrees 2 theta.

In some embodiments, the anion is L-lactate (i.e., the salt is a L-lactate salt).

In some embodiments, the L-lactate salt is characterized by peaks in an XRPD pattern at 7.0±0.2, 10.1±0.2, and 11.2±0.2° 2θ. In some embodiments, the L-lactate salt is further characterized by at least one XRPD peak selected from 15.9±0.2, 17.9±0.2, 20.8±0.2, 22.4±0.2, and 24.9±0.2° 2θ.

In some embodiments, the L-lactate salt is characterized by peaks in an XRPD pattern at 7.0±0.2, 10.1±0.2, 11.2±0.2, 15.9±0.2, 17.9±0.2, 20.8±0.2, 22.4±0.2, and 24.9±0.2° 2θ. In some embodiments, the L-lactate salt is further characterized by at least one XRPD peak selected from 10.9±0.2, 13.2±0.2, 13.9±0.2, 15.1±0.2, 15.6±0.2, 18.3±0.2, 19.0±0.2, 19.9±0.2, 21.2±0.2, 21.8±0.2, 22.9±0.2, 23.4±0.2, 23.8±0.2, 24.5±0.2, 25.8±0.2, 27.1±0.2, 27.3±0.2, 28.2±0.2, 29.5±0.2, 30.7±0.2, 31.4±0.2, 34.0±0.2, 35.7±0.2, 37.4±0.2, and 38.1±0.2° 2θ.

In some embodiments, the L-lactate salt is characterized by peaks in an XRPD

pattern at 7.0±0.2, 10.1±0.2, 10.9±0.2, 11.2±0.2, 13.2±0.2, 13.9±0.2, 15.1±0.2, 15.6±0.2, 15.9±0.2, 17.9±0.2, 18.3±0.2, 19.0±0.2, 19.9±0.2, 20.8±0.2, 21.2±0.2, 21.8±0.2, 22.4±0.2, 22.9±0.2, 23.4±0.2, 23.8±0.2, 24.5±0.2, 24.9±0.2, 25.8±0.2, 27.1±0.2, 27.3±0.2, 28.2±0.2, 29.5±0.2, 30.7±0.2, 31.4±0.2, 34.0±0.2, 35.7±0.2, 37.4±0.2, and 38.1±0.2° 2θ.

In some embodiments, the L-lactate salt is characterized by an XRPD pattern substantially similar to that shown in FIG. 40.

In some embodiments, the L-lactate salt exhibits a weight less of about 3% up to a temperature of about 150° C. as measured by TGA.

In some embodiments, the L-lactate salt exhibits a DSC thermogram comprising an endothermic peak at about 226±2.5° C.

In some embodiments, the anion is L-Lactate and the mitragynine salt exhibits an XRPD spectrum with copper radiation having 2 theta peaks at about:

Pos. [°2θ]

7.01
10.12
11.20
15.57
15.90
17.88
19.44
19.91
20.80
22.42
24.90
±0.2 degrees 2 theta.

In some embodiments, the anion is succinate (i.e., the salt is a succinate salt).

In some embodiments, the succinate salt is characterized by peaks in an XRPD pattern at 8.5±0.2, 17.6±0.2, and 19.3±0.2° 2θ. In some embodiments, the succinate salt is further characterized by at least one XRPD peak selected from 9.6±0.2, 14.4±0.2, 21.7±0.2, 23.1±0.2, 25.5±0.2, and 25.9±0.2° 2θ.

In some embodiments, the succinate salt is characterized by peaks in an XRPD pattern at 8.5±0.2, 9.6±0.2, 14.4±0.2, 17.6±0.2, 21.7±0.2, 23.1±0.2, 25.5±0.2, and 25.9±0.2° 2θ. In some embodiments, the succinate salt is further characterized by at least one XRPD peak selected from 6.2±0.2, 9.1±0.2, 10.1±0.2, 13.5±0.2, 15.7±0.2, 16.1±0.2, 17.0±0.2, 18.7±0.2, 21.2±0.2, 22.3±0.2, 23.7±0.2, 24.5±0.2, 25.0±0.2, 25.3±0.2, 27.0±0.2, 28.6±0.2, 29.1±0.2, 30.5±0.2, 31.6±0.2, 33.0±0.2, 34.3±0.2, 34.6±0.2, 37.0±0.2, and 39.1±0.2° 2θ.

In some embodiments, the succinate salt is characterized by peaks in an XRPD

pattern at 6.2±0.2, 8.5±0.2, 9.1±0.2, 9.6±0.2, 10.1±0.2, 13.5±0.2, 14.4±0.2, 15.7±0.2, 16.1±0.2, 17.0±0.2, 17.6±0.2, 18.7±0.2, 21.2±0.2, 21.7±0.2, 22.3±0.2, 23.1±0.2, 23.7±0.2, 24.5±0.2, 25.0±0.2, 25.3±0.2, 25.5±0.2, 25.9±0.2, 27.0±0.2, 28.6±0.2, 29.1±0.2, 30.5±0.2, 31.6±0.2, 33.0±0.2, 34.3±0.2, 34.6±0.2, 37.0±0.2, and 39.1±0.2° 2θ.

In some embodiments, the succinate salt is characterized by an XRPD pattern substantially similar to that shown in FIG. 42.

In some embodiments, the succinate salt exhibits a weight less of about 4% up to a temperature of about 150° C. as measured by TGA.

In some embodiments, the succinate salt exhibits a DSC thermogram comprising an endothermic peak at about 198±2.5° C. and about 202±2.5° C.

In some embodiments, the anion is succinate and the mitragynine salt exhibits an XRPD spectrum with copper radiation having 2 theta peaks at about:

Pos. [°2θ]

8.47
9.62
14.36
19.27
21.71
23.13
25.52
25.93
31.27
±0.2 degrees 2 theta.

In some embodiments, the anion is fumarate (i.e., the salt is a fumarate salt).

In some embodiments, the fumarate salt is characterized by peaks in an XRPD pattern at 8.3±0.2, 19.1±0.2, and 19.2±0.2° 2θ.

In some embodiments, the fumarate salt is further characterized by at least one XRPD peak selected from 14.3±0.2, 17.3±0.2, 18.6±0.2, 25.2±0.2, and 25.6±0.2° 2θ.

In some embodiments, the fumarate salt is characterized by peaks in an XRPD pattern at 8.3±0.2, 14.3±0.2, 17.3±0.2, 18.6±0.2, 19.1±0.2, and 19.2±0.2, 25.2±0.2, and 25.6±0.2° 2θ. In some embodiments, the fumarate salt is further characterized by at least one XRPD peak selected from 9.5±0.2, 15.1±0.2, 15.5±0.2, 16.0±0.2, 16.7±0.2, 19.9±0.2, 21.2±0.2, 21.4±0.2, 22.2±0.2, 22.9±0.2, 23.2±0.2, 23.6±0.2, 24.5±0.2, 26.8±0.2, 28.8±0.2, 31.0±0.2, and 34.1±0.2° 2θ.

In some embodiments, the fumarate salt is characterized by peaks in an XRPD pattern at 8.3±0.2, 9.5±0.2, 14.3±0.2, 15.1±0.2, 15.5±0.2, 16.0±0.2, 16.7±0.2, 17.3±0.2, 18.6±0.2, 19.1±0.2, 19.2±0.2, 19.9±0.2, 21.2±0.2, 21.4±0.2, 22.2±0.2, 22.9±0.2, 23.2±0.2, 23.6±0.2, 24.5±0.2, 25.3±0.2, 25.6±0.2, 26.8±0.2, 28.8±0.2, 31.0±0.2, and 34.1±0.2° 2θ.

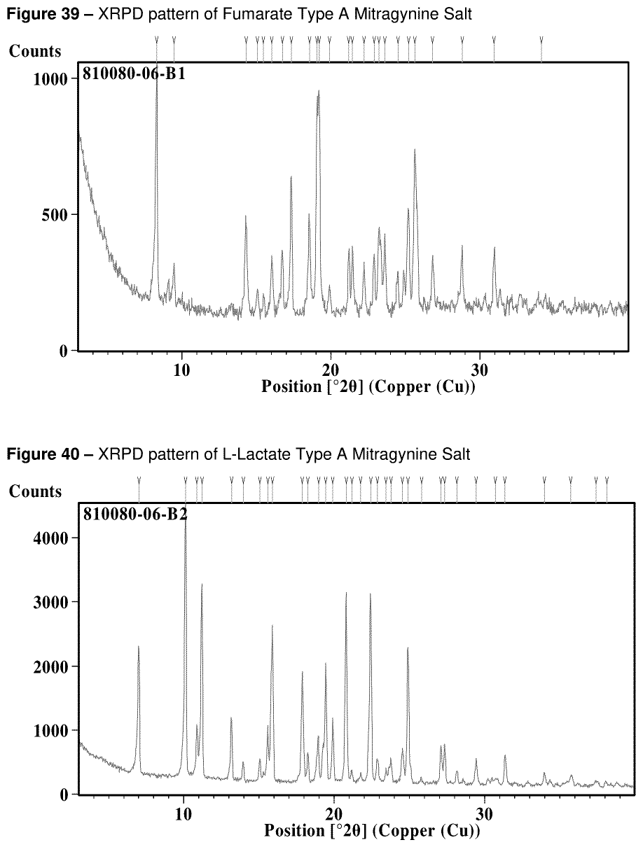

In some embodiments, the fumarate salt is characterized by an XRPD pattern substantially similar to that shown in FIG. 39.

In some embodiments, the fumarate salt exhibits a weight less of about 3% up to a temperature of about 150° C. as measured by TGA.

In some embodiments, the fumarate salt exhibits a DSC thermogram comprising an endothermic peak at about 226±2.5° C.

In some embodiments, the anion is fumarate and the mitragynine salt exhibits an XRPD spectrum with copper radiation having 2 theta peaks at about;

Pos. [°2θ]

8.30
14.29
16.03
16.73
17.33
18.55
19.06
19.22
21.19
21.44
22.89
23.61
25.20
25.62
28.80
±0.2 degrees 2 theta.

In some embodiments, the anion is mesylate (i.e., the salt is a mesylate salt).

In some embodiments, the mesylate salt is characterized by peaks in an XRPD pattern at 6.7±0.2, 16.7±0.2, and 17.4±0.2° 2θ. The salt of embodiment 129, wherein the mesylate salt is further characterized by at least one XRPD peak selected from 11.6±0.2, 18.6±0.2, 18.9±0.2, 20.1±0.2, and 26.0±0.2° 2θ.

In some embodiments, the mesylate salt is characterized by peaks in an XRPD pattern at 6.7±0.2, 11.6±0.2, 16.7±0.2, 17.4±0.2, 18.6±0.2, 18.9±0.2, 20.1±0.2, and 26.0±0.2° 2θ. In some embodiments, the mesylate salt is further characterized by at least one XRPD peak selected from 8.2±0.2, 10.0±0.2, 13.0±0.2, 13.4±0.2, 15.3±0.2, 16.4±0.2, 18.3±0.2, 19.8±0.2, 21.2±0.2, 21.4±0.2, 22.2±0.2, 22.7±0.2, 23.0±0.2, 23.7±0.2, 24.2±0.2, 24.4±0.2, 24.6±0.2, 25.1±0.2, 26.8±0.2, 27.1±0.2, 28.5±0.2, 30.1±0.2, 32.9±0.2, 33.7±0.2, and 37.1±0.2° 2θ.

In some embodiments, the mesylate salt is characterized by peaks in an XRPD

pattern at 6.7±0.2, 8.2±0.2, 10.0±0.2, 11.6±0.2, 13.0±0.2, 13.4±0.2, 15.3±0.2, 16.4±0.2, 16.7±0.2, 17.4±0.2, 18.3±0.2, 18.6±0.2, 18.9±0.2, 19.8±0.2, 20.1±0.2, 21.2±0.2, 21.4±0.2, 22.2±0.2, 22.7±0.2, 23.0±0.2, 23.7±0.2, 24.2±0.2, 24.4±0.2, 24.6±0.2, 25.1±0.2, 26.0±0.2, 26.8±0.2, 27.1±0.2, 28.5±0.2, 30.1±0.2, 32.9±0.2, 33.7±0.2, and 37.1±0.2° 2θ.

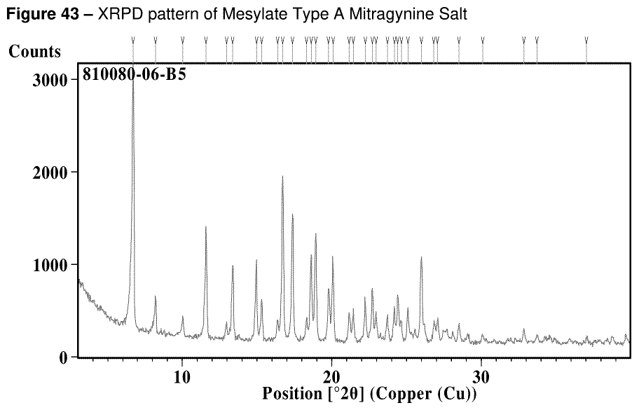

In some embodiments, the mesylate salt is characterized by an XRPD pattern substantially similar to that shown in FIG. 43.

In some embodiments, the mesylate salt exhibits a weight less of about 2% up to a temperature of about 150° C. as measured by TGA.

In some embodiments, the mesylate salt exhibits a DSC thermogram comprising an endothermic peak at about 275±2.5° C.

In some embodiments, the anion is mesylate and the mitragynine salt exhibits an XRPD spectrum with copper radiation having 2 theta peaks at about:

Pos. [°2θ]

6.71
11.59
13.38
14.94
16.71
17.37
18.62
18.93
20.07
25.99
±0.2 degrees 2 theta.

In one aspect the present disclosure provides a crystalline glycolate salt of mitragynine.

In one aspect the present disclosure provides a glycolate Type A salt of mitragynine exhibiting an XRPD spectrum with copper radiation having 2 theta peaks at about:

Pos. [°2θ]

7.09
10.16
11.29
13.23
14.14
15.74
16.01
18.03
19.54
19.72
20.36
20.93
22.62
25.20
27.63
±0.2 degrees 2 theta.

In some embodiments, a glycolate Type B salt of mitragynine is provided.

In some embodiments, a glycolate Type C salt of mitragynine is provided.

In some embodiments, a glycolate Type D salt of mitragynine is provided.

In some embodiments, a glycolate Type E salt of mitragynine is provided.

In some embodiments, a glycolate Type F salt of mitragynine is provided.

### Methods of Making the Salts of the Present Disclosure

In one aspect there is provided a process for producing a glycolate salt of 3-deuteromitragynine, the process including the step of crystallizing a glycolate salt of 3-deuteromitragynine from a solution of isopropyl alcohol.

In some embodiments, the solution of isopropyl alcohol includes water.

In one aspect there is provided a process for producing a glycolate salt of mitragynine, the process including the step of crystallizing a glycolate salt of mitragynine from a solution of isopropyl alcohol.

In some embodiments, the solution of isopropyl alcohol includes water.

In some embodiments, the glycolate salt of mitragynine is derived from a crude alkaloid extract of Mitragyna speciosa.

In one aspect there is provided a process of purifying 3-deuteromitragynine or mitragynine, the purification process including a step of crystallizing any one of the 3-deuteromitragynine or mitragynine salts as defined above.

In some embodiments, the purified mitragynine is derived from a crude alkaloid extract of Mitragyna speciosa.

In some embodiments, the purification of 3-deuteromitragynine or mitragynine is comprises crystallizing the glycolate, L-lactate, succinate, fumarate, or mesylate salt.

In some embodiments, the purification of 3-deuteromitragynine or mitragynine comprises crystallizing the glycolate salt.

In some embodiments, the purification of 3-deuteromitragynine or mitragynine comprises crystallizing the L-lactate salt.

In some embodiments, the purification of 3-deuteromitragynine or mitragynine comprises crystallizing the succinate salt.

In some embodiments, the purification of 3-deuteromitragynine or mitragynine comprises crystallizing the fumarate salt.

In some embodiments, the purification of 3-deuteromitragynine or mitragynine comprises crystallizing the mesylate salt.

In some embodiments, the purified 3-deuteromitragynine or mitragynine salt is at least 90% free of other compounds or impurities.

In some embodiments, the purified 3-deuteromitragynine or mitragynine salt is at least 95% free of other compounds or impurities.

In some embodiments, the purified 3-deuteromitragynine or mitragynine salt is at least 98% free of other compounds or impurities.

In some embodiments, the purified 3-deuteromitragynine or mitragynine salt is at least 99% free of other compounds or impurities.

In some embodiments, the purified 3-deuteromitragynine salt has less than about 3% of the impurity 3-deuterocorynantheidine (3-DCR).

In some embodiments, the purified 3-deuteromitragynine salt has less than about 2% of the impurity 3-DCR.

In some embodiments, the purified 3-deuteromitragynine salt has less than about 1% of the impurity 3-DCR.

In some embodiments, the purified 3-deuteromitragynine salt has less than about 0.5% of the impurity 3-DCR.

In some embodiments, the purified mitragynine salt has less than about 3% of the impurity corynantheidine (CR).

In some embodiments, the purified mitragynine salt has less than about 2% of the impurity CR.

In some embodiments, the purified mitragynine salt has less than about 1% of the impurity CR.

In some embodiments, the purified mitragynine salt has less than about 0.5% of the impurity CR.

### Pharmaceutical Compositions

The present disclosure further provides a pharmaceutical composition comprising an amount of one or more salts of 3-deuteromitragynine having the structure:

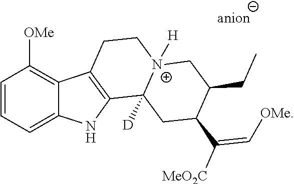 [SMILES: [2H][C@@]12C[C@H](/C(=C\OC)C(=O)OC)[C@H](CC)C[NH+]1CCc1c2[nH]c2cccc(OC)c12]

In some embodiments, the anion is glycolate, L-lactate, succinate, fumarate, or mesylate.

In some embodiments, the anion is glycolate.

In some embodiments, the anion is L-lactate.

In some embodiments, the anion is succinate.

In some embodiments, the anion is fumarate.

In some embodiments, the anion is mesylate.

In some embodiments, the composition further includes a pharmaceutically acceptable carrier.

The present disclosure further provides a pharmaceutical composition comprising an amount of one or more salts of mitragynine having the structure:

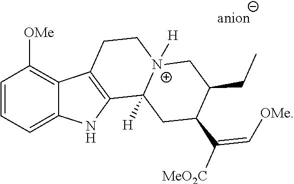 [SMILES: CC[C@@H]1C[NH+]2CCc3c([nH]c4cccc(OC)c34)[C@@H]2C[C@@H]1/C(=C\OC)C(=O)OC]

In some embodiments, the anion is glycolate, L-lactate, succinate, fumarate, or mesylate.

In some embodiments, the anion is glycolate.

In some embodiments, the anion is L-lactate.

In some embodiments, the anion is succinate.

In some embodiments, the anion is fumarate.

In some embodiments, the anion is mesylate.

In some embodiments, the composition further includes a pharmaceutically acceptable carrier.

As used herein, a “pharmaceutically acceptable carrier” is a pharmaceutically acceptable solvent, suspending agent or vehicle, for delivering the instant salt(s) to the animal or human. The carrier may be liquid or solid and is selected with the planned manner of administration in mind. Liposomes are also a pharmaceutically acceptable carrier, as are capsules, tablets, coatings, and various syringes.

A dosage unit of the salt(s) used in the method of the present disclosure may comprise a single salt or mixtures thereof. The salt(s) can be administered in oral dosage forms as tablets, capsules, pills, powders, granules, elixirs, tinctures, suspensions, syrups, and emulsions. The salt(s) may also be administered in intravenous (bolus or infusion), intraperitoneal, subcutaneous, or intramuscular form, or introduced directly, e.g. by injection, topical application, or other methods, into or onto a site of disease, all using dosage forms well known to those of ordinary skill in the pharmaceutical arts.

The salts used in the present disclosure can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. Extended-release formulations are specifically encompassed. The unit will be in a form suitable for oral, rectal, topical, intravenous or direct injection or parenteral administration. The salt(s) can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present disclosure are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The salt(s) used in the method of the present disclosure may also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines.

Gelatin capsules may contain the salt(s) and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The salt(s) used in the present disclosure may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

### Methods of Treatment

In one aspect there is provided a method of treating a subject afflicted with acute pain or chronic pain, a depressive disorder, a mood disorder, an anxiety disorder, borderline personality disorder, a substance use disorder, opioid use disorder, opioid withdrawal symptoms, alcohol use disorder, or alcohol withdrawal disorder, comprising administering an effective amount of a salt or a composition as defined above to the subject so as to thereby treat the subject afflicted with acute pain or chronic pain, the depressive disorder, mood disorder, anxiety disorder, borderline personality disorder, a substance use disorder, opioid use disorder, opioid withdrawal symptoms, alcohol use disorder, or alcohol withdrawal disorder.

In some embodiments, the present disclosure provides methods of treating a subject afflicted with acute pain, chronic pain, a depressive disorder, a mood disorder, an anxiety disorder, borderline personality disorder, a substance use disorder, opioid use disorder, opioid withdrawal symptoms, alcohol use disorder, or alcohol withdrawal disorder, comprising administering an effective amount of a salt of any of the salts or pharmaceutical compositions described herein to the subject.

The method of embodiment 139, the subject is afflicted with an opioid use disorder.

The method of embodiment 140, wherein the subject is afflicted with opioid withdrawal.

Administration of one or more salt(s) and/or one or more compositions (e.g., pharmaceutical compositions) disclosed herein may be used for preventing, slowing, halting, or reversing the progression of acute pain or chronic pain, a depressive disorder, a mood disorder, an anxiety disorder, borderline personality disorder, a substance use disorder, opioid use disorder, opioid withdrawal symptoms, alcohol use disorder, or alcohol withdrawal disorder as set out herein. Administration may also improve one or more symptoms of acute pain or chronic pain, a depressive disorder, a mood disorder, an anxiety disorder, borderline personality disorder, a substance use disorder, opioid use disorder, opioid withdrawal symptoms, alcohol use disorder, or alcohol withdrawal disorder.

The salt(s) used in the method of the present disclosure may be administered in various forms, including those detailed herein. The treatment with the salt(s) may be a component of a combination therapy or an adjunct therapy, i.e. the subject or patient in need of the drug is treated or given another drug for the disease in conjunction with one or more of the instant salt(s). This combination therapy can be sequential therapy where the patient is treated first with one drug and then the other or the two drugs are given simultaneously. These can be administered independently by the same route or by two or more different routes of administration depending on the dosage forms employed.

The dosage of the salt(s) administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

In some embodiments, a disclosed salt may be administered at a dosage unit of about 0.1 mg to about 1000 mg, or about 1 mg to about 400 mg, or about 5 mg to about 300 mg, about 10 mg to about 200 mg, about 100 mg to about 200 mg, or at least 400 mg, at least 300 mg, at least 200 mg, at least 150 mg, at least 120 mg, at least 100 mg, at least 50 mg, at least 40 mg, at least 30 mg, at least 20 mg, at least 10 mg, at least 9 mg, at least 9.5 mg, at least 8 mg, at least 7.5 mg, at least 7 mg, at least 6.5 mg, at least 6 mg, at least 5.5 mg, at least 5 mg, at least 4.5 mg, at least 4 mg, at least 3.5 mg, at least 3 mg, at least 2.5 mg, at least 2 mg, or at least 1 mg.

In some embodiments, about 5 mg to about 100 mg, including about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg or about 100 mg, including all values and ranges therebetween, of a salt of deuterated mitragynine as described herein is administered to a patient in need thereof. In some embodiments, about 10 mg to about 90 mg of a salt of deuterated mitragynine as described herein is administered to a patient in need thereof.

Each embodiment disclosed herein is contemplated as being applicable to each of the other disclosed embodiments. Thus, all combinations of the various elements described herein are within the scope of this disclosure.

This invention will be better understood by reference to the Experimental Details which follow, but those skilled in the art will readily appreciate that the specific experiments detailed are only illustrative and that the invention is described in the claims which follow thereafter.

### EXAMPLES

### General Experimental Methods

The general analytical methods used throughout the following examples are described below.

### X-Ray Powder Diffraction (XRPD)

For XRPD analysis, Empyrean and X′pert 3 X-ray powder diffractometers were used. Samples were spread on the middle of a zero-background Si holder. The XRPD parameters used are described in Table 1.

TABLE 1
Parameters for XRPD testing

| Model | Empyrean | X'Pert3 |
|---|---|---|
| X-Ray wavelength | Cu, kα, Kα1 (Å): 1.540598, Kα2 (Å): 1.544426 | |
| | Kα2/Kα1 intensity ratio: 0.50 | |
| X-Ray tube setting | 45 kV, 40 mA | 45 kV, 40 mA |
| Divergence slit | Automatic | ⅛° |
| Scan mode | Continuous | Continuous |
| Scan range (°2TH) | 3°-40° | 3°-40° |
| Scan step time (s) | 33.020 | 46.667 |
| Step size (°2TH) | 0.0167 | 0.0263 |
| Test time | 10 min 10 s | 5 min 4 s |

Thermogravimetric Analysis (TGA) and Differential Scanning calorimetry (DSC)

TGA data were collected using a TA Q5000 and Discovery TGA 5500 TGA from TA Instruments. DSC and mDSC were performed using a TA Q2000 DSC and Discovery DSC 2500 from TA Instruments. The detailed parameters used are described in Table 2 and Table 3.

TABLE 2
Parameters for TGA and DSC testing

| Parameters | TGA | DSC |
|---|---|---|
| Method | Ramp | Ramp |
| Sample pan | Aluminum, open | Aluminum, crimped |
| Temperature | RT to 350° C. | 25° C. to target |
| | | temperature |
| Heating rate | 10° C./min | 10° C./min |
| Purge gas | N₂ | N₂ |

TABLE 3
Parameters for mDSC testing

| Parameters | mDSC | |
|---|---|---|
| Sample pan | Aluminum, crimped | |
| Temperature | 0-250° | C. |
| Heating rate | 3° | C./min |
| Purge gas | N₂ | |
| Amplitude | ±1° | C. |
| Modulation period | 60 | s |

### Dynamic Vapor Sorption (DVS)

DVS was measured via an SMS (Surface Measurement Systems) DVS Intrinsic. The relative humidity at 25° C. was calibrated against the deliquescence point of LiCl, Mg(NOs)₂, and KCl. Parameters for DVS testing are described in Table 4.

TABLE 4
Parameters for DVS testing.

| Parameters | DVS | |
|---|---|---|
| Temperature | 25° | C. |
| Sample size | 10~20 | mg |
| Gas and flow rate | N₂, 200 | mL/min |
| dm/dt | 0.002%/min | |
| Min. Dm/dtstabilityduration | 10 | min |
| Max. equilibrium time | 180 | min |

| | |
|---|---|
| RH range | 0% RH~95% RH~0% RH |
| RH step size | 10% RH from 0% RH to 90% RH & |
| | 90% RH to 0% RH |
| | 5% RH from 90% RH to 95% RH & |
| | 95% RH to 90% RH |

### High Pressure Liquid Chromatography (HPLC)

An Agilent 1260 HPLC with DAD/VWD detector and Agilent 1100 with DAD detector were utilized. Detailed chromatographic conditions for purity and stoichiometric ratio analysis are described in Table 5-8.

TABLE 5
Chromatographic conditions for purity analysis, Method 1.

| | Agilent 1260 HPLC with DAD/VWD detector & | |
|---|---|---|
| HPLC | Agilent 1100 HPLC with DAD detector | |
| Column | Supelco Ascentis Express 2.7 μm 4.6 × 100 mm | |
| Mobile phase | A: H₂O/MeOH (80:20, v/v) + 0.1% H₃PO₄ | |
| | B: MeOH + 0.1% H₃PO₄ | |
| | Time (min) | % B |
| Gradient table | 0.0 | 0 |
| | 20.0 | 50 |
| | 25.0 | 50 |
| | 26.0 | 70 |
| | 27.0 | 0 |
| | 30.0 | 0 |

| | |
|---|---|
| Run time | 30.0 |
| Post time | 0.0 |
| Flow rate | 1.0 mL/min |
| Injection volume | 5 μL |
| Detector wavelength | 220 nm |
| Column temperature | 50° C. |
| Sampler temperature | RT |
| Diluent | MeOH |

TABLE 6
Chromatographic conditions for purity analysis, Method 2.

| HPLC | Agilent 1260 HPLC with DAD/VWD detector | |
|---|---|---|
| Column | ZORBAX SB-Phenyl C18, 4.6 × 150 mm, 3.5 μm | |
| Mobile phase | A: H₂O/MeOH (80:20, v/v) + 0.1% H₃PO₄ | |
| | B: MeOH + 0.1% H₃PO₄ | |
| | Time (min) | % B |
| Gradient table | 0.0 | 0 |
| | 20.0 | 50 |
| | 25.0 | 50 |
| | 26.0 | 70 |
| | 27.0 | 0 |
| | 30.0 | 0 |

| | |
|---|---|
| Run time | 30.0 |
| Post time | 0.0 |
| Flow rate | 1.0 mL/min |
| Injection volume | 5 μL |
| Detector wavelength | 220 nm |
| Column temperature | 50° C. |
| Sampler temperature | RT |
| Diluent | MeOH |

TABLE 7
Chromatographic conditions for stoichiometric
ratio and solubility analysis, Method 1.

| | Agilent 1260 HPLC with DAD/VWD detector & | |
|---|---|---|
| HPLC | Agilent 1100 HPLC with DAD detector | |
| Column | Supelco Ascentis Express 2.7 μm 4.6 × 100 mm | |
| Mobile phase | A: H₂O/MeOH (80:20, v/v) + 0.1% H₃PO₄ | |
| | B: MeOH + 0.1% H₃PO₄ | |
| | Time (min) | % B |
| Gradient table | 0.00 | 0 |
| | 8.00 | 60 |
| | 10.00 | 60 |
| | 10.01 | 0 |
| | 12.00 | 0 |

| | |
|---|---|
| Run time | 12.0 min |
| Post time | 0.0 min |
| Flow rate | 1.0 mL/min |
| Injection volume | 5 μL |
| Detector wavelength | 220 nm |
| Column temperature | 50° C. |
| Sampler temperature | RT |
| Diluent | MeOH |

TABLE 8
Chromatographic conditions for stoichiometric
ratio and solubility analysis, Method 2.

| HPLC | Agilent 1260 HPLC with DAD/VWD detector | |
|---|---|---|
| Column | ZORBAX SB-Phenyl C18, 4.6 × 150 mm, 3.5 μm | |
| Mobile phase | A: H₂O/MeOH (80:20, v/v) + 0.1% H₃PO₄ | |
| | B: MeOH + 0.1% H₃PO₄ | |
| | Time (min) | % B |
| Gradient table | 0.00 | 0 |
| | 8.00 | 60 |
| | 10.00 | 60 |
| | 10.01 | 0 |
| | 12.00 | 0 |

| | |
|---|---|
| Run time | 12.0 min |
| Post time | 0.0 min |
| Flow rate | 1.0 mL/min |
| Injection volume | 5 μL |
| Detector wavelength | 220 nm |
| Column temperature | 50° C. |
| Sampler temperature | RT |
| Diluent | MeOH |

### Ion Chromatography (IC)

A ThermoFisher ICS-1100 was utilized for ion chromatography (IC) and detailed conditions are described in Table 9.

TABLE 9
Ion chromatography conditions and parameters.

| IC | ThermoFisher ICS-1100 | |
|---|---|---|
| Column | IonPac AS18 Analytical Column (4 × 250 mm) | |
| Mobile phase | 25 | mM NaOH |
| Injection volume | 25 | μL |
| Flow rate | 1.0 | mL/min |
| Cell temperature | 35° | C. |
| Column temperature | 35° | C. |
| Current | 80 | mA |

| | |
|---|---|
| Run time | 7.0 min for Cl⁻, 16.0 min |
| | for C₂O₄²⁻, 40.0 min |
| | for PO₄³⁻ |

### Polarized Light Microscopy (PLM)

PLM images were captured with a ZEISS Scope.A1 microscope.

### Karl Fischer (KF) Titration

The instrument (Metrohm 870 KF Titrinoplus) was calibrated using purified water and the titration reagent was Hydranal® R-Composite 5 provided by Sigma-Aldrich. HPLC grade methanol was used to dissolve samples.

### ¹H Solution Nuclear Magnetic Resonance (NMR)

¹H solution NMR was collected on a Bruker 400 MHZ NMR spectrometer using deuterated DMSO as solvent.

### Example 1. Preparation and Characterization of 3-Deuteromitragynine (3-DM) Free Base for Salt Screening

3-Deuteromitragynine (3-DM) free base for use in salt and polymorph screening experiments was prepared as previously described in WO2020160280—entitled “Deuterated mitragynine analogs as safer opioid modulators in the mitragynine class, which reference is incorporated herein by reference.” Reagents and solvents were obtained from commercial sources and were used without further purification unless otherwise stated. Reactions were monitored by TLC using solvent mixtures appropriate to each reaction. All column chromatography was performed on silica gel (40-63 μm). Preparative TLC was conducted on glass plates coated with a 1 mm silica layer. Nuclear magnetic resonance spectra were recorded on Bruker 400 or 500 MHz instruments, as indicated. Chemical shifts are reported as δ values in ppm referenced to CDCl₃ (¹H NMR=7.26 and ¹³C NMR=77.16) or methanol-d₄ (¹H NMR=3.31 and ¹³C NMR=49.00). Multiplicity is indicated as follows: s (singlet); d (doublet); t (triplet); dd (doublet of doublets); td (triplet of doublets); dt (doublet of triplets); ddd (doublet of doublet of doublets); m (multiplet); br (broad). All carbon peaks are rounded to one decimal place unless such rounding would cause two close peaks to become identical; in these cases, two decimal places are retained. Low-resolution mass spectra were recorded on an Advion quadrupole instrument (ionization mode: APCI+). Percent deuteration was determined by mass spectrometry on a high-resolution quadrupole-time-of-flight instrument (ionization mode: ESI+) by quantitative comparison of the isotope pattern of deuterated compounds to controls having natural isotopic abundance. The following Scheme 1 also shows the process involved in preparing 3-DM.

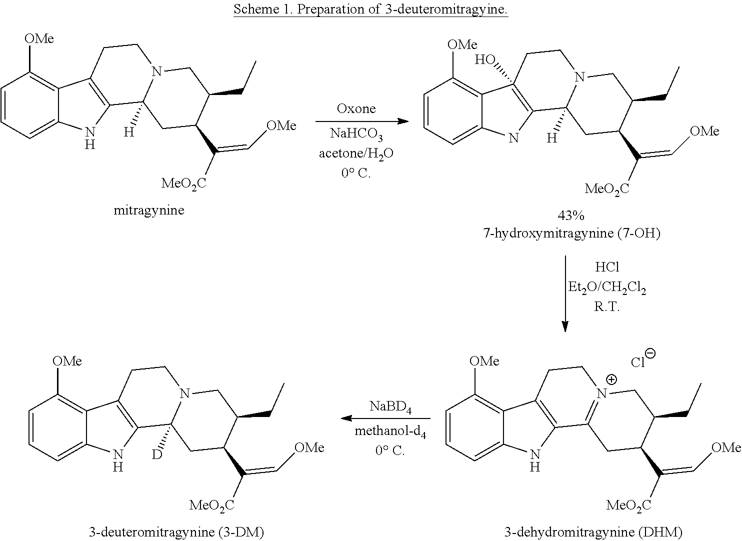

Mitragynine. Mitragynine free base was obtained by extraction from powdered Mitragyna speciosa leaves as previously described (Kruegel et al. 2016). Spectral and physical properties were in agreement with those previously reported (Kruegel et al. 2016).

7-Hydroxymitragynine (7-OH) Procedure 1). Mitragynine (1.99 g, 5.00 mmol) was dissolved in acetone (100 mL), saturated aqueous NaHCO₃ (10 mL) was added, and the mixture was cooled to 0° C. A solution of Oxone monopersulfate (2KHSO₅·KHSO₄·K₂SO₄; 2.31 g, 3.75 mmol) in water (10 mL) was then added dropwise over 35 minutes and the mixture left to stir at 0° C. After 45 minutes, additional Oxone monopersulfate (769 mg, 1.25 mmol) in water (3.3 mL) was added over ˜2 minutes and stirring was continued at 0° C. for an additional 15 minutes. At this time, the reaction was diluted with water (150 mL) and extracted with EtOAc (3×50 mL). The combined organics were washed with brine (50 mL), dried over Na₂SO₄, and concentrated in vacuo to give the crude product as a tan foam (1.42 g). This material was purified by column chromatography (6:4 hexanes:EtOAc+2% Et₃N) to provide pure 7-hydroxymitragynine as an amorphous, pale-yellow solid (882 mg. 43%). Spectral and physical properties were in agreement with those previously reported (Kruegel et al. 2016).

7-Hydroxymitragynine (7-OH) (Procedure 2—Larger Scale). Mitragynine (9.96 g, 25.00 mmol) was dissolved in acetone (750 mL), saturated aqueous NaHCO₃ (500 mL) was added, and the mixture was cooled to 0° C. A solution of Oxone monopersulfate (2KHSO₅·KHSO₄·K₂SO₄; 15.39 g, 25.00 mmol) in water (250 mL) was then pre-cooled to 0° C. and added dropwise over 30 minutes (mixture was hard to stir at first but became less viscous over the course of the addition). TLC at the end of the Oxone addition showed no starting material so the reaction was worked up (at 15 minutes after the end of the addition). EtOAc (500 mL) and water (500 mL) were added to the reaction mixture while it was still stirring at 0° C. and the resulting mixture was then poured into a separatory funnel containing additional water (1,000 mL). The organic layer was separated and the aqueous phase extracted with additional EtOAc (2×500 mL). The combined organics were washed with brine (300 mL), dried over Na₂SO₄, and concentrated in vacuo to give the crude product as a yellow ochre foam (7.35 g). This material was purified by silica column chromatography (320 g silica; 600 mL column volume; 60 mL fractions; step gradient: 20%→30%→35%→40%→45%→50%→55% EtOAc in hexanes+2% Et₃N, 1 column volume per step) to provide the following fractions: fractions 49-51=very pale-yellow amorphous solid, 7-hydroxymitragynine+˜2% 7-hydroxycorynantheidine, 1.09 g (11%); fractions 52-64=pale-yellow amorphous solid, 7-hydroxymitragynine, 2.99 g (29%). Spectral properties were in agreement with those previously reported (Kruegel et al. 2016).

3-Dehydromitragynine hydrochloride (DHM) (Procedure 1). To a solution of 7-hydroxymitragynine (746 mg, 1.80 mmol) in anhydrous CH₂Cl₂ (27 mL) under argon was added 2.0 M HC1 in Et₂0 (9.0 mL) and the resulting mixture was stirred at room temperature for 45 minutes (all solids dissolved to give a transparent yellow solution after 2-3 minutes). The reaction mixture was then concentrated directly in vacuo to give pure 3-dehydromitragynine hydrochloride as a yellow solid (797 mg, quantitative). ¹H NMR (500 MHz, CDCl₃) δ 13.56 (br s, 1H), 7.49 (s, 1H), 7.42 (d, J=8.4 Hz, 1H), 7.25 (t, J=8.0 Hz 1H), 6.38 (d, J=7.7 Hz, 1H), 4.03-3.81 (m, 3H), 3.89 (s, 3H), 3.80-3.65 (m, 1H), 3.76 (s, 3H), 3.65-3.53 (m, 2H), 3.61 (s, 3H), 3.53-3.36 (m, 2H), 3.29 (t, J=12.6 Hz, 1H), 2.10 (br s, 1H), 1.55-1.43 (m, 1H), 1.22-1.10 (m, 1H), 0.98 (t, J=7.4 Hz, 3H).

3-Dehydromitragynine hydrochloride (DHM) (Procedure 2—Larger Scale). To a solution of 7-hydroxymitragynine (14.09 g, 34.00 mmol) in anhydrous CH₂Cl₂ (510 mL) under argon was added 2.0 M HCl in Et₂0 (170 mL) (yellow suspension forms and slight warming occurs on HCl addition) and the resulting mixture was stirred at room temperature for 40 minutes (all solids dissolved to give a transparent yellow-orange solution after 2-3 minutes). The reaction mixture was then concentrated directly in vacuo to give pure 3-dehydromitragynine hydrochloride as a yellow solid (15.87 g, quantitative). The NMR spectra of this material were identical to those of material obtained via Procedure 1 above.

3-Deuteromitragynine (3-DM) (Procedure 1). To a solution of 3-dehydromitragynine hydrochloride (606 mg, 1.40 mmol) in methanol-d₄ (28 mL) at 0° C. was added NaBD₄ (293 mg. 7.00 mmol) and the yellow solution was stirred at 0° C. for 20 minutes. The reaction was then diluted with water (100 mL) and extracted with CH₂Cl₂ (3×50 mL). The combined organics were washed with water (2×50 mL), dried over Na₂SO₄, and concentrated in vacuo to give the crude product as a very pale-yellow foam (0.52 g). This material was purified by column chromatography (8:2 hexanes:EtOAc+2% Et₃N, 4 column volumes→7:3 hexanes:EtOAc+2% Et₃N, 3 column volumes) to provide pure 3-deuteromitragynine as an amorphous, off-white solid (480 mg, 86%). ¹H NMR (500 MHz, CDCl₃) d 7.70 (br s, 1H), 7.43 (s, 1H), 6.99 (t, J=7.9 Hz, 1H), 6.90 (d, J=7.7 Hz, 1H), 6.46 (d, J=7.7 Hz, 1H), 3.88 (s, 3H), 3.73 (s, 3H), 3.71 (s, 3H), 3.12 (ddd, J=15.8, 11.6, 5.9 Hz, 1H), 3.07-2.89 (m, 4H), 2.58-2.42 (m, 3H), 1.85-1.73 (m, 2H), 1.66-1.58 (m, 1H), 1.25-1.15 (m, 1H), 0.87 (t, J=7.4 Hz, 3H);

¹³C NMR (126 MHz, CDCl₃) δ 169.4, 160.7, 154.6, 137.4, 133.8, 121.9, 117.7, 111.6, 107.9, 104.4, 99.8, 61.6, 60.9 (t, JCD=19.5 Hz), 57.9, 55.4, 53.9, 51.5, 40.8, 40.1, 29.9, 24.1, 19.2, 13.0; HR-MS calcd. for C₂₃H₃₀DN₂O₄ [M+H]⁺ 400.2341, found 400.2332; Deuterium Enrichment=° 97.5-97.7 atom % D (by HR-MS).

3-Deuteromitragynine (3-DM) (Procedure 2). To a solution of 3-dehydromitragynine hydrochloride (54.1 mg, 0.125 mmol) in MeOH (2.5 mL) at 0° C. was added NaBD4 (26.2 mg, 0.625 mmol) and the yellow solution was allowed to warm to room temperature and stirred for 25 minutes. The reaction was then diluted with water (10 mL) and extracted with CH₂Cl₂ (3×5 mL). The combined organics were washed with water (2×5 mL), dried over Na₂SC>4, and concentrated in vacuo to give the crude product as a foamy yellow glass (47.2 mg). This material was purified by column chromatography (7:3 hexanes:EtOAc+2% Et₃N) to provide pure 3-deuteromitragynine as an amorphous, yellow solid (39.4 mg, 79%). The NMR spectra of this material were identical to those of material obtained via Procedure 1 above, with the exception of visible residual peaks for undeuterated mitragynine in both the proton and carbon spectra. Deuterium Enrichment=93.5-93.8 atom % D (by HR-MS).

3-Deuteromitragynine (3-DM) (Procedure 3). To a solution of 3-dehydromitragynine hydrochloride (14.72 g, 34.00 mmol=15.85 g of crude containing CH₂Cl₂ from last step) in methanol-OD (CH₃OD; 170 mL) at 0° C. was added NaBD4 (2.85 mg, 68.00 mmol) and the yellow solution (clouds immediately after NaBD4 addition) was stirred at 0° C. for 20 minutes (effervescence stops after 10 minutes). The reaction was then diluted with water (500 mL) and extracted with CH₂Cl₂ (3×250 mL). The combined organics were washed with water (2×250 mL), dried over Na₂SO₄, and concentrated in vacuo to give the crude product as a pale-yellow foam (14.28 g). This material was purified by silica column chromatography (320 g silica; 600 mL column volume; 60 mL fractions; step gradient: 10% (2 column volumes)→20% (2 column volumes)→30% (4 column volumes) EtOAc in hexanes+2% Et₃N, first 2 column volumes discarded) to provide the following fractions: fractions 19-45=cream-colored amorphous solid, 3-deuteromitragynine, 11.86 g (87%); fractions 17-18+46-55=pale-yellow amorphous solid, impure 3-deuteromitragynine, 0.66 g (˜5%). The NMR spectra of this material were identical to those of material obtained via Procedures 1 and 2 above. Deuterium Enrichment=98.2-98.4 atom % D (by HR-MS).

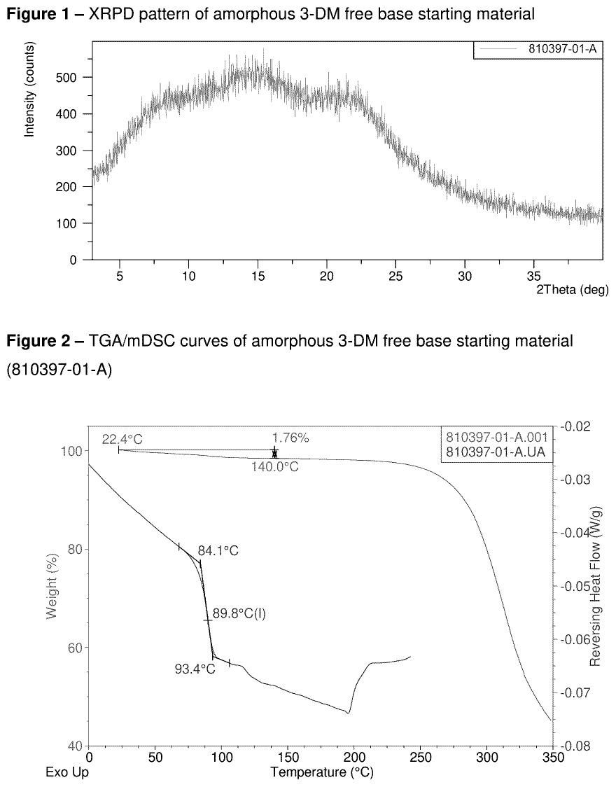

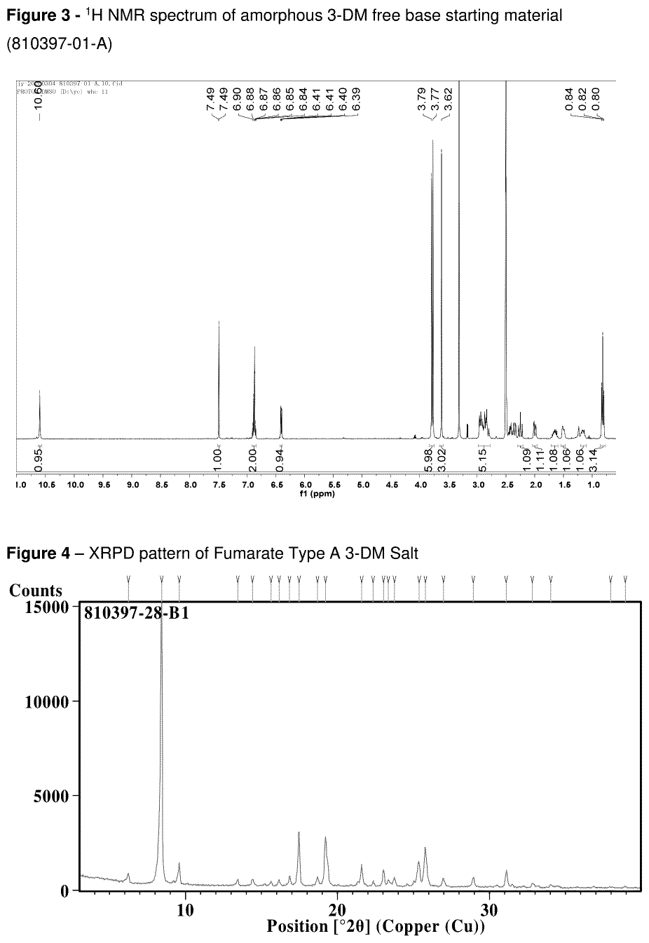

3-DM free base prepared by the above procedures for use in the salt and polymorph screening described below was characterized by XRPD, TGA, and mDSC and ¹H NMR. As the XRPD pattern in FIG. 1 shows, the starting material was amorphous. TGA/mDSC curves of starting material are shown in FIG. 2. The TGA curve showed a weight loss of 1.8% up to 140° C. The mDSC curve showed a glass transition temperature (Tg) at 89.8° C. (middle temperature). ¹H NMR data in FIG. 3 was collected using DMSO-d₆ as solvent.

The approximate solubility of the free base starting material was determined in 27 solvent systems at RT. Approximate 2 mg of material was added into a 3-mL glass vial. The solvents in Table 10 were then added stepwise into the vials until the solids were dissolved visually or a total volume of 1 mL was reached. The solubility results summarized in Table 10 were used to guide the solvent selection in salt screening design.

TABLE 10
Approximate solubility of amorphous 3-DM free
base starting material (810397-01-A) at RT.

| | Solubility | | Solubility |
|---|---|---|---|
| Solvent | (mg/mL) | Solvent | (mg/mL) |
| MeOH | S > 46.0 | MTBE | S > 46.0 |
| EtOH | S > 38.0 | THF | S > 40.0 |
| IPA | S > 36.0 | 2-MeTHF | S > 48.0 |
| Acetone | S > 38.0 | 1,4-dioxane | S > 42.0 |
| MIBK | S > 40.0 | ACN | S > 46.0 |
| EtOAc | S > 40.0 | DCM | S > 42.0 |
| IPAc | S > 42.0 | CHCl₃ | S > 46.0 |
| n-Heptane | S < 2.3 | IPA/H₂O (4:1) | 7.0 < S < 21.0 |
| Toluene | S > 36.0 | Acetone/n-Heptane | S > 42.0 |
| | | (4:1) | |
| DMSO | S > 34.0 | Acetone/n-Heptane | S > 38.0 |
| | | (1:1) | |
| DMAc | S > 46.0 | Acetone/n-Heptane | S > 46.0 |
| | | (1:4) | |
| NMP | S > 44.0 | Acetone/n-Heptane | 7.7 < S < 23.0 |
| | | (1:9) | |
| H₂O | S < 2.3 | Acetone/n-Heptane | 4.0 < S < 6.7 |
| | | (1:15) | |
| — | — | IPA/H₂O (9:1) | 23.0 < S < 46.0 |

The purity and 3-deuterocorynantheidine (3-DCR) content of the 3-DM free base starting material were also determined by HPLC, with the results summarized in Table 11.

TABLE 11
Impurities summary of amorphous 3-DM free
base starting material (810397-01-A).

| #Peak | RRT | Area (%) |
|---|---|---|
| 1 | 0.70 | 0.27 |
| 2 | 0.71 | 0.43 |
| 3 | 0.83 | 0.12 |
| 4 | 0.90 (3-DCR) | 2.68 |
| 5 | 0.94 | 0.21 |
| 6 | 0.95 | 0.08 |
| 7 | 1.00 (3-DM) | 96.21 |

### Example 2. Salt Screening of 3-Deuteromitragynine

According to the approximate solubility of the free base starting material at room temperature (RT, 24±2° C.) and simulated pKa value (7.33, basic), a total of 100 salt screening experiments were performed using 25 acids in four solvent systems via solvent-assisted reaction crystallization. For each experiment, about 20 mg of starting material and equimolar acid were mixed into each HPLC vial. 0.5 mL of corresponding solvent was then added to form a suspension, which was magnetically stirred (˜1,000 rpm) at RT for about three days. Solids were isolated and dried at 50° C. under vacuum for 4 hrs for XRPD analysis. As summarized in Table 12, a total of 26 crystalline salt hits and one crystalline form of free base were obtained during screening. Purity of all crystalline samples was tested, with results summarized in Table 13. All salt hits were characterized by XRPD, TGA, and DSC. The stoichiometric ratio was determined by ¹H NMR or HPLC/IC. Characterization results of salt hits and free base form are listed in Table 14.

TABLE 12
Summary of salt screening results for 3-DM.

| | | Solvent (v/v) | | | |
|---|---|---|---|---|---|
| | | | | | D |
| | | A | B | C | Acetone/n- |
| # | Acid | IPA/H₂O (4:1) | EtOAc | MTBE | Heptane (1:15) |
| 0 | Blank | Free Base | Amorphous* | Amorphous* | Amorphous |
| | | Type A | | | |
| 1 | HCl | Amorphous^{#} | HCl salt Type | HCl salt Type | HCl salt Type |
| | | | A | B | B |
| 2 | H₂SO₄ | Free Base | Amorphous | Amorphous | Amorphous |
| | | Type A | | | |
| 3 | H₃PO₄ | Gel | Phosphate | Phosphate | Phosphate |
| | | | Type A | Type A | Type A |
| 4 | Maleic acid | Amorphous^{#} | Amorphous | Maleate Type | Maleic acid |
| | | | | A | (one peak) |
| 5 | L-tartaric acid | Gel | L-tartrate Type | Amorphous | L-tartaric acid |
| | | | A | | |
| 6 | Fumaric acid | Fumarate | Fumarate | Fumarate | Fumarate |
| | | Type A | Type A | Type A | Type A + |
| | | | | | Fumaric acid |
| 7 | Citric acid | Gel | Amorphous | Amorphous + | Citric acid |
| | | | | peaks | |
| 8 | Glycolic acid | Glycolate Type | Glycolate Type | Glycolate Type | Glycolate Type |
| | | A | A + B | B | A |
| 9 | L-Malic acid | Amorphous* | Amorphous | Amorphous | Amorphous |
| 10 | Hippuric acid | Amorphous* | Hippuric acid | Hippuric acid | Hippuric acid |
| 11 | DL-Lactic acid | Lactate Type A | Lactate Type A | Lactate Type A | Lactate Type A |
| 12 | Adipic acid | Adipate Type A | Adipate Type | Adipate Type | Adipate Type |
| | | | B | B | A + Adipic acid |
| 13 | Acetic acid | Amorphous* | Acetate Type A | Acetate Type A | Acetate Type A |
| 14 | L-Ascorbic acid | Gel | L-Ascorbic | L-Ascorbic | L-Ascorbic |
| | | | acid | acid | acid |
| 15 | Succinic acid | Succinate | Succinate | Succinate | Succinate |
| | | Type A | Type A | Type A + | Type A + |
| | | | | Succinic acid | Succinic acid |
| 16 | L-Aspartic acid | Free Base | L-Aspartic acid | L-Aspartic acid | L-Aspartic acid |
| | | Type A + | | | |
| | | peaks | | | |
| 17 | Malonic acid | Amorphous* | Malonate Type | Amorphous | Malonate Type |
| | | | A | | B |
| 18 | Gentisic acid | Amorphous* | Gentisate Type | Gentisate Type | Weakly |
| | | | A | B | crystalline |
| 19 | Ethane-1.2- | Edisylate Type | Edisylate Type | Edisylate Type | Edisylate Type |
| | disulfonic acid | A | A | B | A |
| 20 | Benzoic acid | Amorphous* | Amorphous* | Amorphous* | Benzoate Type |
| | | | | | A |
| 21 | Ethanesulfonic | Esylate Type A | Esylate Type A | Esylate Type A | Esylate Type A |
| | acid | | | | |
| 22 | Methanesulfonic | Mesylate Type | Mesylate Type | Mesylate Type | Mesylate Type |
| | acid | A^{#} | A | A | A |
| 23 | Benzenesulfonic | Amorphous^{#} | Weakly | Amorphous | Besylate Type |
| | acid | | crystalline | | A |
| 24 | p- | Tosylate Type | Tosylate Type | Tosylate Type | Tosylate Type |
| | Toluenesulfonic | A^{#} | A | A | A |
| | acid | | | | |
| 25 | Oxalic acid | Oxalate Type | Oxalate Type | Oxalate Type | Weakly |
| | | A | B | B | crystalline |

^{#}Clear solution was obtained after slurry at RT. Anti-solvent MTBE was added for precipitation.
*Clear solution was obtained after adding anti-solvent MTBE, which was transferred to evaporate at RT.

TABLE 13
Purity summary of crystalline hits for 3-DM (3-DCR = 3-deuterocorynantheidine).

| | | Area (%) | | | | | | | |
|---|---|---|---|---|---|---|---|---|---|
| | | A | | B | | C | | D | |
| # | Acid | 3-DCR | 3-DM | 3-DCR | 3-DM | 3-DCR | 3-DM | 3-DCR | 3-DM |
| 0 | Blank | 1.01 | 98.63 | — | — | — | — | — | — |
| 1 | HCl | — | — | 2.62 | 96.22 | 2.02 | 94.59 | 2.46 | 92.60 |
| 2 | H₂SO₄ | 1.41 | 98.14 | — | — | — | — | — | — |
| 3 | H₃PO₄ | — | — | 2.26 | 97.29 | 2.22 | 97.33 | 2.55 | 96.06 |
| 4 | Maleic acid | — | — | — | — | 2.67 | 96.21 | — | — |
| 5 | L-tartaric acid | — | — | 2.52 | 96.89 | — | — | 2.23 | 96.50 |
| 6 | Fumaric acid | 2.53 | 96.81 | 2.64 | 96.48 | 2.67 | 96.25 | — | — |
| 7 | Citric acid | — | — | — | — | — | — | 2.28 | 95.75 |
| 8 | Glycolic acid | 0.84 | 98.90 | 2.29 | 97.45 | 2.39 | 97.13 | 3.13 | 95.49 |
| 9 | L-Malic acid | — | — | — | — | — | — | — | — |
| 10 | Hippuric acid | — | — | — | — | — | — | — | — |
| 11 | DL-Lactic acid | 1.16 | 98.55 | 2.47 | 96.55 | 2.59 | 96.20 | 2.69 | 96.14 |
| 12 | Adipic acid | 1.89 | 97.45 | 2.08 | 97.07 | 2.46 | 96.49 | — | — |
| 13 | Acetic acid | — | — | 0.76 | 99.05 | 1.37 | 98.21 | 1.75 | 97.42 |
| 14 | L-Ascorbic acid | — | — | — | — | — | — | — | — |
| 15 | Succinic acid | 1.96 | 97.66 | 2.47 | 96.90 | — | — | — | — |
| 16 | L-Aspartic acid | — | — | 2.84 | 96.69 | 2.66 | 96.14 | 2.22 | 96.44 |
| 17 | Malonic acid | — | — | — | — | 2.51 | 96.39 | 2.40 | 96.69 |
| 18 | Gentisic acid | — | — | 2.32 | 97.16 | 2.33 | 97.04 | 1.72 | 97.51 |
| 19 | Ethane-1.2- | 0.89 | 98.75 | 2.05 | 95.15 | 2.47 | 87.63 | 2.44 | 91.05 |
| | disulfonic acid | | | | | | | | |
| 20 | Benzoic acid | — | — | 2.50 | 96.62 | — | — | 2.25 | 96.98 |
| 21 | Ethanesulfonic | 1.61 | 97.44 | 2.22 | 97.10 | 1.89 | 97.39 | 1.85 | 96.97 |
| | acid | | | | | | | | |
| 22 | Methanesulfonic | 2.68 | 95.94 | 1.57 | 98.11 | 2.41 | 96.48 | 2.33 | 92.68 |
| | acid | | | | | | | | |
| 23 | Benzenesulfonic | — | — | — | — | — | — | 2.49 | 94.92 |
| | acid | | | | | | | | |
| 24 | p- | 2.29 | 96.99 | 2.57 | 93.90 | 2.28 | 96.26 | 2.58 | 94.60 |
| | Toluenesulfonic | | | | | | | | |
| | acid | | | | | | | | |
| 25 | Oxalic acid | 3.07 | 96.42 | 2.61 | 95.98 | 2.62 | 96.39 | — | — |

TABLE 14
Characterization results of salt hits for 3-DM.

| Salt Hit | TGA Weight loss | DSC endotherm | Molar ratio |
|---|---|---|---|
| (Sample ID) | (%, temp.) | (peak, ° C.) | (acid/free base) |
| HCl salt Type A | 2.6 (to 150° C.) | 202.2, 262.2 | 1.1:1 |
| (810397-04-B1) | | | |
| HCl salt Type B | 9.3 (to 175° C.) | 69.5, 198.3, 256.4 | 1.3:1 |
| (810397-04-D1) | | | |
| Phosphate Type A | 7.9 (to 200° C.) | 72.4, 178.1, | 1.2:1 |
| (810397-04-D3) | | 194.3, 232.2 | |
| Maleate Type A | 10.8 (to 150° C.) | 91.0, 168.9, 212.2 | 1.0:1 |
| (810397-04-C4) | | | |
| L-Tartrate Type A | 5.9 (to 150° C.) | 96.1, 146.4, 158.6 | 1.0:1 |
| (810397-04-B5) | | | |
| Fumarate Type A | 3.6 (to 150° C.) | 256.4 | 1.0:1 |
| (810397-04-C6) | | | |
| Glycolate Type A | 3.6 (to 150° C.) | 216.9 | 1.1:1 |
| (810397-04-D8) | | | |
| Glycolate Type B | 1.9 (to 150° C.) | 69.1, 126.0, 220.8 | 1.1:1 |
| (810397-04-C8) | | | |
| Lactate Type A | 1.5 (to 150° C.) | 221.5 | 1.1:1 |
| (810397-04-B11) | | | |
| Adipate Type A | 5.0 (to 170° C.) | 212.0 | 0.9:1 |
| (810397-04-A12) | | | |
| Adipate Type B | 4.9 (to 170° C.) | 211.1 | 1.0:1 |
| (810397-04-C12) | | | |
| Acetate Type A | 13.0 (to 180° C.) | 152.3 | 1.2:1 |
| (810397-04-D13) | | | |
| Succinate Type A | 1.4 (to 120° C.) | 201.2 | 1.1:1 |
| (810397-04-B15) | | | |
| Malonate Type A | 3.6 (to 110° C.) | 110.7, 154.4 | 1.2:1 |
| (810397-04-B17) | | | |
| Malonate Type B | 3.3 (to 100° C.) | 79.2, 109.7, 155.7 | 2.0:1 |
| (810397-04-D17) | | | |
| Gentisate Type A | 8.8 (to 200° C.) | 182.4 | 1.0:1 |
| (810397-04-B18) | | | |
| Gentisate Type B | 8.8 (to 200° C.) | 171.9, 179.5 | 1.1:1 |
| (810397-04-C18) | | | |
| Edisylate Type A | 16.7 (to 200° C.) | 69.2, 148.2, 259.1 | 0.5:1 |
| (810397-04-A19) | | | |
| Edisylate Type B | 16.0 (to 200° C.) | 70.7, 167.3, | 0.6:1 |
| (810397-04-C19) | | 191.5, 225.2 | |
| Benzoate Type A | 9.8 (to 130° C.) | 108.5, 212.6, 241.2 | 1:1 |
| (810397-04-D20) | | | |
| Esylate Type A | 2.5 (to 150° C.) | 230.0, 269.7 | 0.9:1 |
| (810397-04-C21) | | | |
| Mesylate Type A | 2.2 (to 150° C.) | 271.9 | 1.0:1 |
| (810397-04-C22) | | | |
| Besylate Type A | 7.0 (to 170° C.) | 144.4, 261.6 | 0.9:1 |
| (810397-04-D23) | | | |
| Tosylate Type A | 8.0 (to 170° C.) | 95.2, 234.0 | 1.0:1 |
| (810397-04-B24) | | | |
| Oxalate Type A | 6.2 (to 150° C.) | 82.0, 240.8 | 0.8:1 |
| (810397-04-A25) | | | |
| Oxalate Type B | 6.0 (to 150° C.) | 243.7 | 1.1:1 |
| (810397-04-B25) | | | |
| Free Base Type A | 11.7 (to 110° C.) | 61.8, 80.6 | — |
| (810397-04-A0) | | | |

XRPD results for 3-DM salt leads are provided below in Table 15 and the respective XRPD traces are shown in FIGS. 4 to 8.

TABLE 15
XRPD copper radiation results of salt leads for 3-DM.

| | Pos. | Height | FWHM | | Rel. Int. |
|---|---|---|---|---|---|
| 3-DM Salt | [°2θ] | [cts] | Left [°2θ] | d-spacing | [%] |
| Fumarate | 6.23 | 466.62 | 0.1279 | 14.19 | 3.06 |
| Type A | 8.41 | 15233.33 | 0.1279 | 10.51 | 100.00 |
| (FIG. 4) | 9.58 | 1168.41 | 0.0768 | 9.23 | 7.67 |
| | 13.43 | 337.14 | 0.0768 | 6.60 | 2.21 |
| | 14.40 | 327.55 | 0.1279 | 6.15 | 2.15 |
| | 15.62 | 223.48 | 0.1023 | 5.67 | 1.47 |
| | 16.16 | 300.94 | 0.1023 | 5.49 | 1.98 |
| | 16.85 | 508.24 | 0.1023 | 5.26 | 3.34 |
| | 17.46 | 2895.93 | 0.1279 | 5.08 | 19.01 |
| | 18.68 | 456.62 | 0.1279 | 4.75 | 3.00 |
| | 19.20 | 2583.93 | 0.1279 | 4.62 | 16.96 |
| | 21.59 | 1167.87 | 0.1279 | 4.12 | 7.67 |
| | 22.35 | 293.44 | 0.1023 | 3.98 | 1.93 |
| | 23.03 | 871.16 | 0.1023 | 3.86 | 5.72 |
| | 23.35 | 350.40 | 0.1023 | 3.81 | 2.30 |
| | 23.75 | 496.99 | 0.1279 | 3.75 | 3.26 |
| | 25.36 | 1302.70 | 0.2047 | 3.51 | 8.55 |
| | 25.77 | 2088.21 | 0.1535 | 3.46 | 13.71 |
| | 26.96 | 454.35 | 0.1279 | 3.31 | 2.98 |
| | 28.94 | 481.81 | 0.1279 | 3.09 | 3.16 |
| | 31.11 | 892.87 | 0.1279 | 2.87 | 5.86 |
| | 32.82 | 223.25 | 0.1791 | 2.73 | 1.47 |
| | 34.04 | 153.07 | 0.1535 | 2.63 | 1.00 |
| | 37.97 | 37.54 | 0.3070 | 2.37 | 0.25 |
| L-Lactate | 6.86 | 912.47 | 0.1023 | 12.88 | 41.23 |
| Type A | 9.96 | 2213.14 | 0.1023 | 8.88 | 100.00 |
| (FIG. 5) | 10.73 | 383.13 | 0.0768 | 8.24 | 17.31 |
| | 11.05 | 1598.46 | 0.1023 | 8.01 | 72.23 |
| | 13.02 | 279.14 | 0.1023 | 6.80 | 12.61 |
| | 13.80 | 135.41 | 0.1023 | 6.42 | 6.12 |
| | 15.74 | 1336.33 | 0.1023 | 5.63 | 60.38 |
| | 17.74 | 684.95 | 0.1279 | 5.00 | 30.95 |
| | 18.11 | 126.02 | 0.1535 | 4.90 | 5.69 |
| | 18.80 | 239.00 | 0.0768 | 4.72 | 10.80 |
| | 19.29 | 732.83 | 0.1023 | 4.60 | 33.11 |
| | 19.77 | 501.70 | 0.1023 | 4.49 | 22.67 |
| | 20.63 | 1665.56 | 0.1279 | 4.31 | 75.26 |
| | 22.26 | 1777.09 | 0.1279 | 3.99 | 80.30 |
| | 23.62 | 162.88 | 0.2047 | 3.77 | 7.36 |
| | 24.38 | 294.07 | 0.1279 | 3.65 | 13.29 |
| | 24.75 | 1351.16 | 0.1023 | 3.60 | 61.05 |
| | 26.96 | 266.73 | 0.1023 | 3.31 | 12.05 |
| | 27.18 | 294.90 | 0.1023 | 3.28 | 13.33 |
| | 28.01 | 105.83 | 0.1535 | 3.19 | 4.78 |
| | 29.30 | 186.15 | 0.1023 | 3.05 | 8.41 |
| | 31.22 | 245.17 | 0.1023 | 2.86 | 11.08 |
| | 33.81 | 111.51 | 0.1791 | 2.65 | 5.04 |
| | 35.59 | 60.07 | 0.4093 | 2.52 | 2.71 |
| Glycolate | 7.06 | 2750.57 | 0.0768 | 12.53 | 48.83 |
| Type A | 10.12 | 5633.51 | 0.1023 | 8.74 | 100.00 |
| (FIG. 6) | 11.25 | 3840.63 | 0.0768 | 7.87 | 68.17 |
| | 13.18 | 448.79 | 0.1023 | 6.72 | 7.97 |
| | 14.11 | 670.06 | 0.0768 | 6.28 | 11.89 |
| | 15.04 | 93.51 | 0.1535 | 5.89 | 1.66 |
| | 15.97 | 2524.11 | 0.1023 | 5.55 | 44.81 |
| | 17.99 | 1120.78 | 0.1023 | 4.93 | 19.89 |
| | 18.11 | 988.75 | 0.0768 | 4.90 | 17.55 |
| | 18.51 | 291.60 | 0.1023 | 4.79 | 5.18 |
| | 18.87 | 171.68 | 0.1023 | 4.70 | 3.05 |
| | 19.19 | 375.37 | 0.1023 | 4.62 | 6.66 |
| | 19.49 | 1396.21 | 0.1023 | 4.55 | 24.78 |
| | 19.69 | 863.13 | 0.0768 | 4.51 | 15.32 |
| | 20.34 | 1027.37 | 0.1023 | 4.37 | 18.24 |
| | 20.88 | 3514.53 | 0.1023 | 4.25 | 62.39 |
| | 22.58 | 2504.54 | 0.1279 | 3.94 | 44.46 |
| | 23.24 | 183.27 | 0.1023 | 3.83 | 3.25 |
| | 23.72 | 214.78 | 0.1279 | 3.75 | 3.81 |
| | 24.02 | 259.94 | 0.1023 | 3.71 | 4.61 |
| | 24.90 | 417.41 | 0.1279 | 3.58 | 7.41 |
| | 25.16 | 2177.96 | 0.1023 | 3.54 | 38.66 |
| | 27.60 | 777.81 | 0.1279 | 3.23 | 13.81 |
| | 28.27 | 106.24 | 0.1535 | 3.16 | 1.89 |
| | 29.55 | 140.41 | 0.1535 | 3.02 | 2.49 |
| | 30.15 | 281.33 | 0.1535 | 2.96 | 4.99 |
| | 30.59 | 189.47 | 0.1279 | 2.92 | 3.36 |
| | 31.61 | 447.05 | 0.1023 | 2.83 | 7.94 |
| | 32.24 | 128.20 | 0.1535 | 2.78 | 2.28 |
| | 34.18 | 121.06 | 0.1535 | 2.62 | 2.15 |
| Succinate | 6.23 | 495.96 | 0.0768 | 14.19 | 3.58 |
| Type A | 8.46 | 13846.33 | 0.1023 | 10.45 | 100.00 |
| (FIG. 7) | 9.62 | 919.89 | 0.1023 | 9.20 | 6.64 |
| | 10.06 | 187.37 | 0.1023 | 8.79 | 1.35 |
| | 14.35 | 804.02 | 0.1023 | 6.17 | 5.81 |
| | 15.65 | 585.24 | 0.1023 | 5.66 | 4.23 |
| | 16.12 | 599.70 | 0.1023 | 5.50 | 4.33 |
| | 16.94 | 382.32 | 0.0768 | 5.23 | 2.76 |
| | 17.56 | 2944.00 | 0.1023 | 5.05 | 21.26 |
| | 18.72 | 326.94 | 0.0768 | 4.74 | 2.36 |
| | 19.27 | 2840.79 | 0.1279 | 4.61 | 20.52 |
| | 21.19 | 166.60 | 0.1535 | 4.19 | 1.20 |
| | 21.71 | 1024.55 | 0.1279 | 4.09 | 7.40 |
| | 22.33 | 378.95 | 0.1023 | 3.98 | 2.74 |
| | 23.13 | 984.83 | 0.1023 | 3.84 | 7.11 |
| | 23.73 | 395.60 | 0.1279 | 3.75 | 2.86 |
| | 24.48 | 113.21 | 0.1535 | 3.64 | 0.82 |
| | 25.03 | 318.43 | 0.1023 | 3.56 | 2.30 |
| | 25.34 | 647.53 | 0.0768 | 3.52 | 4.68 |
| | 25.53 | 1081.82 | 0.1023 | 3.49 | 7.81 |
| | 25.94 | 1289.20 | 0.1279 | 3.44 | 9.31 |
| | 26.98 | 297.64 | 0.1535 | 3.30 | 2.15 |
| | 29.08 | 270.84 | 0.1023 | 3.07 | 1.96 |
| | 30.54 | 135.43 | 0.1535 | 2.93 | 0.98 |
| | 31.27 | 845.96 | 0.1279 | 2.86 | 6.11 |
| | 32.99 | 168.07 | 0.1279 | 2.72 | 1.21 |
| | 34.25 | 105.34 | 0.1535 | 2.62 | 0.76 |
| Mesylate | 6.67 | 2264.45 | 0.1279 | 13.26 | 100.00 |
| Type A | 8.18 | 238.23 | 0.0768 | 10.81 | 10.52 |
| (FIG. 8) | 10.00 | 169.74 | 0.1023 | 8.85 | 7.50 |
| | 11.56 | 728.33 | 0.1279 | 7.66 | 32.16 |
| | 13.34 | 524.16 | 0.1279 | 6.64 | 23.15 |
| | 14.93 | 504.38 | 0.1023 | 5.94 | 22.27 |
| | 15.29 | 241.58 | 0.1279 | 5.79 | 10.67 |
| | 16.69 | 1058.47 | 0.1279 | 5.31 | 46.74 |
| | 17.34 | 941.15 | 0.1535 | 5.12 | 41.56 |
| | 18.60 | 593.38 | 0.1279 | 4.77 | 26.20 |
| | 18.89 | 715.15 | 0.1535 | 4.70 | 31.58 |
| | 19.76 | 357.10 | 0.1023 | 4.49 | 15.77 |
| | 20.03 | 700.95 | 0.1279 | 4.43 | 30.95 |
| | 21.13 | 190.26 | 0.1535 | 4.20 | 8.40 |
| | 21.42 | 211.16 | 0.1535 | 4.15 | 9.33 |
| | 22.21 | 242.58 | 0.1279 | 4.00 | 10.71 |
| | 22.72 | 308.31 | 0.1023 | 3.91 | 13.62 |
| | 23.69 | 187.94 | 0.1023 | 3.76 | 8.30 |
| | 24.40 | 295.39 | 0.1535 | 3.65 | 13.04 |
| | 25.08 | 208.62 | 0.1791 | 3.55 | 9.21 |
| | 25.97 | 465.61 | 0.1791 | 3.43 | 20.56 |
| | 26.92 | 117.37 | 0.3070 | 3.31 | 5.18 |
| | 28.51 | 96.14 | 0.2047 | 3.13 | 4.25 |
| | 32.80 | 52.09 | 0.3070 | 2.73 | 2.30 |

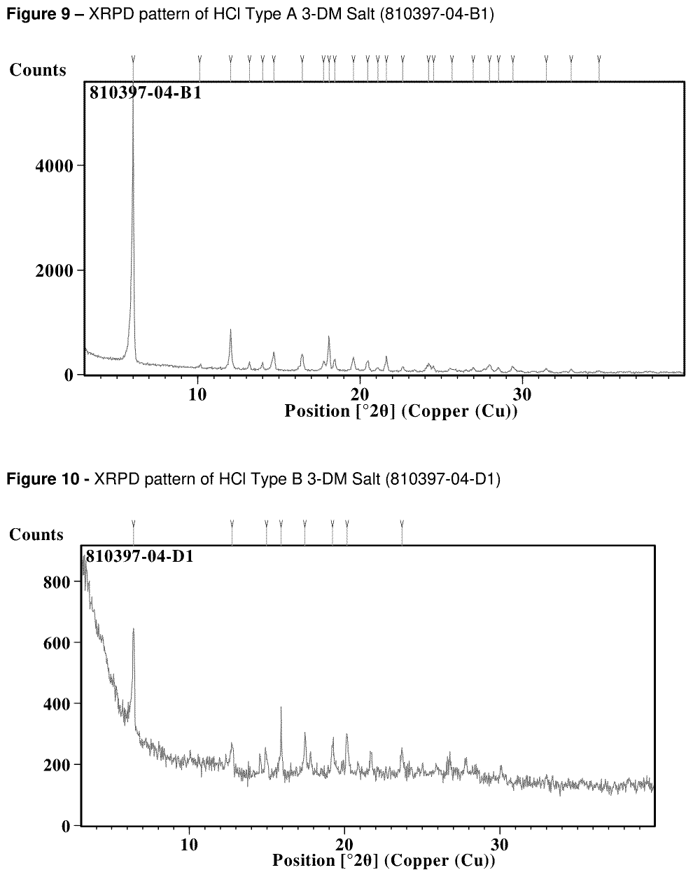

XRPD data for the other 3-DM salt forms made and characterized are listed in Table 16 and their respective XRPD traces are shown in FIGS. 9 to 29.

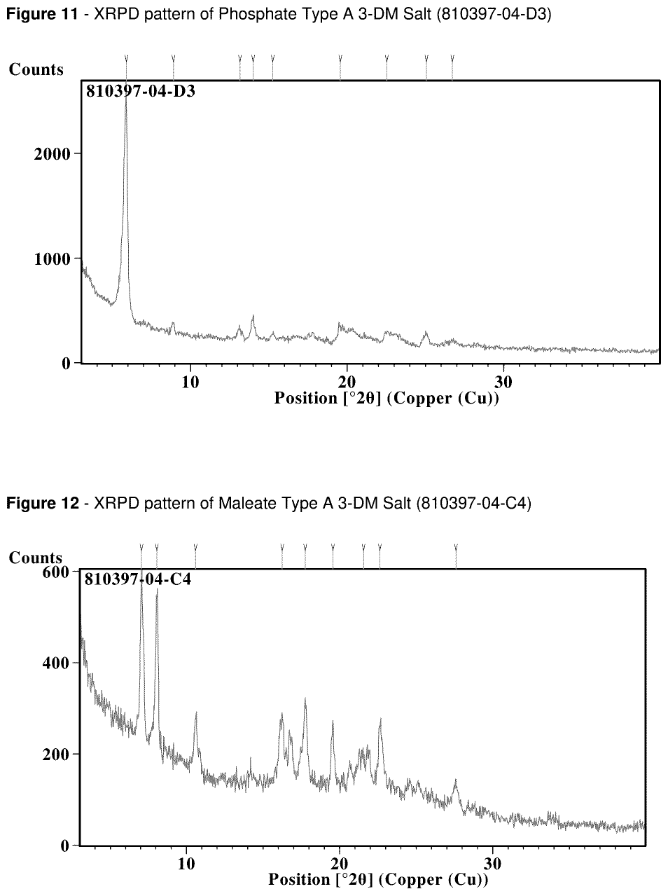

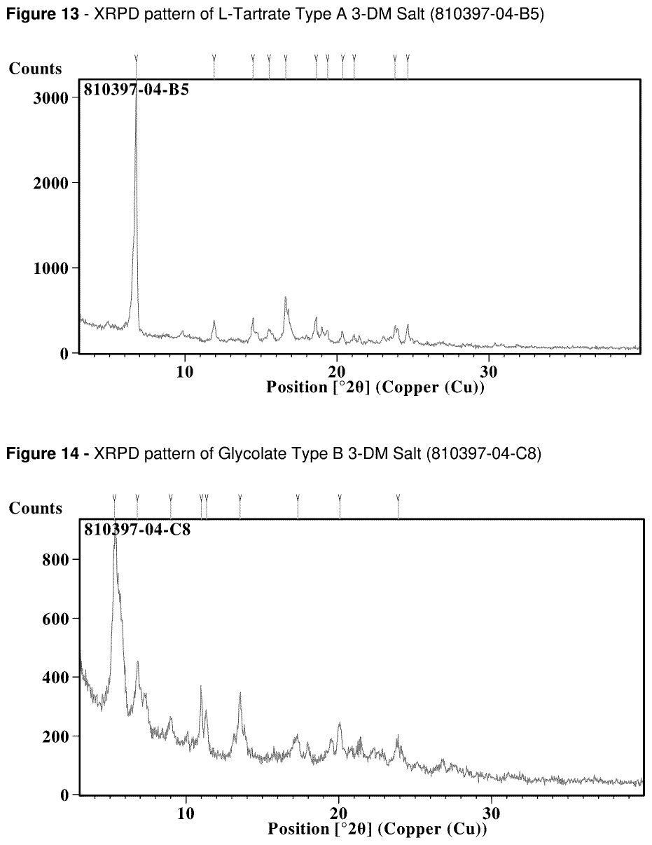

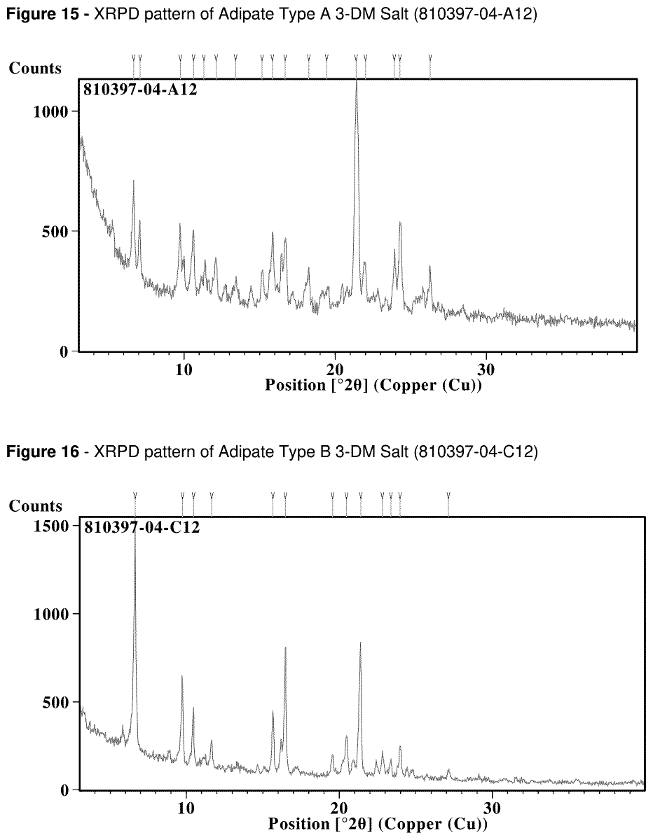

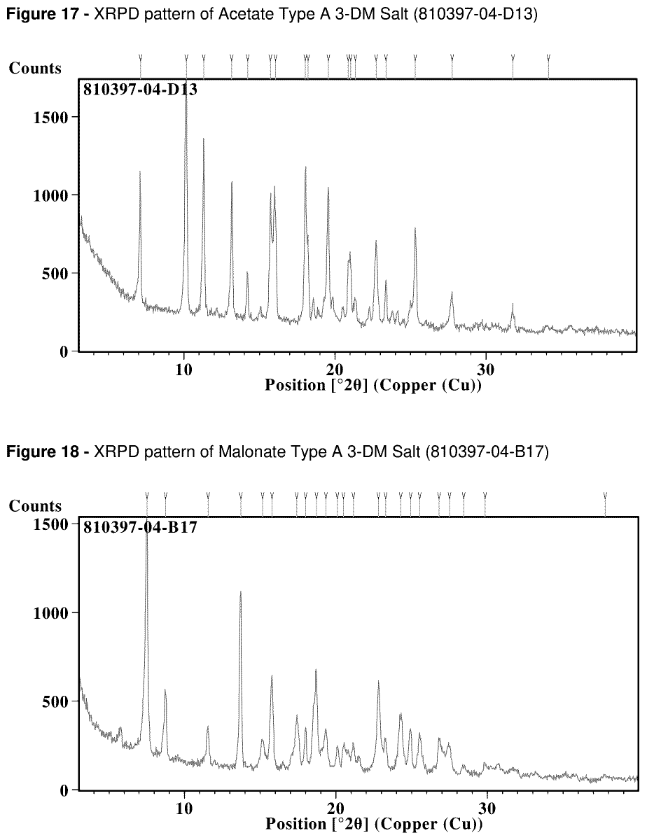

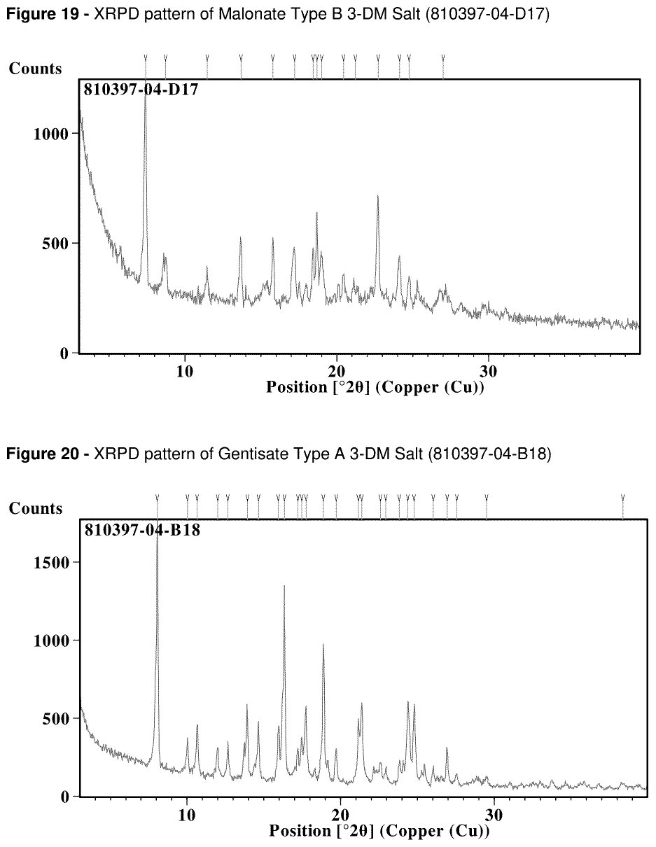

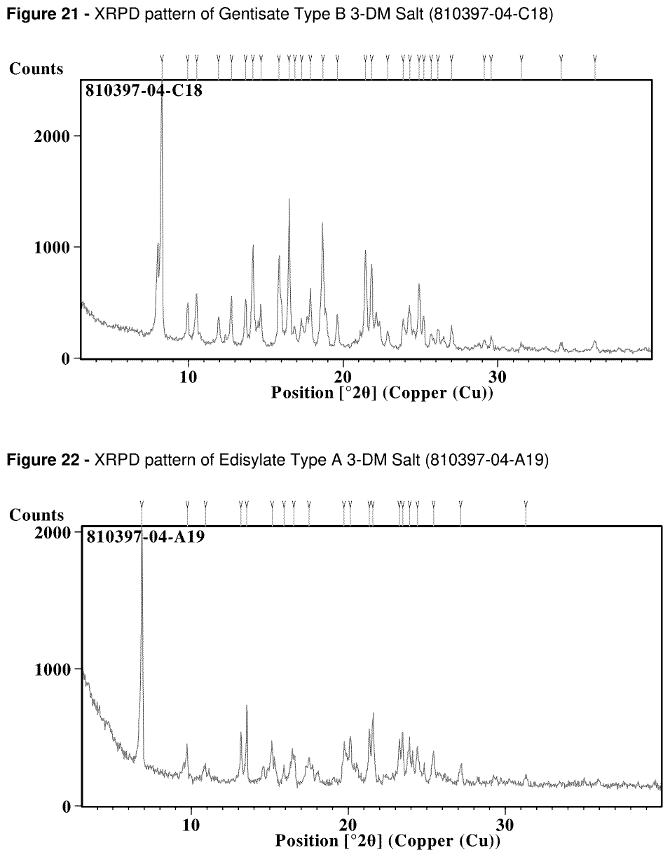

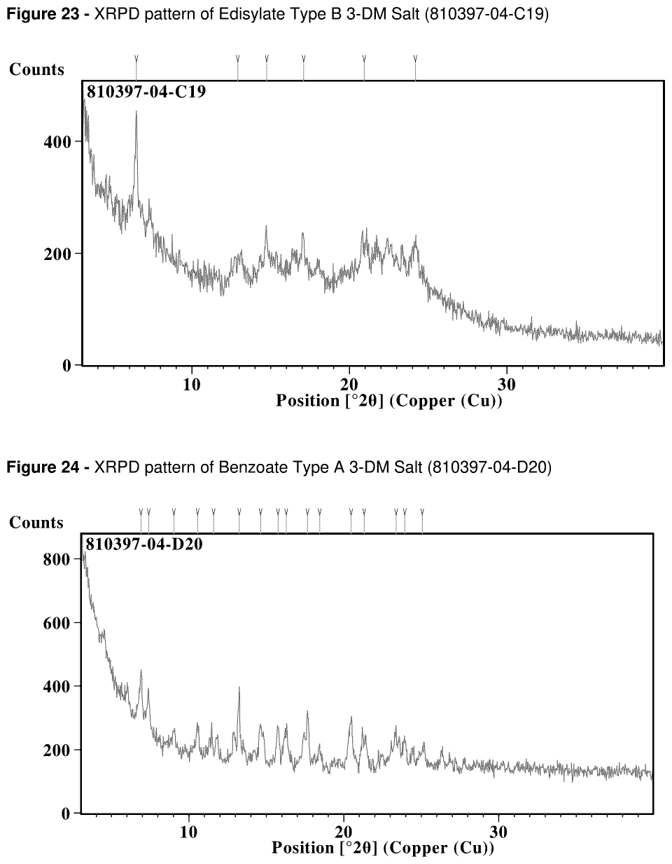

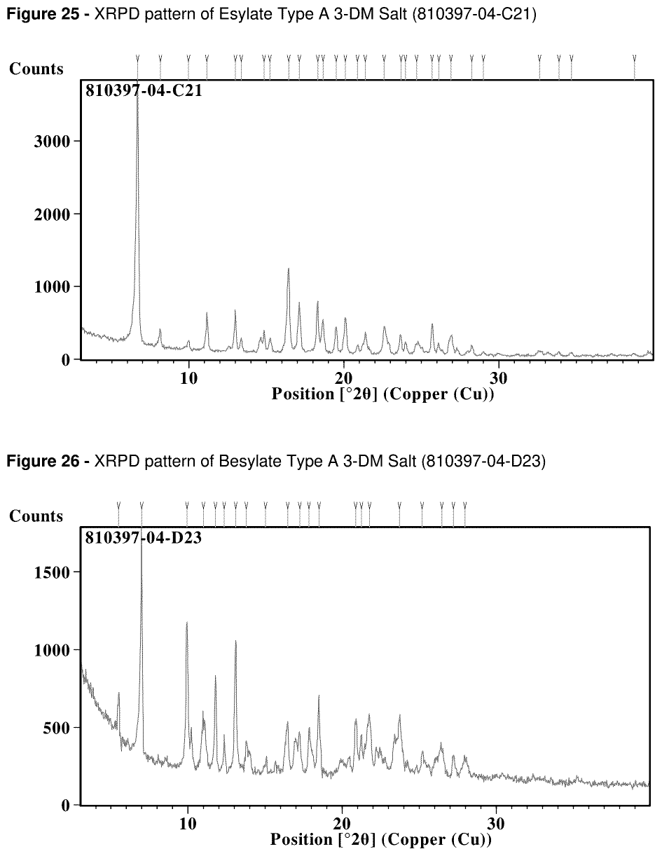

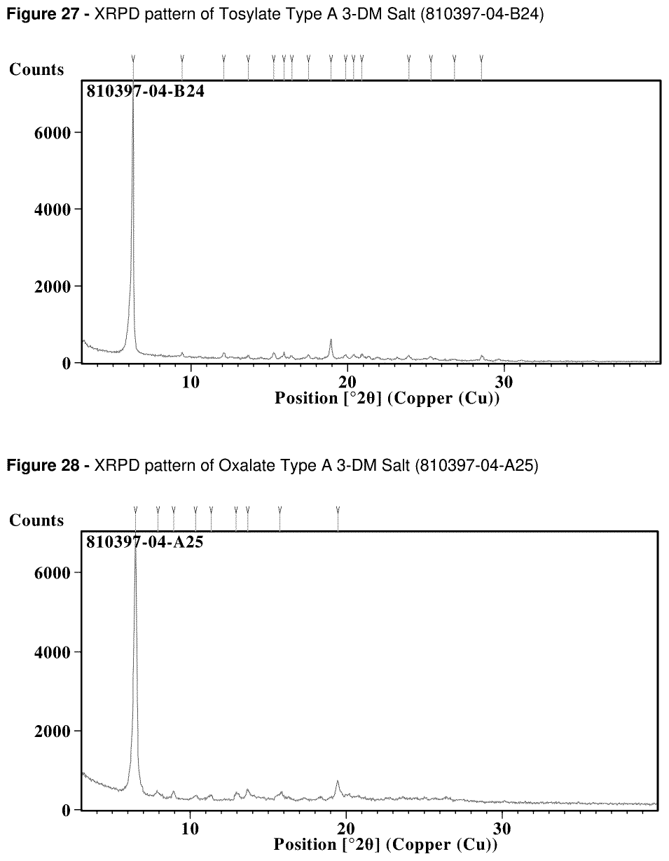

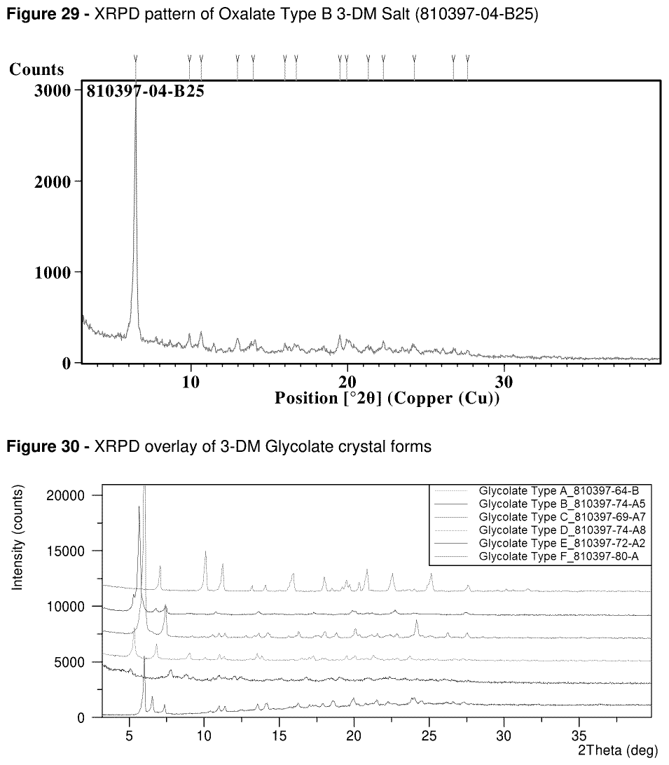

TABLE 16
XRPD copper radiation results of other salts for 3-DM.

| | | | FWHM | | Rel. |
|---|---|---|---|---|---|
| | Pos. | Height | Left | d-spacing | Int. |
| 3-DM Salt | [°2θ] | [cts] | [°2θ] | [Å] | [%] |
| HCl Type A | 6.01 | 5471.24 | 0.1023 | 14.69 | 100.00 |
| (810397-04- | 10.11 | 36.23 | 0.3070 | 8.75 | 0.66 |
| B1) | 12.03 | 770.84 | 0.0768 | 7.36 | 14.09 |
| (FIG. 9) | 13.19 | 139.55 | 0.1023 | 6.71 | 2.55 |
| | 13.98 | 136.40 | 0.1023 | 6.34 | 2.49 |
| | 14.69 | 342.73 | 0.1279 | 6.03 | 6.26 |
| | 16.43 | 313.97 | 0.1279 | 5.39 | 5.74 |
| | 17.76 | 180.39 | 0.1279 | 5.00 | 3.30 |
| | 18.08 | 665.54 | 0.1023 | 4.91 | 12.16 |
| | 18.42 | 220.46 | 0.1023 | 4.82 | 4.03 |
| | 19.58 | 254.85 | 0.1535 | 4.53 | 4.66 |
| | 20.48 | 201.58 | 0.1535 | 4.34 | 3.68 |
| | 21.07 | 63.97 | 0.1535 | 4.22 | 1.17 |
| | 21.63 | 288.45 | 0.1279 | 4.11 | 5.27 |
| | 22.63 | 88.17 | 0.2047 | 3.93 | 1.61 |
| | 24.22 | 141.32 | 0.2047 | 3.67 | 2.58 |
| | 24.53 | 92.47 | 0.1279 | 3.63 | 1.69 |
| | 25.64 | 51.08 | 0.3070 | 3.47 | 0.93 |
| | 26.98 | 84.57 | 0.1535 | 3.30 | 1.55 |
| | 27.98 | 139.09 | 0.2047 | 3.19 | 2.54 |
| | 28.53 | 84.66 | 0.1535 | 3.13 | 1.55 |
| | 29.41 | 105.78 | 0.2047 | 3.04 | 1.93 |
| | 31.46 | 75.16 | 0.2047 | 2.84 | 1.37 |
| HCl Type B | 6.40 | 322.11 | 0.1023 | 13.82 | 100.00 |
| (810397-04- | 12.73 | 75.53 | 0.3070 | 6.95 | 23.45 |
| D1) | 14.95 | 61.29 | 0.1535 | 5.93 | 19.03 |
| (FIG. 10) | 15.90 | 225.46 | 0.0768 | 5.57 | 69.99 |
| | 17.44 | 123.71 | 0.1535 | 5.09 | 38.40 |
| | 19.22 | 96.49 | 0.2047 | 4.62 | 29.95 |
| | 20.16 | 115.59 | 0.1535 | 4.40 | 35.89 |
| | 23.68 | 64.14 | 0.3070 | 3.76 | 19.91 |
| Phosphate | 5.89 | 2174.99 | 0.1791 | 15.02 | 100.00 |
| Type A | 8.88 | 92.40 | 0.1535 | 9.96 | 4.25 |
| (810397-04- | 13.14 | 93.55 | 0.3070 | 6.74 | 4.30 |
| D3) | 13.98 | 214.05 | 0.2047 | 6.34 | 9.84 |
| (FIG. 11) | 15.24 | 53.48 | 0.3070 | 5.81 | 2.46 |
| | 19.57 | 146.86 | 0.4093 | 4.54 | 6.75 |
| | 22.52 | 113.58 | 0.3070 | 3.95 | 5.22 |
| | 25.07 | 102.83 | 0.4093 | 3.55 | 4.73 |
| | 26.71 | 42.05 | 0.6140 | 3.34 | 1.93 |
| Maleate | 7.05 | 369.14 | 0.1023 | 12.54 | 100.00 |
| Type A | 8.06 | 325.32 | 0.1535 | 10.97 | 88.13 |
| (810397-04- | 10.58 | 119.23 | 0.1535 | 8.36 | 32.30 |
| C4 | 16.23 | 145.05 | 0.3070 | 5.46 | 39.30 |
| (FIG. 12) | 17.75 | 188.43 | 0.1535 | 5.00 | 51.04 |
| | 19.54 | 145.00 | 0.1791 | 4.54 | 39.28 |
| | 21.57 | 65.58 | 0.8187 | 4.12 | 17.77 |
| | 22.63 | 148.06 | 0.2558 | 3.93 | 40.11 |
| | 27.58 | 50.71 | 0.3070 | 3.23 | 13.74 |
| L-Tartrate | 6.78 | 3012.28 | 0.1279 | 13.04 | 100.00 |
| Type A | 11.89 | 233.63 | 0.1279 | 7.44 | 7.76 |
| (810397-04- | 14.46 | 282.46 | 0.1023 | 6.12 | 9.38 |
| B5) | 15.51 | 138.47 | 0.2047 | 5.71 | 4.60 |
| (FIG. 13) | 16.60 | 537.37 | 0.1023 | 5.34 | 17.84 |
| | 18.62 | 302.16 | 0.1279 | 4.77 | 10.03 |
| | 19.35 | 141.27 | 0.1535 | 4.59 | 4.69 |
| | 20.36 | 131.67 | 0.1535 | 4.36 | 4.37 |
| | 21.11 | 101.91 | 0.2047 | 4.21 | 3.38 |
| | 23.82 | 202.50 | 0.3070 | 3.74 | 6.72 |
| | 24.65 | 234.43 | 0.1279 | 3.61 | 7.78 |
| Glycolate | 5.31 | 559.43 | 0.2047 | 16.65 | 100.00 |
| Type B | 6.81 | 207.66 | 0.2047 | 12.98 | 37.12 |
| (810397-04- | 8.98 | 63.50 | 0.3070 | 9.85 | 11.35 |
| C8 | 10.99 | 184.46 | 0.1023 | 8.05 | 32.97 |
| (FIG. 14) | 11.32 | 127.38 | 0.1535 | 7.82 | 22.77 |
| | 13.53 | 211.79 | 0.2047 | 6.55 | 37.86 |
| | 17.32 | 66.45 | 0.4093 | 5.12 | 11.88 |
| | 20.05 | 115.12 | 0.2558 | 4.43 | 20.58 |
| | 23.88 | 60.26 | 0.5117 | 3.73 | 10.77 |
| Adipate | 6.64 | 375.12 | 0.0768 | 13.31 | 40.46 |
| Type A | 7.07 | 232.49 | 0.1279 | 12.51 | 25.08 |
| (810397-04- | 9.73 | 294.49 | 0.1023 | 9.09 | 31.76 |
| A12) | 10.61 | 269.69 | 0.1023 | 8.34 | 29.09 |
| (FIG. 15) | 11.29 | 103.80 | 0.4093 | 7.83 | 11.20 |
| | 12.10 | 167.05 | 0.1535 | 7.31 | 18.02 |
| | 13.40 | 82.24 | 0.3070 | 6.61 | 8.87 |
| | 15.16 | 135.71 | 0.1535 | 5.84 | 14.64 |
| | 15.84 | 309.04 | 0.1023 | 5.59 | 33.33 |
| | 16.70 | 272.88 | 0.1791 | 5.31 | 29.43 |
| | 18.23 | 144.67 | 0.1535 | 4.87 | 15.60 |
| | 19.44 | 68.42 | 0.5117 | 4.57 | 7.38 |
| | 21.38 | 927.12 | 0.1279 | 4.16 | 100.00 |
| | 21.99 | 187.93 | 0.2047 | 4.04 | 20.27 |
| | 23.91 | 227.45 | 0.1535 | 3.72 | 24.53 |
| | 24.29 | 372.75 | 0.1535 | 3.66 | 40.21 |
| | 26.27 | 183.35 | 0.2047 | 3.39 | 19.78 |
| Adipate | 6.64 | 1344.11 | 0.1023 | 13.32 | 100.00 |
| Type B | 9.74 | 486.46 | 0.1023 | 9.09 | 36.19 |
| (810397-04- | 10.44 | 314.32 | 0.1023 | 8.47 | 23.39 |
| C12) | 11.64 | 149.61 | 0.0768 | 7.60 | 11.13 |
| (FIG. 16) | 15.66 | 342.03 | 0.1023 | 5.66 | 25.45 |
| | 16.47 | 739.71 | 0.0768 | 5.38 | 55.03 |
| | 19.57 | 118.26 | 0.1279 | 4.54 | 8.80 |
| | 20.47 | 224.99 | 0.1535 | 4.34 | 16.74 |
| | 21.39 | 691.20 | 0.1279 | 4.15 | 51.42 |
| | 22.81 | 141.34 | 0.1023 | 3.90 | 10.52 |
| | 23.36 | 88.24 | 0.1535 | 3.81 | 6.56 |
| | 23.98 | 177.26 | 0.1279 | 3.71 | 13.19 |
| | 27.14 | 52.73 | 0.2047 | 3.29 | 3.92 |
| Acetate | 7.10 | 841.05 | 0.1023 | 12.46 | 57.38 |
| Type A | 10.14 | 1465.74 | 0.1535 | 8.72 | 100.00 |
| (810397-04- | 11.30 | 1153.88 | 0.1023 | 7.83 | 78.72 |
| D13) | 13.16 | 873.36 | 0.1023 | 6.73 | 59.59 |
| (FIG. 17) | 14.20 | 294.00 | 0.1023 | 6.24 | 20.06 |
| | 15.72 | 802.23 | 0.0768 | 5.64 | 54.73 |
| | 16.04 | 708.13 | 0.1791 | 5.52 | 48.31 |
| | 18.03 | 991.88 | 0.1023 | 4.92 | 67.67 |
| | 18.22 | 503.56 | 0.0768 | 4.87 | 34.36 |
| | 19.55 | 862.57 | 0.1279 | 4.54 | 58.85 |
| | 20.87 | 380.61 | 0.1279 | 4.26 | 25.97 |
| | 21.02 | 396.45 | 0.0768 | 4.23 | 27.05 |
| | 21.35 | 158.28 | 0.1535 | 4.16 | 10.80 |
| | 22.72 | 540.35 | 0.2047 | 3.91 | 36.87 |
| | 23.39 | 274.39 | 0.1023 | 3.80 | 18.72 |
| | 25.33 | 632.04 | 0.1535 | 3.52 | 43.12 |
| | 27.74 | 231.17 | 0.1535 | 3.22 | 15.77 |
| | 31.77 | 130.77 | 0.2047 | 2.82 | 8.92 |
| | 34.12 | 26.88 | 0.6140 | 2.63 | 1.83 |
| Malonate | 7.51 | 1325.29 | 0.1279 | 11.77 | 100.00 |
| Type A | 8.74 | 350.43 | 0.1279 | 10.12 | 26.44 |
| (810397-04- | 11.56 | 191.83 | 0.1535 | 7.66 | 14.47 |
| B17) | 13.70 | 991.43 | 0.1279 | 6.46 | 74.81 |
| (FIG. 18) | 15.13 | 156.86 | 0.2047 | 5.85 | 11.84 |
| | 15.78 | 522.99 | 0.2047 | 5.62 | 39.46 |
| | 17.39 | 286.23 | 0.1535 | 5.10 | 21.60 |
| | 18.01 | 223.95 | 0.1023 | 4.93 | 16.90 |
| | 18.70 | 558.37 | 0.1023 | 4.75 | 42.13 |
| | 19.32 | 229.70 | 0.1791 | 4.59 | 17.33 |
| | 20.10 | 131.16 | 0.1535 | 4.42 | 9.90 |
| | 20.51 | 145.23 | 0.1535 | 4.33 | 10.96 |
| | 21.15 | 145.63 | 0.1535 | 4.20 | 10.99 |
| | 22.82 | 507.87 | 0.1791 | 3.90 | 38.32 |
| | 23.28 | 176.20 | 0.1535 | 3.82 | 13.30 |
| | 24.28 | 303.51 | 0.2558 | 3.67 | 22.90 |
| | 24.95 | 223.65 | 0.2047 | 3.57 | 16.88 |
| | 25.54 | 210.44 | 0.2047 | 3.49 | 15.88 |
| | 26.81 | 197.95 | 0.2047 | 3.33 | 14.94 |
| | 27.48 | 153.28 | 0.2558 | 3.25 | 11.57 |
| | 28.44 | 40.35 | 0.3070 | 3.14 | 3.04 |
| | 29.85 | 53.29 | 0.3070 | 2.99 | 4.02 |
| | 37.80 | 24.41 | 0.6140 | 2.38 | 1.84 |
| Malonate | 7.393905 | 911.630700 | 0.153504 | 11.95636 | 100.00 |
| Type B | 8.705001 | 129.308500 | 0.307008 | 10.15829 | 14.18 |
| (810397-04- | 11.443480 | 133.246600 | 0.204672 | 7.73277 | 14.62 |
| D17) | 13.679360 | 261.793200 | 0.153504 | 6.47348 | 28.72 |
| (FIG. 19) | 15.786770 | 298.816000 | 0.127920 | 5.61375 | 32.78 |
| | 17.205240 | 247.311000 | 0.281424 | 5.15399 | 27.13 |
| | 18.427380 | 260.767600 | 0.076752 | 4.81483 | 28.60 |
| | 18.675700 | 426.904700 | 0.076752 | 4.75137 | 46.83 |
| | 18.991190 | 238.203700 | 0.204672 | 4.67314 | 26.13 |
| | 20.427050 | 151.108200 | 0.153504 | 4.34779 | 16.58 |
| | 21.201710 | 85.153030 | 0.307008 | 4.19065 | 9.34 |
| | 22.711740 | 515.768900 | 0.153504 | 3.91533 | 56.58 |
| | 24.125140 | 246.694100 | 0.179088 | 3.68905 | 27.06 |
| | 24.744300 | 144.154700 | 0.204672 | 3.59813 | 15.81 |
| | 27.009250 | 84.520030 | 0.614016 | 3.30132 | 9.27 |
| Gentisate | 8.06 | 1582.31 | 0.1023 | 10.97 | 100.00 |
| Type A | 10.03 | 225.27 | 0.1023 | 8.82 | 14.24 |
| (810397-04- | 10.66 | 309.03 | 0.1023 | 8.30 | 19.53 |
| B18) | 11.98 | 181.06 | 0.1023 | 7.39 | 11.44 |
| (FIG. 20) | 12.65 | 221.15 | 0.1023 | 7.00 | 13.98 |
| | 13.91 | 470.72 | 0.1023 | 6.37 | 29.75 |
| | 14.65 | 359.01 | 0.1023 | 6.05 | 22.69 |
| | 15.94 | 317.32 | 0.1279 | 5.56 | 20.05 |
| | 16.34 | 1170.18 | 0.0768 | 5.42 | 73.95 |
| | 17.20 | 203.02 | 0.0768 | 5.16 | 12.83 |
| | 17.45 | 274.73 | 0.0768 | 5.08 | 17.36 |
| | 17.73 | 474.84 | 0.1023 | 5.00 | 30.01 |
| | 18.88 | 881.85 | 0.1023 | 4.70 | 55.73 |
| | 19.71 | 210.08 | 0.1279 | 4.50 | 13.28 |
| | 21.15 | 398.05 | 0.0768 | 4.20 | 25.16 |
| | 21.37 | 513.08 | 0.0768 | 4.16 | 32.43 |
| | 22.60 | 130.17 | 0.1535 | 3.93 | 8.23 |
| | 22.94 | 91.47 | 0.1535 | 3.88 | 5.78 |
| | 23.81 | 126.46 | 0.1279 | 3.74 | 7.99 |
| | 24.38 | 527.21 | 0.1791 | 3.65 | 33.32 |
| | 24.79 | 510.89 | 0.1535 | 3.59 | 32.29 |
| | 26.03 | 108.51 | 0.1791 | 3.42 | 6.86 |
| | 26.93 | 229.84 | 0.1023 | 3.31 | 14.53 |
| | 27.55 | 70.69 | 0.2047 | 3.24 | 4.47 |
| | 29.51 | 51.86 | 0.3070 | 3.03 | 3.28 |
| | 38.37 | 30.75 | 0.3070 | 2.35 | 1.94 |
| Gentisate | 8.26 | 2365.32 | 0.0768 | 10.71 | 100.00 |
| Type B | 9.94 | 322.72 | 0.1023 | 8.90 | 13.64 |
| (810397-04- | 10.52 | 437.18 | 0.1023 | 8.41 | 18.48 |
| C18) | 11.94 | 234.71 | 0.1023 | 7.41 | 9.92 |
| (FIG. 21) | 12.76 | 419.68 | 0.1023 | 6.94 | 17.74 |
| | 13.68 | 403.02 | 0.1023 | 6.47 | 17.04 |
| | 14.16 | 890.33 | 0.1279 | 6.26 | 37.64 |
| | 14.67 | 349.21 | 0.0768 | 6.04 | 14.76 |
| | 15.84 | 799.58 | 0.1279 | 5.59 | 33.80 |
| | 16.50 | 1311.53 | 0.1023 | 5.37 | 55.45 |
| | 16.86 | 157.48 | 0.1535 | 5.26 | 6.66 |
| | 17.30 | 218.60 | 0.1535 | 5.13 | 9.24 |
| | 17.88 | 510.62 | 0.1023 | 4.96 | 21.59 |
| | 18.67 | 1101.93 | 0.1023 | 4.75 | 46.59 |
| | 19.63 | 285.81 | 0.1279 | 4.52 | 12.08 |
| | 21.43 | 884.16 | 0.1279 | 4.15 | 37.38 |
| | 21.84 | 741.10 | 0.1279 | 4.07 | 31.33 |
| | 22.88 | 136.29 | 0.1535 | 3.89 | 5.76 |
| | 23.87 | 253.19 | 0.1279 | 3.73 | 10.70 |
| | 24.31 | 316.14 | 0.1791 | 3.66 | 13.37 |
| | 24.91 | 578.38 | 0.1279 | 3.57 | 24.45 |
| | 25.20 | 287.42 | 0.1279 | 3.53 | 12.15 |
| | 25.69 | 125.67 | 0.1535 | 3.47 | 5.31 |
| | 26.13 | 157.32 | 0.1535 | 3.41 | 6.65 |
| | 26.99 | 187.12 | 0.1279 | 3.30 | 7.91 |
| | 29.14 | 79.31 | 0.1535 | 3.06 | 3.35 |
| | 29.56 | 121.70 | 0.1279 | 3.02 | 5.15 |
| | 31.53 | 58.79 | 0.1535 | 2.84 | 2.49 |
| | 34.09 | 65.23 | 0.2047 | 2.63 | 2.76 |
| | 36.28 | 90.02 | 0.2558 | 2.48 | 3.81 |
| Edisylate | 6.84 | 1772.74 | 0.1023 | 12.91 | 100.00 |
| Type A | 9.73 | 239.23 | 0.1023 | 9.09 | 13.49 |
| (810397-04- | 10.89 | 62.52 | 0.3070 | 8.12 | 3.53 |
| A19) | 13.15 | 358.17 | 0.1023 | 6.73 | 20.20 |
| (FIG. 22) | 13.53 | 553.52 | 0.0768 | 6.54 | 31.22 |
| | 15.14 | 295.09 | 0.1023 | 5.85 | 16.65 |
| | 15.89 | 108.43 | 0.1535 | 5.58 | 6.12 |
| | 16.52 | 192.88 | 0.2558 | 5.37 | 10.88 |
| | 17.50 | 146.03 | 0.4093 | 5.07 | 8.24 |
| | 19.72 | 254.81 | 0.1535 | 4.50 | 14.37 |
| | 20.13 | 335.61 | 0.0768 | 4.41 | 18.93 |
| | 21.34 | 387.88 | 0.0768 | 4.16 | 21.88 |
| | 21.57 | 453.85 | 0.1279 | 4.12 | 25.60 |
| | 23.26 | 306.91 | 0.1023 | 3.82 | 17.31 |
| | 23.46 | 366.60 | 0.0768 | 3.79 | 20.68 |
| | 23.89 | 332.69 | 0.1023 | 3.72 | 18.77 |
| | 24.40 | 258.49 | 0.1023 | 3.65 | 14.58 |
| | 25.43 | 229.38 | 0.1279 | 3.50 | 12.94 |
| | 27.17 | 125.77 | 0.1535 | 3.28 | 7.09 |
| | 31.33 | 63.59 | 0.2047 | 2.86 | 3.59 |
| Edisylate | 6.47 | 190.15 | 0.1279 | 13.67 | 100.00 |
| Type B | 12.89 | 39.88 | 0.6140 | 6.87 | 20.97 |
| (810397-04- | 14.75 | 72.81 | 0.3070 | 6.01 | 38.29 |
| C19) | 17.08 | 67.53 | 0.3070 | 5.19 | 35.51 |
| (FIG. 23) | 20.92 | 30.55 | 0.4093 | 4.25 | 16.06 |
| | 24.18 | 56.49 | 0.3070 | 3.68 | 29.71 |
| Benzoate | 6.88 | 137.63 | 0.1535 | 12.84 | 59.95 |
| Type A | 7.38 | 112.52 | 0.2047 | 11.98 | 49.02 |
| (810397-04- | 9.02 | 50.52 | 0.3070 | 9.81 | 22.01 |
| D20) | 10.55 | 90.55 | 0.1535 | 8.39 | 39.45 |
| (FIG. 24) | 11.59 | 40.77 | 0.4093 | 7.64 | 17.76 |
| | 13.23 | 229.56 | 0.1279 | 6.69 | 100.00 |
| | 14.60 | 113.18 | 0.2047 | 6.07 | 49.30 |
| | 15.74 | 118.47 | 0.1535 | 5.63 | 51.61 |
| | 16.28 | 105.42 | 0.3070 | 5.45 | 45.92 |
| | 17.66 | 169.37 | 0.1279 | 5.02 | 73.78 |
| | 18.44 | 50.63 | 0.3070 | 4.81 | 22.06 |
| | 20.45 | 148.57 | 0.2047 | 4.34 | 64.72 |
| | 21.30 | 85.90 | 0.4093 | 4.17 | 37.42 |
| | 23.38 | 86.26 | 0.4093 | 3.80 | 37.58 |
| | 23.93 | 81.57 | 0.1535 | 3.72 | 35.53 |
| | 25.07 | 48.36 | 0.3070 | 3.55 | 21.07 |
| Esylate Type | 6.67 | 3667.35 | 0.1279 | 13.25 | 100.00 |
| A (810397- | 8.13 | 218.87 | 0.1023 | 10.87 | 5.97 |
| 04-C21) | 9.97 | 111.78 | 0.1535 | 8.87 | 3.05 |
| (FIG. 25) | 11.16 | 521.74 | 0.1023 | 7.93 | 14.23 |
| | 12.98 | 556.17 | 0.1023 | 6.82 | 15.17 |
| | 13.37 | 183.35 | 0.1279 | 6.62 | 5.00 |
| | 14.85 | 292.74 | 0.1023 | 5.97 | 7.98 |
| | 15.21 | 183.06 | 0.1279 | 5.82 | 4.99 |
| | 16.42 | 1158.26 | 0.1279 | 5.40 | 31.58 |
| | 17.12 | 696.45 | 0.1279 | 5.18 | 18.99 |
| | 18.30 | 711.73 | 0.1023 | 4.85 | 19.41 |
| | 18.65 | 450.76 | 0.1279 | 4.76 | 12.29 |
| | 19.50 | 357.56 | 0.1279 | 4.55 | 9.75 |
| | 20.10 | 485.39 | 0.1535 | 4.42 | 13.24 |
| | 20.87 | 105.87 | 0.1535 | 4.26 | 2.89 |
| | 21.38 | 293.58 | 0.1279 | 4.16 | 8.01 |
| | 22.59 | 374.76 | 0.1279 | 3.94 | 10.22 |
| | 23.67 | 260.88 | 0.1279 | 3.76 | 7.11 |
| | 23.97 | 166.36 | 0.1535 | 3.71 | 4.54 |
| | 24.70 | 141.85 | 0.2558 | 3.60 | 3.87 |
| | 25.69 | 431.64 | 0.1535 | 3.47 | 11.77 |
| | 26.12 | 157.28 | 0.1023 | 3.41 | 4.29 |
| | 26.89 | 248.50 | 0.2558 | 3.32 | 6.78 |
| | 28.25 | 134.62 | 0.1535 | 3.16 | 3.67 |
| | 29.00 | 52.72 | 0.2047 | 3.08 | 1.44 |
| | 32.62 | 57.06 | 0.3070 | 2.75 | 1.56 |
| | 33.89 | 44.97 | 0.2047 | 2.65 | 1.23 |
| | 34.69 | 41.22 | 0.2047 | 2.59 | 1.12 |
| | 38.74 | 25.68 | 0.3070 | 2.32 | 0.70 |
| Besylate | 5.49 | 254.56 | 0.0768 | 16.11 | 17.38 |
| Type A | 6.98 | 1464.97 | 0.1023 | 12.67 | 100.00 |
| (810397-04- | 9.92 | 936.45 | 0.1023 | 8.92 | 63.92 |
| D23) | 11.00 | 299.09 | 0.3070 | 8.05 | 20.42 |
| (FIG. 26) | 11.78 | 600.93 | 0.1023 | 7.51 | 41.02 |
| | 12.35 | 225.11 | 0.1023 | 7.17 | 15.37 |
| | 13.09 | 838.56 | 0.1023 | 6.77 | 57.24 |
| | 13.77 | 192.66 | 0.1279 | 6.43 | 13.15 |
| | 15.01 | 52.71 | 0.3070 | 5.90 | 3.60 |
| | 16.47 | 286.20 | 0.1023 | 5.38 | 19.54 |
| | 17.24 | 244.41 | 0.1023 | 5.14 | 16.68 |
| | 17.85 | 294.68 | 0.1023 | 4.97 | 20.12 |
| | 18.48 | 503.28 | 0.1023 | 4.80 | 34.35 |
| | 20.88 | 328.01 | 0.1535 | 4.25 | 22.39 |
| | 21.24 | 245.26 | 0.1023 | 4.18 | 16.74 |
| | 21.76 | 373.87 | 0.2558 | 4.08 | 25.52 |
| | 23.72 | 372.74 | 0.1279 | 3.75 | 25.44 |
| | 25.20 | 155.89 | 0.1535 | 3.53 | 10.64 |
| | 26.46 | 175.45 | 0.3070 | 3.37 | 11.98 |
| | 27.21 | 131.25 | 0.1535 | 3.28 | 8.96 |
| | 27.98 | 119.10 | 0.3070 | 3.19 | 8.13 |
| Tosylate | 6.29 | 7162.69 | 0.1023 | 14.06 | 100.00 |
| Type A | 9.42 | 102.41 | 0.1535 | 9.39 | 1.43 |
| (810397-04- | 12.09 | 134.52 | 0.1535 | 7.32 | 1.88 |
| B24) | 13.63 | 67.80 | 0.1535 | 6.49 | 0.95 |
| (FIG. 27) | 15.27 | 160.36 | 0.1535 | 5.80 | 2.24 |
| | 15.92 | 161.50 | 0.1023 | 5.57 | 2.25 |
| | 16.42 | 76.74 | 0.1535 | 5.40 | 1.07 |
| | 17.48 | 105.68 | 0.1535 | 5.07 | 1.48 |
| | 18.93 | 524.42 | 0.1023 | 4.69 | 7.32 |
| | 19.88 | 109.82 | 0.2047 | 4.47 | 1.53 |
| | 20.37 | 119.82 | 0.2047 | 4.36 | 1.67 |
| | 20.91 | 135.14 | 0.1535 | 4.25 | 1.89 |
| | 23.89 | 119.27 | 0.2047 | 3.72 | 1.67 |
| | 25.30 | 93.55 | 0.1535 | 3.52 | 1.31 |
| | 26.82 | 26.25 | 0.6140 | 3.32 | 0.37 |
| | 28.53 | 118.19 | 0.1791 | 3.13 | 1.65 |
| Oxalate | 6.49 | 6650.19 | 0.1535 | 13.62 | 100.00 |
| Type A | 7.93 | 100.70 | 0.3070 | 11.15 | 1.51 |
| (810397-04- | 8.92 | 181.89 | 0.1535 | 9.91 | 2.74 |
| A25) | 10.32 | 85.94 | 0.3070 | 8.57 | 1.29 |
| (FIG. 28) | 11.33 | 103.40 | 0.2047 | 7.81 | 1.55 |
| | 12.92 | 167.51 | 0.3070 | 6.85 | 2.52 |
| | 13.68 | 269.32 | 0.2047 | 6.47 | 4.05 |
| | 15.75 | 162.38 | 0.3070 | 5.63 | 2.44 |
| | 19.45 | 453.55 | 0.1535 | 4.56 | 6.82 |
| Oxalate | 6.47 | 2862.81 | 0.1023 | 13.66 | 100.00 |
| Type B | 9.90 | 151.67 | 0.1023 | 8.94 | 5.30 |
| (810397-04- | 10.63 | 183.15 | 0.1279 | 8.32 | 6.40 |
| B25) | 12.96 | 139.44 | 0.1791 | 6.83 | 4.87 |
| (FIG. 29) | 13.95 | 84.95 | 0.4093 | 6.35 | 2.97 |
| | 16.00 | 90.27 | 0.1535 | 5.54 | 3.15 |
| | 16.72 | 80.26 | 0.3070 | 5.30 | 2.80 |
| | 19.50 | 206.28 | 0.1279 | 4.55 | 7.21 |
| | 19.93 | 157.42 | 0.2047 | 4.46 | 5.50 |
| | 21.32 | 75.05 | 0.3070 | 4.17 | 2.62 |
| | 22.28 | 140.88 | 0.1535 | 3.99 | 4.92 |
| | 24.25 | 104.26 | 0.3070 | 3.67 | 3.64 |
| | 26.75 | 62.82 | 0.3070 | 3.33 | 2.19 |
| | 27.65 | 59.74 | 0.3070 | 3.23 | 2.09 |

### Example 3. Re-Preparation and Characterization of 3-Deuteromitragynine Salt Leads

Based on the solid-state characterization of 3-DM salts in Example 2, Fumarate Type A, Glycolate Type A, L-Lactate Type A, Succinate Type A and Mesylate Type A were selected as salt leads of 3-DM for scale up and further evaluation. Detailed re-preparation procedures of the salt leads are listed in Table 17. XRPD comparison results confirmed all salt leads were generated on scale up. Characterization results of re-prepared salt leads are summarized in Table 18.

TABLE 17
Preparation procedures for 3-DM salt leads.

| 3-DM Salt | | |
|---|---|---|
| (Sample ID) | Procedure | |
| Fumarate Type A | 1. | Weigh 199.4 mg of free base (810397-01-A) and 58.9 mg |
| (810397-28-B1) | | of fumaric acid into a 20-mL glass vial. |
| | 2. | Add 5 mL IPA/H₂O (4:1, v/v) to form a suspension. |
| | | Magnetically stir (~1000 rpm) at RT for 39 hrs. |
| | 3. | Isolate suspension by centrifugation (10000 rpm, 2 min). |
| | | Dry solids at 50° C. under vacuum for ~7 hrs. |
| | 4. | Collect solids, a total of 232.8 mg of material was obtained |
| | | (Yield: 90.1%). |
| Fumarate Type A | 1. | Weigh 200.6 mg of free base (810397-01-A) and 59.1 mg |
| (810397-36-B1) | | of fumaric acid into a 20-mL glass vial. |
| | 2. | Add 5 mL IPA/H₂O (4:1, v/v) to form a suspension. |
| | | Magnetically stir (~1000 rpm) at RT for 48 hrs. |
| | 3. | Isolate solids by centrifugation (10000 rpm, 2 min). Dry |
| | | solids at 50° C. under vacuum for ~7 hrs. Collect solids. |
| Glycolate Type A | 1. | Weigh 300.2 mg of free base (810397-01-A) and 58.2 mg |
| (810397-28-B2) | | of glycolic acid into a 20-mL glass vial. |
| | 2. | Add 5 mL IPA/H₂O (4:1, v/v) to form a suspension. |
| | | Magnetically stir (~1000 rpm) at RT for 39 hrs. |
| | 3. | Isolate solids by centrifugation (10000 rpm, 2 min). Dry |
| | | solids at 50° C. under vacuum for ~7 hrs. |
| | 4. | Collect solids, a total of 230.5 mg of material was obtained |
| | | (Yield: 64.3%). |
| Glycolate Type A | 1. | Weigh 299.8 mg of free base (810397-01-A) and 58.3 mg |
| (810397-36-B2) | | of glycolic acid into a 20-mL glass vial. |
| | 2. | Add 5 mL IPA/H₂O (4:1, v/v) to form a suspension. |
| | | Magnetically stir (~1000 rpm) at RT for 48 hrs. |
| | 3. | Isolate solids by centrifugation (10000 rpm, 2 min). Dry |
| | | solids at 50° C. under vacuum for ~7 hrs. Collect solids. |
| L-Lactate Type A | 1. | Weigh 55.0 mg of L-lactic acid into a 20-mL glass vial, add |
| (810397-28-B3) | | 5 mL IPA/H₂O (4:1, v/v) to dissolve acid. |
| | 2. | Weigh 200.3 mg of free base (810397-01-A) into the acid |
| | | solution. Magnetically stir (~1000 rpm) at RT for 39 hrs. |
| | 3. | Isolate solids by centrifugation (10000 rpm, 2 min). Dry |
| | | solids at 50° C. under vacuum for ~7 hrs. |
| | 4. | Collect solids, a total of 138.3 mg of material was obtained |
| | | (Yield: 54.2%). |
| L-Lactate Type A | 1. | Weigh 83.1 mg of L-Lactic acid into a 20-mL glass vial, add |
| (810397-36-B3) | | 5 mL IPA/H₂O (4:1, v/v) to dissolve acid. |
| | 2. | Weigh 300.3 mg of free base (810397-01-A) into the acid |
| | | solution. Magnetically stir (~1000 rpm) at RT for 48 hrs. |
| | 3. | Isolate solids by centrifugation (10000 rpm, 2 min). Dry |
| | | solids at 50° C. under vacuum for ~7 hrs. Collect solids. |
| Succinate Type A | 1. | Weigh 199.6 mg of free base (810397-01-A) and 60.3 mg |
| (810397-28-B4) | | of succinic acid into a 20-mL glass vial. |
| | 2. | Add 5 mL IPA/H₂O (4:1, v/v) to form a suspension. |
| | | Magnetically stir (~1000 rpm) at RT for 39 hrs. |
| | 3. | Isolate solids by centrifugation (10000 rpm, 2 min). Dry |
| | | solids at 50° C. under vacuum for ~7 hrs. |
| | 4. | Collect solids, a total of 179.2 mg of material was obtained |
| | | (Yield: 68.9%). |
| Succinate Type A | 1. | Weigh 200.5 mg of free base (810397-01-A) and 60.7 mg |
| (810397-36-B4) | | of succinic acid into a 20-mL glass vial. |
| | 2. | Add 5 mL IPA/H₂O (4:1, v/v) to form a suspension. |
| | | Magnetically stir (~1000 rpm) at RT for 48 hrs. |
| | 3. | Isolate solids by centrifugation (10000 rpm, 2 min). Dry |
| | | solids at 50° C. under vacuum for ~7 hrs. Collect solids. |
| Mesylate Type A | 1. | Weigh 79.2 mg of Methanesulfonic acid into a 20-mL glass |
| (810397-28-B5) | | vial, add 5 mL EtOAc to dissolve acid. |
| | 2. | Weigh 326.6 mg of free base (810397-01-A) into the acid |
| | | solution. Magnetically stir (~1000 rpm) at RT for 39 hrs. |
| | 3. | Isolate solids by centrifugation (10000 rpm, 2 min). Dry |
| | | solids at 50° C. under vacuum for ~7 hrs. |
| | 4. | Collect solids, a total of 378.5 mg of material was obtained |
| | | (Yield: 93.3%). |

TABLE 18
Characterization results of re-prepared 3-DM salt leads.

| | TGA | | | Water | | |
|---|---|---|---|---|---|---|
| | weight | DSC | Molar | content | | |
| 3-DM Salt | loss | endotherm | ratio | by KF | Purity | 3-DCR |
| (Sample ID) | (%) | (° C., onset) | (acid/base) | (%) | (area %) | (area %) |
| Fumarate | 0.3 | 256.1 | 1:1 | 0.71 | 96.89 | 2.51 |
| Type A | (to 150° | | | | | |
| (810397- | C.) | | | | | |
| 28-B1) | | | | | | |
| Fumarate | 1.6 | 255.2 | 1:1 | — | 97.03 | 2.45 |
| Type A | (to 150° | | | | | |
| (810397- | C.) | | | | | |
| 36-B1) | | | | | | |
| Glycolate | 0.5 | 212.1 | 1:1 | 0.43 | 98.69 | 1.10 |
| Type A | (to 150° | | | | | |
| (810397- | C.) | | | | | |
| 28-B2) | | | | | | |
| Glycolate | 1.1 | 212.1 | 1:1 | — | 97.53 | 1.93 |
| Type A | (to 150° | | | | | |
| (810397- | C.) | | | | | |
| 36-B2) | | | | | | |
| L-Lactate | 0.9 | 211.6 | 1:1 | 0.31 | 98.18 | 1.50 |
| Type A | (to 150° | | | | | |
| (810397- | C.) | | | | | |
| 28-B3) | | | | | | |
| L-Lactate | 1.9 | 224.2 | 0.9:1 | — | 98.82 | 0.94 |
| Type A | (to 150° | | | | | |
| (810397- | C.) | | | | | |
| 36-B3) | | | | | | |
| Succinate | 2.3 | 198.2*, | 1:1 | 0.41 | 97.92 | 1.87 |
| Type A | (to 150° | 202.3* | | | | |
| (810397- | C.) | | | | | |
| 28-B4) | | | | | | |
| Succinate | 1.1 | 198.0*, | 1.1:1 | — | 97.89 | 1.81 |
| Type A | (to 150° | 202.6* | | | | |
| (810397- | C.) | | | | | |
| 36-B4) | | | | | | |
| Mesylate | 1.3 | 266.4 | 1:1 | 0.50 | 96.48 | 2.65 |
| Type A | (to 150° | | | | | |
| (810397- | C.) | | | | | |
| 28-B5) | | | | | | |

*Peak temperature.

### Example 4: Solubility of 3-Deuteromitragynine Salt Leads

Solubility of 3-DM Fumarate Type A, Glycolate Type A, L-Lactate Type A, Succinate Type A, and Mesylate Type A was measured after 4 hrs at 37° C. in water and the following bio-relevant media: simulated gastric fluid (SGF), fasting-state simulated intestinal fluid (FaSSIF), and fed-state simulated intestinal fluid (FeSSIF). Amorphous free base was also assessed as a comparison.

Preparation of SGF. Weighed 49.5 mg of NaCl and 25.4 mg of Triton X-100 into a 100-mL volumetric flask. Added appropriate volume of purified water and sonicated until all solids were completely dissolved. Added about 1.632 mL of aq. HCl solution (1 M) and sufficient purified water to achieve the target volume and adjust to pH 1.8. The pH value was checked with a pH meter and found to be 1.83.

Preparation of FaSSIF Dissolving Buffer. Weighed 340.8 mg of NaH₂PO₄, 43.0 mg of NaOH, and 619.6 mg of NaCl into a 100-mL volumetric flask. Added appropriate volume of purified water and sonicated until all solids were completely dissolved. Added sufficient purified water to achieve the target volume and adjust to pH 6.5. The pH value was checked with a pH meter and found to be 6.54.

Preparation of FaSSIF. Weighed 110.4 mg of simulated intestinal fluid (SIF) powder into a 50-mL volumetric flask. Added appropriate volume of FaSSIF dissolving buffer and sonicated until SIF powder was completely dissolved. Then diluted to volume with FaSSIF dissolving buffer and mixed well. The FaSSIF solution was equilibrated to RT for 2 hrs before use.

Preparation of FeSSIF Dissolving Buffer. Weighed 0.82 mL of glacial acetic acid, 404.9 mg of NaOH, and 1188.2 mg of NaCl into a 100-mL volumetric flask. Added appropriate volume of purified water and sonicated until all solids were completely dissolved. Added sufficient purified water to achieve the target volume and adjust to pH 5.0. The pH value was checked with a pH meter and found to be 4.96.

Preparation of FeSSIF. Weighed 559.6 mg of simulated intestinal fluid (SIF) powder into a 50-mL volumetric flask. Added appropriate volume of FeSSIF dissolving buffer and sonicated until SIF powder was completely dissolved. Then diluted to volume with FeSSIF dissolving buffer and mixed well. The FeSSIF solution was equilibrated to RT for 2 hrs before use.

Solubility Measurement. Solids were suspended in selected media at a loading of 10 mg/ml (calculated based on free base). The suspensions were agitated on a rolling incubator at 25 rpm and 37° C., prior to sampling at 4 hrs. At this time, suspensions were separated by centrifugation (˜10,000 rpm, 37° C., 3 min) and the supernatants were filtered through 0.45 μm PTFE membranes. The pH of the filtrates was determined and they were analyzed by HPLC to determine solubility. Residual solids were analyzed by XRPD to determine their crystal form. Results are summarized in Table 19.

All salt leads showed higher solubility than amorphous free base in all media, except for Fumarate Type A in SGF. Glycolate Type A showed the highest solubility in SGF. XRPD overlay of the residual solids from solubility samples showed that no form change occurred for any of the tested salt forms or amorphous free base in any medium after 4 h. However, for Fumarate Type A, Glycolate Type A, and amorphous free base, an additional peak at 8.9° was observed in the XRPD spectra of the residual solids from FeSSIF experiments, which may result from sodium acetate.

TABLE 19
Summary of solubility results for 3-DM salts.

| Salt lead | H₂O | | | SGF | | | FaSSIF | | | FeSSIF | | |
|---|---|---|---|---|---|---|---|---|---|---|---|---|
| (810397-) | S | pH | FC | S | pH | FC | S | pH | FC | S | pH | FC |
| Fumarate | 0.30 | 3.4 | No | 1.07 | 1.9 | No | 2.35 | 5.6 | No | 1.49 | 4.9 | No |
| Type A | | | | | | | | | | | | |
| (36-B1) | | | | | | | | | | | | |
| Glycolate | 5.32 | 5.2 | No | 7.43 | 3.2 | No | 1.16 | 6.2 | No | 2.16 | 5.0 | No |
| Type A | | | | | | | | | | | | |
| (36-B2) | | | | | | | | | | | | |
| L-Lactate | 1.54 | 5.4 | No | 6.89 | 2.9 | No | 1.03 | 6.2 | No | 2.09 | 5.0 | No |
| Type A | | | | | | | | | | | | |
| (36-B3) | | | | | | | | | | | | |
| Succinate | 2.58 | 4.5 | No | 6.78 | 3.2 | No | 4.16 | 5.3 | No | 2.67 | 4.9 | No |
| Type A | | | | | | | | | | | | |
| (36-B4) | | | | | | | | | | | | |
| Mesylate | 13.1* | 3.2 | No | 7.21 | 1.8 | — | 2.41 | 5.8 | No | 2.17 | 4.8 | No |
| Type A | | | | | | | | | | | | |
| (28-B5) | | | | | | | | | | | | |
| Amorphous | 0.013 | 5.9 | No | 5.32 | 5.5 | No | 0.65 | 6.6 | No | 0.008 | 5.1 | No |
| free base | | | | | | | | | | | | |
| (01-A) | | | | | | | | | | | | |

S: Solubility, mg/mL. FC: Form change.
*Additional 23.2 mg of salt was added because clear solution was observed at 10 mg/mL.
—: XRPD test was not performed because limited amount of solid was obtained after solubility test.

### Example 5: Hygroscopicity of 3-Deuteromitragynine Salt Leads

To investigate the solid form stability as a function of humidity, DVS isotherm plots of 3-DM Fumarate Type A, Glycolate Type A, L-Lactate Type A, Succinate Type A, and Mesylate Type A were collected at 25° C. between 0 and 95% relative humidity (RH). After DVS testing, samples were characterized by XRPD to check for form conversion. The results are summarized in Table 20.

For Fumarate Type A, Glycolate Type A, and L-Lactate Type A, water uptake at 80% RH was 0.41%, 0.098%, and 0.31%, respectively. No form change was observed by XRPD after DVS test for any of these 3 salts.

For Succinate Type A and Mesylate Type A, water uptake at 80% RH was 0.16% and 0.49%, respectively. In these samples, some residual water in initial samples was removed during desorption cycle, which resulted in a negative mass change. No form change was observed by XRPD after DVS test for either of these salts.

TABLE 20
DVS results for 3-DM salts.

| 3-DM Salt(Sample ID) | Water uptake at 80% RH | Form change |
|---|---|---|
| Fumarate Type A | 0.41% | No |
| (810397-28-B1) | | |
| Glycolate Type A | 0.098% | No |
| (810397-28-B2) | | |
| L-Lactate Type A | 0.31% | No |
| (810397-28-B3) | | |
| Succinate Type A | 0.16% | No |
| (810397-28-B4) | | |
| Mesylate Type A | 0.49% | No |
| (810397-28-B5) | | |

### Example 6: Physical and Chemical Stability of 3-Deuteromitragynine Salt Leads

The physical and chemical stability of 3-DM Fumarate Type A, Glycolate Type A, L-Lactate Type A, Succinate Type A, and Mesylate Type A were evaluated under conditions of 25° C./60% RH and 40° C./75% RH for 1 week. Each sample was added into 3-mL glass vials, sealed by parafilm with several holes, and stored under one of the indicated test conditions. After one week, samples were taken for XRPD, KF, and HPLC purity tests. All the resulting characterization data are summarized in Table 21.

For all tested salt leads, no purity decrease (by HPLC) or form change (by XRPD) was observed after storage under either of the two conditions for one week. Impurity summaries for all salt leads as determined by HPLC are shown in Table 22 to Table 26. The peak at RRT=0.90 corresponds to impurity 3-DCR. For Glycolate Type A and L-Lactate Type A, the area percentage of 3-DCR slightly decreased after storage under 25° C./60% RH for one week, while no significant change was observed for the other three salt leads.

TABLE 21
Summary of physicochemical stability results for 3-DM salts.

| | | | 25° C./60% RH/1 week | | | 40° C./75% RH/1 week | | |
|---|---|---|---|---|---|---|---|---|
| | Initial | | | Purity/Initial | | | Purity/Initial | |
| 3-DM Salt | Purity | KF | Form | purity | KF | Form | purity | KF |
| (810397-) | (area %) | (wt %) | change | (%) | (wt %) | change | (%) | (wt %) |
| Fumarate | 96.89 | 0.71 | No | 100.3 | 0.79 | No | 100.3 | 0.31 |
| Type A | | | | | | | | |
| (28-B1) | | | | | | | | |
| Glycolate | 98.69 | 0.43 | No | 100.4 | 0.28 | No | 100.4 | 0.26 |
| Type A | | | | | | | | |
| (28-B2) | | | | | | | | |
| L-Lactate | 98.18 | 0.31 | No | 100.7 | 0.64 | No | 100.1 | 0.67 |
| Type A | | | | | | | | |
| (28-B3) | | | | | | | | |
| Succinate | 97.92 | 0.41 | No | 100.0 | 0.39 | No | 99.9 | 0.35 |
| Type A | | | | | | | | |
| (28-B4) | | | | | | | | |
| Mesylate | 96.48 | 0.50 | No | 100.3 | 0.54 | No | 100.3 | 0.54 |
| Type A | | | | | | | | |
| (28-B5) | | | | | | | | |

TABLE 22
Impurity summary of 3-DM Fumarate Type A.

Area (%)

| | | | 25° C./ | 40° C./ |
|---|---|---|---|---|
| #Peak | RRT | Initial | 60% RH | 75% RH |
| 1 | 0.68 | 0.14 | 0.08 | 0.09 |
| 2 | 0.70 | 0.26 | 0.18 | 0.20 |
| 3 | 0.90 | 2.51 | 2.42 | 2.45 |
| 4 | 0.93 | 0.15 | 0.10 | 0.11 |
| 5 | 0.94 | 0.05 | <0.05 | <0.05 |
| 6 | 1.00 | 96.89 | 97.21 | 97.16 |

TABLE 23
Impurity summary of 3-DM Glycolate Type A.

Area (%)

| | | | 25° C./ | 40° C./ |
|---|---|---|---|---|
| #Peak | RRT | Initial | 60% RH | 75% RH |
| 1 | 0.68 | 0.06 | <0.04 | <0.04 |
| 2 | 0.70 | 0.09 | 0.04 | 0.04 |
| 3 | 0.90 | 1.10 | 0.74 | 0.77 |
| 4 | 0.93 | 0.06 | 0.12 | 0.05 |
| 5 | 1.00 | 98.69 | 99.10 | 99.13 |

TABLE 24
Impurity summary of 3-DM L-Lactate Type A.

Area (%)

| | | | 25° C./ | 40° C./ |
|---|---|---|---|---|
| #Peak | RRT | Initial | 60% RH | 75% RH |
| 1 | 0.68 | 0.10 | <0.05 | 0.07 |
| 2 | 0.70 | 0.15 | 0.05 | 0.11 |
| 3 | 0.90 | 1.50 | 0.96 | 1.46 |
| 4 | 0.93 | 0.07 | 0.12 | 0.07 |
| 5 | 1.00 | 98.18 | 98.87 | 98.28 |

TABLE 25
Impurity summary of 3-DM Succinate Type A.

Area (%)

| | | | 25° C./ | 40° C./ |
|---|---|---|---|---|
| #Peak | RRT | Initial | 60% RH | 75% RH |
| 1 | 0.68 | 0.05 | 0.04 | 0.05 |
| 2 | 0.70 | 0.09 | 0.08 | 0.11 |
| 3 | 0.90 | 1.87 | 1.86 | 1.96 |
| 4 | 0.93 | 0.07 | 0.07 | 0.08 |
| 5 | 1.00 | 97.92 | 97.96 | 97.80 |

TABLE 26
Impurity summary of 3-DM Mesylate Type A.

Area (%)

| | | | 25° C./ | 40° C./ |
|---|---|---|---|---|
| #Peak | RRT | Initial | 60% RH | 75% RH |
| 1 | 0.68 | 0.23 | 0.16 | 0.15 |
| 2 | 0.70 | 0.36 | 0.27 | 0.27 |
| 3 | 0.90 | 2.65 | 2.61 | 2.62 |
| 4 | 0.93 | 0.17 | 0.11 | 0.11 |
| 5 | 0.94 | 0.06 | <0.05 | <0.05 |
| 6 | 1.00 | 96.48 | 96.79 | 96.79 |
| 7 | 1.10 | <0.05 | 0.06 | 0.07 |

### Example 7: Scale-up of 3-Deuteromitragynine Glycolate Type A

Based on evaluation results of salt leads, 3-DM Glycolate Type A was selected as a favorable solid form. To select a suitable solvent for scale up of Glycolate Type A with higher yield, approximate solubility of 3-DM Glycolate Type A and 3-DM free base was measured, with the results summarized in Table 27. Based on these findings, isopropyl alcohol (IPA) was selected as the solvent of choice since 3-DM free base showed high solubility and 3-DM Glycolate Type A showed low solubility in IPA. A total of three batches of Glycolate Type A were prepared using solution crystallization methods. In the first batch (100 mg scale), pure IPA was used as solvent. The purity of the product was 97.56% (area percentage), which was lower than the sample obtained from screening. Since a sample obtained from IPA/H₂O during screening showed higher purity, a small amount of water was added to the crystallization solvent in the latter two bathes. Characterization results for the 3 batches are summarized in Table 28. Detailed preparation procedures for the 3 scaled-up batches can be found below.

TABLE 27
Approximate solubility of 3-DM Glycolate
Type A and 3-DM free base.

| 3-DM Glycolate Type A | | 3-DM Free Base | |
|---|---|---|---|
| (810397-36-B2) | | (810397-01-A) | |
| | Solubility | Solvent | Solubility |
| Solvent (v/v) | (mg/mL) | (v/v) | (mg/mL) |
| MeOH | S > 36.0 | MeOH | S > 46.0 |
| EtOH | 6.7 < S < 10.0 | EtOH | S > 38.0 |
| IPA | 1.1 < S < 2.1 | IPA | S > 36.0 |
| Acetone | 6.7 < S < 10.0 | Acetone | S > 38.0 |
| EtOAc | 1.1 < S < 2.1 | EtOAc | S > 40.0 |
| Acetone/H₂O (2:1) | S > 38.0 | — | — |
| Acetone/n-heptane | 1.2 < S < 2.4 | Acetone/n- | S > 42.0 |
| (1:1) | | heptane (1:1) | |
| EtOH/n-heptane | 2.0 < S < 4.0 | — | — |
| (1:1) | | | |

TABLE 28
Summary data for scaled up batches of 3-DM Glycolate Type A.

| Sample ID | 810397-56-B | 810397-62-B | 810397-64-B |
|---|---|---|---|
| Scale | 100 mg | 1 g | 5 g |
| Final solvent system | IPA | IPA/H₂O | IPA/H₂O |
| | | (19:1, v/v) | (19:1, v/v) |
| Yield (%) | 88.3 (105.4 mg) | 78.8 (945.4 mg) | 83.7 (4989.7 mg) |
| XRPD | Glycolate Type A | Glycolate Type A | Glycolate Type A |
| TGA weight loss (%) | 2.2 (to 150° C.) | 1.7 (to 150° C.) | 1.1 (to 150° C.) |
| DSC endotherm | 220.2 | 223.3 | 222.1 |
| (° C., onset) | | | |
| Molar ratio | 1:1 | 1:1 | 1:1 |
| (acid/base) | | | |
| Morphology | Needle-like | Needle-like | Rod-like |
| Purity 3-DM | 97.56 | 98.89 | 98.81 |
| (area %) 3-DCR | 1.96 | 0.95 | 1.02 |

### Preparation of 3-DM Glycolate Type A at 100-mg Scale

1) Weighed 28.1 mg of glycolic acid into a 3-mL glass vial. Added 724 μL IPA to dissolve solids for acid solution.
2) Weighed 99.9 mg of 3-DM free base into a 5-mL glass vial. Added 1.5 mL IPA to dissolve solids for free base solution.
3) Added 0.5 mL acid solution dropwise into free base solution while stirring. Precipitate was observed.
4) Magnetically stirred the suspension at RT for ˜5 min.
5) Isolated solids by centrifugation (10,000 rpm, 2 min). Dried solids at RT under vacuum for ˜17 hrs.
6) A total of 105.4 mg of white solids (needle-like crystals by PLM) were obtained (Yield: 88.3%).

### Preparation of 3-DM Glycolate Type A at 1-g Scale

1) Weighed 197.8 mg of glycolic acid into a 20-mL glass vial. Added 5 mL IPA/H₂O (4:1, v/v) to dissolve solids for acid solution.
2) Weighed 1002.0 mg of 3-DM free base (810397-01-A) into a 50-mL glass vial. Added 15 mL IPA to dissolve solids for free base solution.
3) Added all acid solution dropwise into free base solution while stirring. Thick suspension was observed.
4) Magnetically stirred the suspension at RT for ˜17 hrs.
5) Isolated solids by vacuum filtration. Dried solids at 50° C. under vacuum for ˜3 hrs.
6) A total of 945.4 mg of white solids (needle-like crystals by PLM) were obtained (Yield: 78.8%).

### Preparation of 3-DM Glycolate Type A at 5-g Scale

1) Weighed 961.5 mg of glycolic acid into a 50-mL glass vial. Added 22.5 mL IPA/H₂O (4:1, v/v) to dissolve solids for acid solution.
2) Weighed 5002.5 mg of 3-DM free base (810397-01-A) into a 100-mL glass vial. Added 67.5 mL IPA to dissolve solids for free base solution.
3) Overhead stirred (250 rpm) the free base solution at RT. Added all acid solution into free base solution over 2 hrs.
4) Overhead stirred the obtained suspension at RT for ˜13 hrs.
5) Isolated solids by vacuum filtration. Dried solids at 50° C. under vacuum for ˜5 hrs.
6) A total of 4989.7 mg of white solids (rod-like crystals by PLM) were obtained (Yield: 83.7%).

### Example 8: Solvent Solubility of 3-Deuteromitragynine Glycolate Type A

The approximate solubility of 3-DM Glycolate Type A was measured in 28 solvent systems at RT. Approximately 2 mg samples were added into 3-mL glass vials. The solvents in Table 29 were then added stepwise (50/50/200/700 μL) into the vials until the solids were dissolved visually or a total volume of 1 mL was reached. The solubility results summarized in Table 29 were used to guide the solvent selection for polymorph screening experiments.

TABLE 29
Approximate solubility of 3-DM Glycolate Type A at RT.

| | Solubility | Solvent | Solubility |
|---|---|---|---|
| Solvent | (mg/mL) | (v/v) | (mg/mL) |
| MeOH | S > 36.0 | 1,4-Dioxane | 10.0 < S < 20.0 |
| EtOH | 6.7 < S < 10.0 | ACN | 2.2 < S < 3.7 |
| IPA | 1.1 < S < 2.1 | DCM | 2.2 < S < 3.7 |
| Acetone | 6.7 < S < 10.0 | CHCl₃ | 1.1 < S < 2.2 |
| MIBK | 2.1 < S < 3.5 | n-Heptane | S < 1.1 |
| EtOAc | 1.1 < S < 2.1 | Toluene | S < 1.0 |
| IPAc | 0.9 < S < 1.8 | DMSO | S > 44.0 |
| MTBE | S < 1.0 | DMF | S > 40.0 |
| THF | 19.0 < S < 38.0 | NMP | S > 42.0 |
| 2-MeTHF | 6.7 < S < 10.0 | H₂O | S = 5.3* |

*Solubility was measured in water at 37° C. after rolling for 24 hrs.

### Example 9: Polymorph Screening of 3-DM Glycolate

Polymorph screening experiments were performed under 100 conditions using different solid transition or solution crystallization methods. Methods and results are summarized in Table 30, and further described in detail in the subsections below. A total of six crystal forms of 3-DM Glycolate were obtained as different polymorphic forms.

TABLE 30
Summary of methods and results from polymorph
screening of 3-DM Glycolate.

| | No. of | |
|---|---|---|
| Method | Experiments | Results |
| Anti-solvent addition | 12 | Glycolate Type A, B, C, A + B, |
| | | amorphous |
| Solid vapor diffusion | 12 | Glycolate Type A, C |
| Slurry at RT | 25 | Glycolate Type A |
| Slurry at 50° C. | 16 | Glycolate Type A, C |
| Slow evaporation | 7 | Glycolate Type A, C, E |
| Slow cooling | 6 | Glycolate Type A, B, C |
| Liquid-vapor diffusion | 12 | Glycolate Type A, B, D, A + C |
| Polymer induced | 6 | Glycolate Type A, C, |
| crystallization | | amorphous |
| Grind | 4 | Glycolate Type A |
| Total | 100 | Glycolate Type A-E, A + B, |
| | | A + C, amorphous |

The XRPD spectra of all polymorphic forms are shown in FIG. 30 and the 2-theta peaks are shown below in Table 31.

TABLE 31
XRPD copper radiation results for 3-DM Glycolate Types.

| 3-DM | Pos. | Height | FWHM | d-spacing | Rel. Int. |
|---|---|---|---|---|---|
| Glycolate Salt | [°2θ] | [cts] | Left [°2θ] | [Å] | [%] |
| Type A | 7.06 | 2109.45 | 0.0768 | 12.53 | 61.58 |
| | 10.11 | 3425.57 | 0.1535 | 8.75 | 100.00 |
| | 11.24 | 2400.98 | 0.1023 | 7.87 | 70.09 |
| | 13.21 | 492.74 | 0.1023 | 6.70 | 14.38 |
| | 14.09 | 571.47 | 0.1023 | 6.29 | 16.68 |
| | 15.05 | 86.69 | 0.1535 | 5.89 | 2.53 |
| | 15.96 | 1576.20 | 0.1791 | 5.55 | 46.01 |
| | 18.01 | 1283.35 | 0.1535 | 4.93 | 37.46 |
| | 18.53 | 290.83 | 0.1023 | 4.79 | 8.49 |
| | 19.21 | 399.41 | 0.0768 | 4.62 | 11.66 |
| | 19.49 | 1013.08 | 0.1535 | 4.55 | 29.57 |
| | 19.70 | 605.75 | 0.1023 | 4.51 | 17.68 |
| | 20.34 | 782.93 | 0.1023 | 4.37 | 22.86 |
| | 20.88 | 1902.18 | 0.1791 | 4.25 | 55.53 |
| | 22.57 | 1536.47 | 0.2558 | 3.94 | 44.85 |
| | 23.66 | 149.95 | 0.2047 | 3.76 | 4.38 |
| | 24.00 | 227.34 | 0.1023 | 3.71 | 6.64 |
| | 25.15 | 1552.35 | 0.1535 | 3.54 | 45.32 |
| | 27.62 | 546.00 | 0.1535 | 3.23 | 15.94 |
| | 29.53 | 93.41 | 0.2047 | 3.02 | 2.73 |
| | 30.14 | 254.98 | 0.0768 | 2.96 | 7.44 |
| | 31.61 | 252.54 | 0.1535 | 2.83 | 7.37 |
| | 34.14 | 65.70 | 0.3070 | 2.63 | 1.92 |
| Type B | 5.29 | 1520.48 | 0.0768 | 16.70 | 16.07 |
| | 5.68 | 9462.35 | 0.1279 | 15.57 | 100.00 |
| | 6.78 | 377.43 | 0.1023 | 13.04 | 3.99 |
| | 7.45 | 639.06 | 0.1535 | 11.87 | 6.75 |
| | 9.01 | 67.39 | 0.4093 | 9.81 | 0.71 |
| | 10.78 | 224.66 | 0.1023 | 8.21 | 2.37 |
| | 13.65 | 303.10 | 0.1279 | 6.49 | 3.20 |
| | 14.71 | 99.83 | 0.3070 | 6.02 | 1.05 |
| | 17.35 | 171.94 | 0.1535 | 5.11 | 1.82 |
| | 19.86 | 353.67 | 0.2047 | 4.47 | 3.74 |
| | 21.22 | 161.91 | 0.5117 | 4.19 | 1.71 |
| | 22.72 | 405.05 | 0.1535 | 3.91 | 4.28 |
| | 24.05 | 135.60 | 0.3070 | 3.70 | 1.43 |
| | 25.35 | 73.51 | 0.6140 | 3.51 | 0.78 |
| | 27.43 | 198.64 | 0.2558 | 3.25 | 2.10 |
| Type C | 6.03 | 19774.56 | 0.1023 | 14.66 | 100.00 |
| | 7.43 | 2767.27 | 0.1279 | 11.90 | 13.99 |
| | 10.57 | 212.10 | 0.1023 | 8.37 | 1.07 |
| | 10.99 | 381.18 | 0.1023 | 8.05 | 1.93 |
| | 11.39 | 351.48 | 0.1023 | 7.77 | 1.78 |
| | 12.81 | 162.61 | 0.1023 | 6.91 | 0.82 |
| | 13.65 | 396.34 | 0.1023 | 6.49 | 2.00 |
| | 14.22 | 429.31 | 0.1279 | 6.23 | 2.17 |
| | 15.66 | 124.37 | 0.1535 | 5.66 | 0.63 |
| | 16.30 | 524.59 | 0.1279 | 5.44 | 2.65 |
| | 17.61 | 185.19 | 0.3582 | 5.04 | 0.94 |
| | 18.07 | 541.80 | 0.1791 | 4.91 | 2.74 |
| | 18.84 | 460.35 | 0.1023 | 4.71 | 2.33 |
| | 20.08 | 786.88 | 0.1279 | 4.42 | 3.98 |
| | 21.15 | 258.68 | 0.3070 | 4.20 | 1.31 |
| | 21.54 | 366.62 | 0.0768 | 4.13 | 1.85 |
| | 22.57 | 161.98 | 0.4093 | 3.94 | 0.82 |
| | 22.87 | 370.26 | 0.1279 | 3.89 | 1.87 |
| | 24.16 | 1612.07 | 0.1535 | 3.68 | 8.15 |
| | 25.14 | 157.20 | 0.3070 | 3.54 | 0.79 |
| | 26.22 | 441.23 | 0.1535 | 3.40 | 2.23 |
| | 27.55 | 465.02 | 0.1535 | 3.24 | 2.35 |
| | 28.60 | 111.96 | 0.2047 | 3.12 | 0.57 |
| | 29.38 | 79.84 | 0.2047 | 3.04 | 0.40 |
| | 31.63 | 79.96 | 0.3070 | 2.83 | 0.40 |
| | 33.74 | 84.29 | 0.1535 | 2.66 | 0.43 |
| | 35.22 | 56.07 | 0.3070 | 2.55 | 0.28 |
| | 38.31 | 44.82 | 0.3070 | 2.35 | 0.23 |
| Type D | 5.33 | 2619.84 | 0.1023 | 16.57 | 100.00 |
| | 6.82 | 1316.79 | 0.1023 | 12.97 | 50.26 |
| | 9.04 | 588.62 | 0.1791 | 9.78 | 22.47 |
| | 10.09 | 189.02 | 0.1023 | 8.77 | 7.21 |
| | 11.02 | 486.13 | 0.1023 | 8.03 | 18.56 |
| | 11.33 | 328.81 | 0.1023 | 7.81 | 12.55 |
| | 13.53 | 616.04 | 0.0768 | 6.54 | 23.51 |
| | 13.85 | 369.18 | 0.1023 | 6.40 | 14.09 |
| | 16.48 | 149.73 | 0.1535 | 5.38 | 5.72 |
| | 16.99 | 249.21 | 0.0768 | 5.22 | 9.51 |
| | 17.28 | 386.64 | 0.1279 | 5.13 | 14.76 |
| | 17.98 | 138.74 | 0.3070 | 4.93 | 5.30 |
| | 19.50 | 390.10 | 0.1023 | 4.55 | 14.89 |
| | 20.08 | 385.54 | 0.1023 | 4.42 | 14.72 |
| | 21.28 | 484.13 | 0.1279 | 4.18 | 18.48 |
| | 22.88 | 193.33 | 0.1535 | 3.89 | 7.38 |
| | 23.71 | 353.50 | 0.1535 | 3.75 | 13.49 |
| | 25.50 | 97.46 | 0.1535 | 3.49 | 3.72 |
| | 27.27 | 145.81 | 0.1535 | 3.27 | 5.57 |
| Type E | 5.10 | 166.04 | 0.1535 | 17.32 | 52.30 |
| | 7.81 | 317.45 | 0.1791 | 11.32 | 100.00 |
| | 8.82 | 179.65 | 0.2047 | 10.03 | 56.59 |
| | 10.97 | 151.26 | 0.2047 | 8.06 | 47.65 |
| | 12.02 | 138.74 | 0.1535 | 7.36 | 43.70 |
| | 15.14 | 70.19 | 0.5117 | 5.85 | 22.11 |
| | 16.79 | 111.00 | 0.1535 | 5.28 | 34.97 |
| | 18.40 | 66.56 | 0.4093 | 4.82 | 20.97 |
| | 19.08 | 97.22 | 0.3070 | 4.65 | 30.63 |
| | 20.91 | 76.12 | 0.4093 | 4.25 | 23.98 |
| | 22.25 | 69.11 | 0.6140 | 4.00 | 21.77 |
| Type F | 5.86 | 9925.04 | 0.1004 | 15.09 | 100.00 |
| | 6.39 | 3049.85 | 0.0836 | 13.84 | 30.73 |
| | 7.21 | 1668.11 | 0.0836 | 12.26 | 16.81 |
| | 10.26 | 342.75 | 0.1338 | 8.62 | 3.45 |
| | 10.83 | 1177.50 | 0.0669 | 8.17 | 11.86 |
| | 11.22 | 1052.61 | 0.0836 | 7.88 | 10.61 |
| | 12.30 | 344.98 | 0.2007 | 7.20 | 3.48 |
| | 13.41 | 1274.77 | 0.0836 | 6.60 | 12.84 |
| | 14.05 | 1246.30 | 0.0836 | 6.30 | 12.56 |
| | 16.13 | 1222.17 | 0.1004 | 5.49 | 12.31 |
| | 16.84 | 803.92 | 0.2007 | 5.27 | 8.10 |
| | 17.23 | 750.76 | 0.2007 | 5.15 | 7.56 |
| | 17.76 | 1065.21 | 0.1338 | 4.99 | 10.73 |
| | 18.42 | 1503.52 | 0.2007 | 4.82 | 15.15 |
| | 19.81 | 1904.97 | 0.0669 | 4.48 | 19.19 |
| | 20.58 | 859.81 | 0.1004 | 4.32 | 8.66 |
| | 21.36 | 1393.30 | 0.0836 | 4.16 | 14.04 |
| | 22.07 | 1013.11 | 0.1171 | 4.03 | 10.21 |
| | 23.68 | 1580.69 | 0.0669 | 3.76 | 15.93 |
| | 23.87 | 1542.75 | 0.1004 | 3.73 | 15.54 |
| | 24.38 | 999.19 | 0.2342 | 3.65 | 10.07 |
| | 25.99 | 634.61 | 0.2676 | 3.43 | 6.39 |
| | 26.44 | 649.77 | 0.1338 | 3.37 | 6.55 |
| | 27.19 | 596.23 | 0.2342 | 3.28 | 6.01 |

All crystal forms that could be obtained at ambient conditions were further characterized by TGA, DSC, and ¹H NMR.

### 3-DM Glycolate Type A

3-DM Glycolate Type A was prepared via solution crystallization in IPA/H₂O solvent system. Detailed preparation procedures and characterization results can be found in Examples 2-8 above. Glycolate Type A was speculated to be an anhydrate due to the small TGA weight loss and neat DSC signal.

### 3-DM Glycolate Type B

3-DM Glycolate Type B was obtained via vapor diffusion of a THF solution of 3-DM Glycolate Type A in cyclohexane atmosphere. The XRPD pattern is shown in FIG. 30, which is consistent with that of Glycolate Type B obtained during salt screening. TGA showed a weight loss of 3.6% up to 120° C. and a stepwise weight loss of 8.2% between 120° C. and 160° C. DSC showed a weak endotherm at 146.9° C. and a sharp endotherm at 222.7° C. (peak). ¹H NMR indicated that the molar ratio of residual solvent (cyclohexane) to 3-DM was 0.4:1 (the corresponding TGA weight loss was 6.3%). The molar ratio of acid to free base was 1:1. XRPD showed that no form change was observed after heating 3-DM Glycolate Type B to 120° C. and cooling to RT. For the sample after heating to 120° C., TGA, DSC and ¹H NMR characterization were performed again. TGA showed a weight loss of 3.5% up to 120° C. and a stepwise weight loss of 7.6% between 120° C. and 170° C. DSC showed a weak endotherm at 146.3° C. and a sharp endotherm at 214.7° C. (peak). ¹H NMR showed that the molar ratio of residual solvent (cyclohexane) to 3-DM had decreased to 0.2:1 (the corresponding TGA weight loss was 3.9%). As no form change occurred after removal of much of the solvent, the residual solvent might be surface-adsorbed. Based on the results, 3-DM Glycolate Type B is likely to be a hydrate or anhydrate. After heating to 160° C. and cooling to RT, 3-DM Glycolate Type A was obtained.

### 3-DM Glycolate Type C

3-DM Glycolate Type C was obtained via vapor diffusion of 3-DM Glycolate Type A in CHCl₃ atmosphere. The XRPD pattern is shown in FIG. 30. TGA showed a weight loss of 5.9% up to 150° C. DSC showed two endotherms at 61.2° C. and 222.0° C. (peak), and an exotherm at 140.8° C. (peak). ¹H NMR showed that the molar ratio of acid to free base was 1:1, while the solvent CHCl₃ was not detected. After heating 3-DM Glycolate Type C to 100° C. and cooling to RT, no form change was observed. 3-DM Glycolate Type A was obtained after heating to 150° C. and cooling to RT.

Another batch of 3-DM Glycolate Type C was obtained via slurry of 3-DM Glycolate Type A in CHCl₃ at 50° C. XRPD was consistent with the material obtained by vapor diffusion. TGA showed a weight loss of 2.9% up to 150° C. DSC showed two endotherms at 57.3° C. and 219.7° C. (peak), and an exotherm at 134.7° C. (peak). 1H NMR indicated that the molar ratio of acid to free base was 1:1, while the solvent CHCl₃ was not detected. After N₂ sweeping 3-DM Glycolate Type C for 20 min at 30° C., a new form was observed, which was classified as 3-DM Glycolate Type F. After exposure of Type F to ambient conditions for 1 hr, Glycolate Type C was re-obtained. Based on these results, glycolate Type C was speculated to be a hydrate as it could be re-obtained after absorbing water in air during exposure to ambient conditions.

### 3-DM Glycolate Type D

3-DM Glycolate Type D was obtained via vapor diffusion of 1,4-dioxane solution of 3-DM Glycolate Type A in an MTBE atmosphere. The XRPD pattern is shown in FIG. 30. TGA showed a weight loss of 2.6% up to 100° C. DSC showed two endotherms at 63.0° C. and 210.3° C. (peak), and an exotherm at 123.2° C. (peak). ¹H NMR showed that the molar ratio of acid to free base was 1:1, while the solvents 1,4-dioxane and MTBE were not detected. Variable temperature XRPD was performed for further characterization of 3-DM Glycolate Type D. No form change was observed after heating 3-DM Glycolate Type D to 90° C. with N₂ protection. As a result, 3-DM Glycolate Type D was speculated to be an anhydrate. However, after heating Glycolate Type D (810397-74-A8) to 150° C. and cooling to RT, Glycolate Type A was observed by XRPD.

### 3-DM Glycolate Type E

3-DM Glycolate Type E was obtained via slow evaporation of an EtOH solution of 3-DM Glycolate Type A at RT. The XRPD pattern is shown in FIG. 30. Form conversion to Glycolate Type A was observed after drying Glycolate Type E at RT.

### 3-DM Glycolate Type F

3-DM Glycolate Type F was obtained after N₂ sweeping 3-DM Glycolate Type C at 30° C. for 20 min. The XRPD pattern is shown in FIG. 30. After exposure of Glycolate Type F to ambient conditions for 30 min, Glycolate Type C was re-obtained.

### Anti-Solvent Addition

For each experiment, approximately 15 mg of 3-DM Glycolate Type A was added into a 20-mL glass vial and dissolved in 0.1-1.6 mL of corresponding solvent to obtain a clear solution. The solution was magnetically stirred with addition of anti-solvent until precipitates appeared or the total volume of anti-solvent reached 10 mL. The obtained solids were isolated for XRPD analysis. If solids were not obtained, slurry at 5° C. and/or evaporation at RT was performed. The results (Table 32) showed that Glycolate Type A, B, C, A+B, and amorphous samples were generated.

TABLE 32
Summary of anti-solvent addition experiments
for 3-DM Glycolate Type A.

| Experiment ID | Solvent (v/v) | Anti-solvent | Result |
|---|---|---|---|
| 810397-68-A1 | MeOH | MTBE | Glycolate Type B |
| 810397-68-A2 | | Toluene | Glycolate Type A |
| 810397-68-A3 | | CHCl₃ | Glycolate Type C |
| 810397-68-A4 | | H₂O | Amorphous |
| 810397-68-A5 | THF | n-Heptane | Glycolate Type C |
| 810397-68-A6 | | Toluene | Glycolate Type C |
| 810397-68-A7 | | MTBE | Glycolate Type A + B |
| 810397-68-A8 | | Cyclohexane | Glycolate Type C |
| 810397-68-A9 | DMSO | MTBE | Glycolate Type A |
| 810397-68-A10 | | Toluene | Glycolate Type A |
| 810397-68-A11 | | IPAc | Glycolate Type A + B |
| 810397-68-A12 | | H₂O | Glycolate Type A |

### Solid-Vapor Diffusion

Solid vapor diffusion experiments were conducted using 12 different solvents. For each experiment, approximately 15-mg of 3-DM Glycolate Type A was weighed into a 3-mL vial, which was placed into a 20-mL vial with 4 mL of volatile solvent. The 20-mL vial was sealed with a cap and kept at RT for 7 days allowing solvent vapor to interact with the sample. The solids were tested by XRPD and the results, summarized in Table 33, showed that Glycolate Type A and C were obtained.

TABLE 33
Summary of solid-vapor diffusion experiments
for 3-DM Glycolate Type A.

| Experiment ID | Solvent | Result |
|---|---|---|
| 810397-69-A1 | H₂O | Glycolate Type A |
| 810397-69-A2 | DCM | Glycolate Type A |
| 810397-69-A3 | EtOH | Glycolate Type A |
| 810397-69-A4 | MeOH | Glycolate Type A |
| 810397-69-A5 | ACN | Glycolate Type A |
| 810397-69-A6 | THF | Glycolate Type A |
| 810397-69-A7 | CHCl₃ | Glycolate Type C |
| 810397-69-A8 | Acetone | Glycolate Type A |
| 810397-69-A9 | DMSO | Glycolate Type A |
| 810397-69-A10 | EtOAc | Glycolate Type A |
| 810397-69-A11 | 1,4-Dioxane | Glycolate Type A |
| 810397-69-A12 | IPA | Glycolate Type A |

### Slurry at RT

For each experiment, approximately 15 mg of 3-DM Glycolate Type A was suspended in 0.5 mL of corresponding solvent in an HPLC vial. After the suspension was stirred magnetically (˜1000 rpm) for 4 days at RT, the remaining solids were isolated by centrifugation for XRPD analysis. The results, summarized in Table 34, indicated that only Glycolate Type A was generated.

TABLE 34
Summary of slurry conversion experiments
at RT for 3-DM Glycolate Type A.

| Sample ID | Solvent (v/v) | Crystal Form |
|---|---|---|
| 810397-70-A1 | EtOH | Glycolate Type A |
| 810397-70-A2 | IPA | Glycolate Type A |
| 810397-70-A3 | Acetone | Glycolate Type A |
| 810397-70-A4 | MIBK | Glycolate Type A |
| 810397-70-A5 | EtOAc | Glycolate Type A |
| 810397-70-A6 | IPAc | Glycolate Type A |
| 810397-70-A7 | MTBE | Glycolate Type A |
| 810397-70-A8 | 2-MeTHF | Glycolate Type A |
| 810397-70-A9 | ACN | Glycolate Type A |
| 810397-70-A10 | DCM | Glycolate Type A |
| 810397-70-A11 | Toluene | Glycolate Type A |
| 810397-70-A12 | H₂O | Glycolate Type A |
| 810397-70-A13 | MeOH/Toluene (1:4) | Glycolate Type A |
| 810397-70-A14 | Acetone/EtOAc (1:1) | Glycolate Type A |
| 810397-70-A15 | THF/Toluene (1:1) | Glycolate Type A |
| 810397-70-A16 | EtOH/MEK (1:1) | Glycolate Type A |
| 810397-70-A17 | DMSO/H₂O (1:9) | Glycolate Type A |
| 810397-70-A18 | CPME/n-Heptane (1:1) | Glycolate Type A |
| 810397-70-A19 | ACN/IPAc (1:1) | Glycolate Type A |
| 810397-70-A20 | 1,4-Dioxane/CHCl₃ (1:1) | Glycolate Type A |
| 810397-70-A21 | MIBK/MTBE (1:1) | Glycolate Type A |
| 810397-70-A22 | IPA/H₂O (aw~0.2) | Glycolate Type A |
| 810397-70-A23 | IPA/H₂O (aw~0.4) | Glycolate Type A |
| 810397-70-A24 | IPA/H₂O (aw~0.6) | Glycolate Type A |
| 810397-70-A25 | IPA/H₂O (aw~0.8) | Glycolate Type A |

### Slurry at 50° C.

For each experiment, approximately 15 mg of 3-DM Glycolate Type A was suspended in 0.5 mL of corresponding solvent in an HPLC vial. After the suspension was magnetically stirred (˜1000 rpm) for 3 days at 50° C., the remaining solids were isolated by centrifugation for XRPD analysis. The results, summarized in Table 35, indicated that Glycolate Type A and C were generated.

TABLE 35
Summary of slurry conversion experiments
at 50° C. for 3-DM Glycolate Type A.

| Experiment ID | Solvent (v/v) | Result |
|---|---|---|
| 810397-71-A1 | IPA | Glycolate Type A |
| 810397-71-A2 | MIBK | Glycolate Type A |
| 810397-71-A3 | EtOAc | Glycolate Type A |
| 810397-71-A4 | MTBE | Glycolate Type A |
| 810397-71-A5 | ACN | Glycolate Type A |
| 810397-71-A6 | CHCl₃ | Glycolate Type C |
| 810397-71-A7 | Toluene | Glycolate Type A |
| 810397-71-A8 | H₂O | Glycolate Type A |
| 810397-71-A9 | EtOH/n-Heptane (1:1) | Glycolate Type A |
| 810397-71-A10 | Acetone/Toluene (1:1) | Glycolate Type A |
| 810397-71-A11 | 1,4-Dioxane/H₂O (1:1) | Glycolate Type A |
| 810397-71-A12 | IPA/IPAc (1:1) | Glycolate Type A |
| 810397-71-A13 | ACN/2-MeTHF (1:1) | Glycolate Type A |
| 810397-71-A14 | MIBK/MTBE (1:1) | Glycolate Type A |
| 810397-71-A15 | THF/n-Heptane (1:1) | Glycolate Type A |
| 810397-71-A16 | NMP/H₂O (1:4) | Glycolate Type A |

### Slow Evaporation

Slow evaporation experiments were performed under seven conditions. For each experiment, approximately 15 mg of 3-DM Glycolate Type A was dissolved in 0.4-2.0 mL of corresponding solvent in a 4-mL glass vial and filtered using a 0.45 μm PTFE membrane. The visually clear solution was subjected to evaporation at RT in a vial sealed by a transparent PE-Plug (poke 4 small holes). The solids were isolated for XRPD analysis and the results, summarized in Table 36, indicated that Glycolate Type A, C, and E were obtained.

TABLE 36
Summary of slow evaporation experiments
for 3-DM Glycolate Type A.

| Experiment ID | Solvent | Result |
|---|---|---|
| 810397-72-A1 | MeOH | Glycolate Type A |
| 810397-72-A2 | EtOH | Glycolate Type E |
| 810397-72-A3 | Acetone | Glycolate Type A |
| 810397-72-A4 | EtOAc | Glycolate Type A |
| 810397-72-A5 | THF | Glycolate Type C |
| 810397-72-A6 | DCM | Glycolate Type C |
| 810397-72-A7 | ACN | Glycolate Type A |

### Slow Cooling

For each experiment, approximately 15 mg of 3-DM Glycolate Type A was suspended in 1.0-2.0 mL of corresponding solvent in a 3-mL glass vial at RT. The suspension was then heated to 50° C., stirred for 1.5 hrs, and filtered into a new vial using a 0.45 μm PTFE membrane. Filtrates were slowly cooled down to 5° C. at a rate of 0.1° C./min. The obtained solids were kept isothermal at 5° C. before isolation for XRPD analysis. The results, summarized in Table 37, indicated that Glycolate Type A, B, and C were generated.

TABLE 37
Summary of slow cooling experiments
for 3-DM Glycolate Type A.

| Experiment ID | Solvent (v/v) | Result |
|---|---|---|
| 810397-73-A1^{#} | EtOH | Glycolate Type A |
| 810397-73-A2* | Acetone | Glycolate Type C |
| 810397-73-A3 | ACN | Glycolate Type A |
| 810397-73-A4* | EtOAc/MEK (1:1) | Glycolate Type C |
| 810397-73-A5^{#} | IPA/H₂O (9:1) | Glycolate Type B |
| 810397-73-A6 | THF/CHCl₃ (1:1) | Glycolate Type C |

*Clear solution was obtained after cooling to 5° C., which was transferred to −20° C. for precipitation.
^{#}Clear solution was obtained after cooling to 5° C. and −20° C., so solution was transferred to evaporate at RT.

### Liquid-Vapor Diffusion

For each experiment, approximately 15 mg of 3-DM Glycolate Type A was dissolved in 0.1-1.6 mL of corresponding solvent in a 3-mL glass vial to obtain a clear solution. The 3-mL vial was then placed into a 20-mL vial with 4 mL of anti-solvent. The 20-mL vial was sealed with a cap and kept at RT for 7 days, allowing organic vapor to interact with the solution. The solids were isolated for XRPD analysis. The results, summarized in Table 38, showed that Glycolate Type A, B, D, and A+C were generated.

TABLE 38
Summary of liquid-vapor diffusion experiments
for 3-DM Glycolate Type A.

| Experiment ID | Solvent | Anti-solvent | Result |
|---|---|---|---|
| 810397-74-A1 | EtOH | n-Hexane | Glycolate Type A |
| 810397-74-A2* | | MTBE | Glycolate Type A + C |
| 810397-74-A3 | | Toluene | Glycolate Type A |
| 810397-74-A4 | THF | n-Hexane | Glycolate Type A |
| 810397-74-A5 | | Cyclohexane | Glycolate Type B |
| 810397-74-A6 | | MTBE | Glycolate Type A |
| 810397-74-A7 | 1,4-Dioxane | n-Heptane | Glycolate Type A + C |
| 810397-74-A8* | | MTBE | Glycolate Type D |
| 810397-74-A9* | | H₂O | Amorphous |
| 810397-74-A10* | NMP | Toluene | Glycolate Type A |
| 810397-74-A11* | | MTBE | Glycolate Type A |
| 810397-74-A12 | | H₂O | Glycolate Type A |

*No solids were obtained after vapor diffusion, so solution was transferred to evaporate at RT.

### Polymer-Induced Crystallization

For each experiment, approximately 15 mg of 3-DM Glycolate Type A was dissolved in 1.0-2.0 mL of corresponding solvent in a 4-mL glass vial and filtered using a 0.45 μm PTFE membrane to a new vial containing ˜2 mg of polymers. The visually clear solution was subjected to evaporation at RT in a vial sealed by a transparent PE-Plug (poke 4 small holes). The solids were isolated for XRPD analysis. The results, summarized in Table 39, indicated that Glycolate Type A, C, and amorphous samples were obtained.

TABLE 39
Summary of polymer-induced crystallization
experiments for 3-DM Glycolate Type A.

| Exp. ID | Solvent (v/v) | Polymer | Result |
|---|---|---|---|
| 810397-75-A1 | EtOH | Polymer | Glycolate Type A |
| 810397-75-A2 | MIBK | mixture A | Glycolate Type A |
| 810397-75-A3 | ACN/H₂O (1:1) | | Glycolate Type |
| | | | C + peaks |
| 810397-75-A4 | MTBE | Polymer | Glycolate Type A |
| 810397-75-A5 | IPAc | mixture B | Glycolate Type A |
| 810397-75-A6 | MeOH/DCM (1:1) | | Amorphous |

Polymer mixture A: polyvinyl pyrrolidone (PVP), polyvinyl alcohol (PVA), polyvinylchloride (PVC), polyvinyl acetate (PVAC), hypromellose (HPMC), methyl cellulose (MC) (mass ratio of 1:1:1:1:1:1)

Polymer mixture B: polycaprolactone (PCL), polyethylene glycol (PEG), poly (methyl methacrylate) (PMMA) sodium alginate (SA), and hydroxyethyl cellulose (HEC) (mass ratio of 1:1:1:1:1).

### Grinding

For each experiment, approximately 15 mg of 3-DM Glycolate Type A was added into an agate mortar and manually ground using a pestle for about 3 min after adding the corresponding solvent. The solids were checked by XRPD and the results, summarized in Table 40, showed that only Glycolate Type A was obtained.

TABLE 40
Summary of grinding experiments for 3-DM Glycolate Type A.

| Exp. ID | Solvent | Result |
|---|---|---|
| 810397-76-A1 | None | Glycolate Type A |
| 810397-76-A2 | IPA | Glycolate Type A |
| 810397-76-A3 | MIBK | Glycolate Type A |
| 810397-76-A4 | EtOAc | Glycolate Type A |

### Example 10: Re-Preparation of 3-DM Glycolate Forms

3-DM Glycolate Type B, Type C, and Type D were re-prepared for slurry competition experiments. The characterization results are summarized in Table 41.

TABLE 41
Characterization results of 3-DM Glycolate Type B/C/D.

| | | | Molar | |
|---|---|---|---|---|
| | TGA weight | DSC | ratio | Purity |
| | loss | endotherm | (acid/ | (area |
| Crystal form | (%) | (° C., peak) | API) | %) |
| Glycolate Type B | 3.1 (to 120° C.) | 139.1, 219.7 | 1:1 | 99.4^{#} |
| (810397-81-A1) | 5.0 (120-160° C.) | | | |
| Glycolate Type C | 8.2 (to 150° C.) | 135.0*, 224.0, | 1:1 | 99.1 |
| (810397-69-A7) | | 226.7 | | |
| Glycolate Type D | 3.5 (to 100° C.) | 129.0, 135.7*, | 1:1 | 93.8 |
| (810397-74-A8) | | 210.9 | | |

*Exotherm peak.
^{#}Glycolate Type B converted to Type A + B after storage at ambient condition before HPLC purity test.

### Re-Preparation of 3-DM Glycolate Type B

3-DM Glycolate Type B was prepared via vapor diffusion of THE solution of 3-DM Glycolate Type A in cyclohexane atmosphere. XRPD was consistent with the material prepared during polymorph screening (Example 9). TGA showed a weight loss of 3.1% up to 120° C. and a stepwise weight loss of 5.0% between 120° C. and 160° C. DSC showed a broad endotherm at 139.1° C. and a sharp endotherm at 219.7° C. (peak). ¹H NMR showed that the molar ratio of residual solvent (cyclohexane) to 3-DM was 0.2:1 (the corresponding TGA weight loss was 3.8%) and that the acid/free base ratio was 1:1. HPLC purity was determined as 99.4% (area).

### Re-Preparation of 3-DM Glycolate Type C

3-DM Glycolate Type C was prepared via a slurry of 3-DM Glycolate Type A in CHCl₃ at 50° C., followed by drying of the solids at RT. XRPD was consistent with the material prepared during polymorph screening (Example 9). TGA showed a weight loss of 8.2% up to 150° C. DSC showed an exotherm at 135.0° C. and two endotherms at 224.0° C. and 226.7° C. (peak). ¹H NMR showed that the molar ratio of residual solvent (CHCl₃) to 3-DM was 0.4:1 (the corresponding TGA weight loss was 9.6%) and that the acid/free base ratio was 1:1. HPLC purity was determined as 99.1% (area).

### Re-Preparation of 3-DM Glycolate Type D

3-DM Glycolate Type D was prepared via adding the anti-solvent MTBE into a 1,4-Dioxane solution of 3-DM Glycolate Type A, followed by evaporation at RT. XRPD was consistent with the material prepared during polymorph screening (Example 9). TGA showed a weight loss of 3.5% up to 100° C. DSC showed two endotherms at 129.0° C. and 210.9° C. (peak) and an exotherm at 135.7° C. (peak). ¹H NMR showed that the molar ratio of residual solvent (1,4-dioxane) to 3-DM was 0.3:1 (the corresponding TGA weight loss was 4.9%) and that the acid/free base ratio was 1:1. HPLC purity was determined as 93.8% (area).

### Example 11: Interconversion Relationships Between 3-DM Glycolate Forms

### Interconversion Relationship Between 3-DM Glycolate Types A/B/D

To investigate the thermodynamic stability relationship between 3-DM Glycolate anhydrates, slurry competition experiments between anhydrates Glycolate Type A and Type D and anhydrate/hydrate Type B were performed at RT and 50° C. in EtOH and MIBK. Detailed procedures were as follows.
1) 3-DM Glycolate Type A was used to saturate the corresponding solvent systems at RT and 50° C. before filtration to obtain a near-saturated solution.
2) Equal amounts (about 6 mg each) of 3-DM Glycolate Type A, Type B, and Type D were added into 1 mL of the saturated solution of Type A at RT or 50° C. to form a suspension.
3) The suspension was magnetically stirred at RT or 50° C. for 2 days and the solids were then isolated for XRPD analysis.

Glycolate Type A was obtained under all conditions, indicating that Glycolate Type A was more thermodynamically stable than Glycolate Type B and Type D in the temperature range of RT to 50° C.

### Interconversion Relationship Between 3-DM Glycolate Types A/B/C

To investigate the interconversion relationship between 3-DM Glycolate Type A, Type B, and Type C, slurry competition experiments were performed in IPA/H₂O with water activity aw=0-1 at RT. Detailed procedures are as follows.
1) 3-DM Glycolate Type A was used to saturate the corresponding solvent systems at RT before filtration to obtain a near-saturated solution.
2) Equal amounts (about 7 mg each) of 3-DM Glycolate Type A, Type B, and Type C were added into 1 mL of the saturated solution of Type A at RT to form a suspension.
3) The suspension was magnetically stirred at RT for 2 days and the solids were then isolated for XRPD analysis.

The results are summarized in Table 42. Glycolate Type A was obtained under all conditions, indicating that Glycolate Type A was more thermodynamically stable than Glycolate Type B and Type C under the tested conditions.

TABLE 42
Results of slurry competition experiment
between 3-DM Glycolate Types A/B/C.

| Experiment ID | Starting form | Solvent (aw) | Temperature | Final form |
|---|---|---|---|---|
| 810397-84-A1 | Glycolate | IPA | RT | Glycolate |
| 810397-84-A2 | Type A, B, | IPA/H₂O | RT | Type A |
| | and C* | (aw~0.2) | | |
| 810397-84-A3 | | IPA/H₂O | RT | |
| | | (aw~0.4) | | |
| 810397-84-A4 | | IPA/H₂O | RT | |
| | | (aw~0.6) | | |
| 810397-84-A5 | | IPA/H₂O | RT | |
| | | (aw~0.8) | | |
| 810397-84-A6 | | H₂O | RT | |

*Glycolate Type B and Type C converted to a mixture of Glycolate Type B + A and Glycolate Type C + A, respectively, before slurry competition.

### Example 12: Solid State Stability Evaluation of 3-DM Glycolate Type A

According to the characterization and interconversion relationship study results, 3-DM Glycolate Type A was selected as the leading form for solid state stability evaluation. 3-DM Glycolate Type A samples were stored under 60° C./closed for 1 day, 25° C./60% RH/open for 2 weeks, and 40° C./75% RH/open for 2 weeks. All the stability samples were characterized by XRPD, HPLC, and Karl Fischer (KF) titration, with the results summarized in Table 43. No form change or significant HPLC purity decrease was observed for 3-DM Glycolate Type A under any condition, indicating good physical and chemical stability.

TABLE 43
Solid state stability evaluation results of 3-DM Glycolate Type A.

| | Initial | | Final/ | | |
|---|---|---|---|---|---|
| | Purity | Purity | Initial | | H₂O |
| | (area | (area | Purity | Form | by KF |
| Condition | %) | %) | (%) | Change | (wt %) |
| 60° C./closed | | 98.81 | 100.1 | No | 0.21 |
| 25° C./60% RH/open | 98.76 | 98.80 | 100.0 | No | 0.40 |
| 40° C./75% RH/open | | 98.75 | 100.0 | No | 0.23 |

### Example 13: Summary of Polymorph Screening of 3-DM Glycolate

Using compound 3-DM Glycolate as the starting material, 100 polymorph screening experiments were performed via anti-solvent addition, solid-vapor diffusion, slurry, slow evaporation, slow cooling, liquid-vapor diffusion, polymer induced crystallization and grinding. A total of six forms of 3-DM Glycolate were obtained during screening, as shown by XRPD: Glycolate Type A (the starting form) and Types B through F. All crystal forms except Glycolate Type E and Type F were further characterized by thermogravimetric analysis (TGA), differential scanning calorimetry (DSC) and proton nuclear magnetic resonance (¹H NMR). A characterization summary for all crystal forms is shown in Table 44. Based on characterization results, Glycolate Type A and D were anhydrates, Glycolate Type C was a hydrate, Glycolate Type B was a hydrate or anhydrate, and Glycolate Type E and F were metastable forms.

Interconversion among Glycolate Types A, B, and D was studied in EtOH and MIBK at room temperature (RT, 23±3° C.) and 50° C. Glycolate Type A was obtained under all conditions, indicating Glycolate Type A was thermodynamically more stable than Glycolate Types B and D from RT to 50° C. Slurry competition experiments among Glycolate Types A, B, and C were conducted in IPA/H₂O (aw=0˜1) at RT. Glycolate Type A was obtained under all conditions. An interconversion diagram of the different forms is displayed below in Scheme 2.

3-DM Glycolate Type A was further characterized in solid state stability studies. Samples were stored under 60° C. for one day, 25° C./60% RH for 2 weeks, and 40° C./75% RH for two weeks. No form change or purity decrease was observed for Glycolate Type A under any condition tested.

TABLE 44
Characterization of 3-DM Glycolate crystal forms.

| | | DSC | Molar | |
|---|---|---|---|---|
| | TGA weight | endotherm | ratio | |
| | loss | (° C., | (acid/ | Speculated |
| Crystal form | (%) | peak) | API) | form |
| Glycolate Type A | 1.1 (to 150° C.) | 222.1 | 1:1 | Anhydrate |
| (810397-64-B) | | | | |
| Glycolate Type B | 3.6 (to 120° C.) | 146.9, | 1:1 | Anhy- |
| (810397-74-A5) | 8.2 | 222.7 | | drate/ |
| | (120~160° C.) | | | hydrate |
| Glycolate Type C | 5.9 (to 150° C.) | 61.2, 140.8*, | 1:1 | Hydrate |
| (810397-69-A7) | | 222.0 | | |
| Glycolate Type D | 2.6 (to 100° C.) | 63.0, 123.2*, | 1:1 | Anhydrate |
| (810397-74-A8) | | 210.3 | | |
| Glycolate Type E | NA | NA | NA | Metastable |
| (810397-72-A2) | | | | |
| Glycolate Type F | — | — | — | Metastable |
| (810397-80-A) | | | | |

*Exotherm peak temperature.
NA: Form converted to Type A after drying, so TGA, DSC, and ¹H NMR characterization were not performed.
—: Type F was only observed under N₂ protection and converted to Type C after exposure to ambient conditions.

A summary of each numbered process step in Scheme 2 above is provided below:
(1) Slurry in EtOH or MIBK at RT and 50° C.
(2) Slurry in IPA/H₂O (aw=0˜1) at RT.
(3) Heating to 160° C. and cooling to 30° C.
(4) Heating to 150° C. and cooling to 30° C.
(5) Solid vapor diffusion in CHCl₃ atmosphere.
(6) Slurry in CHCl₃ at 50° C.
(7) Drying at RT.
(8) N2 sweeping for 20 min 30° C.
(9) Exposure to ambient conditions for 30 min.

### Example 14: Characterization of Starting Materials for 3-DM Glycolate Type A Crystallization Process Development

### 3-DM Free Base

3-Deuteromitragynine (3-DM) free base for use in 3-DM Glycolate Type A crystallization process development experiments (see Examples 18-21) was prepared as previously described above and characterized by XRPD, TGA, mDSC, ¹H NMR, and HPLC. XRPD showed that the material was amorphous. TGA/mDSC revealed a weight loss of 1.6% up to 200° C. and a possible glass transition (Tg) signal at 93.1° C. (middle temperature). ¹H NMR confirmed the identity of the material. HPLC purity was determined as 99.57% (area), with detailed results listed in Table 45. The area percentage of impurity 3-deuterocorynantheidine (3-DCR) was 0.05%.

TABLE 45
HPLC data of 3-DM free base starting material.

| #Peak | RRT | Area (%) |
|---|---|---|
| 1 | 0.69 | 0.08 |
| 2 | 0.70 | 0.18 |
| 3 | 0.73 | 0.12 |
| 4 | 0.90 (3-DCR) | 0.05 |
| 5 | 1.00 (3-DM) | 99.57 |

### 3-DM Glycolate Type A

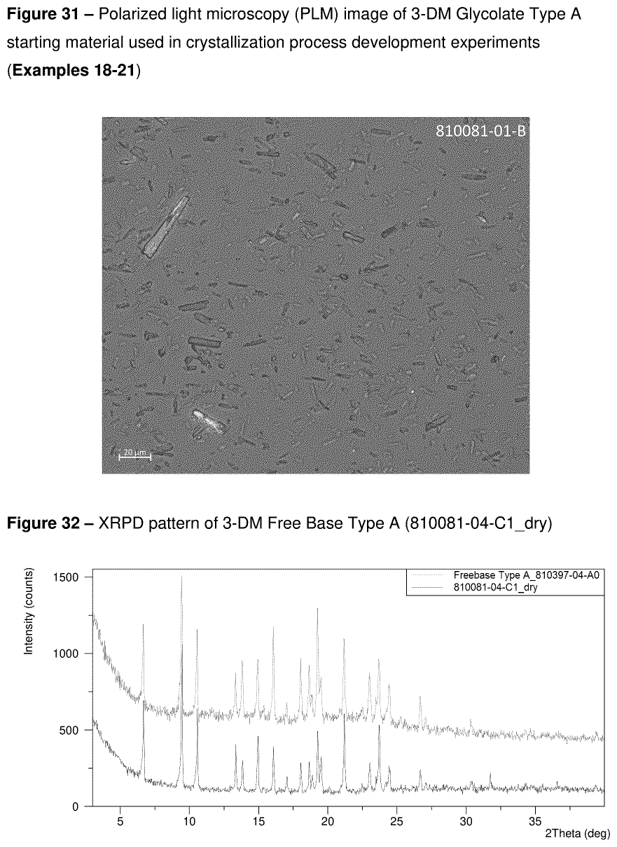

3-DM Glycolate Type A for use in crystallization process development experiments (see Examples 18-21) of this same material was characterized by XRPD, TGA, mDSC, ¹H NMR, PLM, PSD, and HPLC. XRPD confirmed that the material was Glycolate Type A. TGA/DSC revealed a weight loss of 1.2% up to 170° C. and a sharp endotherm at 221.9° C. (onset). ¹H NMR indicated that the molar ratio of acid to free base was 1.0:1 and that the residual solvent (IPA) to free base ratio was 0.08:1 (the corresponding TGA weight loss was 1.0 wt %). PLM as shown in FIG. 31 showed that the material consisted of short rod-like crystals. Particle size distribution (PSD) showed that the particle size D90 was 10.03 μm and a unimodal distribution was observed after sonication for 30 s under 30-Watt power (Table 46). HPLC purity was determined as 99.38% (area) and the area percentage of impurity 3-DCR was 0.07%, with detailed results summarized in Table 47.

TABLE 46
PSD results of 3-DM Glycolate Type A starting material.

| Sonication | MV | SD | D10 | D50 | D90 |
|---|---|---|---|---|---|
| time (s) | (μm) | (μm) | (μm) | (μm) | (μm) |
| 0 | 106.7 | 168.4 | 1.92 | 5.37 | 425.1 |
| 30 | 5.17 | 4.60 | 1.55 | 3.88 | 10.03 |

TABLE 47
HPLC data of 3-DM Glycolate Type A starting material.

| #Peak | RRT | Area (%) | #Peak | RRT | Area (%) |
|---|---|---|---|---|---|
| 1 | 0.69 | 0.08 | 5 | 0.91 (3-DCR) | 0.07 |
| 2 | 0.70 | 0.18 | 6 | 0.94 | 0.03 |
| 3 | 0.73 | 0.12 | 7 | 1.00 (3-DM) | 99.38 |
| 4 | 0.82 | 0.10 | 8 | 1.24 | 0.05 |

### Example 15: Solubility Measurements of 3-DM Glycolate Type A

In order to guide the optimization of the solution crystallization of 3-DM Glycolate Type A (see Examples 18-21), the solubility of 3-DM free base and 3-DM Glycolate Type A was measured in IPA and IPA/H₂O (19:1, v/v) at 5° C., 20° C., 40° C., and 60° C. Detailed experimental procedures were as follows.
1) Weighed about 50 mg of 3-DM free base or 25 mg of 3-DM Glycolate Type A into 3-mL glass vials. Added 1.0 mL of the corresponding solvent (IPA or IPA/H₂O (19:1, v/v)) into each glass vial to form a suspension.
2) Suspensions were mixed at 5° C., 20° C., 40° C., or 60° C. for 24 hrs.
3) After 24 hrs, suspensions were extracted into centrifugation tubes, followed by centrifugation (10,000 rpm, RT, 0.5 min).
4) The supernatants were filtered through a 0.45 μm PTFE membrane into 10-mL volumetric flasks, diluted to the volume by IPA, and these solutions were used for HPLC solubility tests. The residual solids were analyzed by XRPD.

XRPD showed that Free Base Type A (a crystalline form of 3-DM free base, see Example 16) and Glycolate Type A were obtained after solubility tests of 3-DM free base (amorphous) and 3-DM Glycolate Type A, respectively. Solubility results are summarized in Tables 48 and 49. The solubility of free base was much higher than that of Glycolate Type A in both IPA and IPA/H₂O (19:1, v/v) at each temperature. Therefore, both IPA and IPA/H₂O (19:1, v/v) were considered suitable for crystallization. In addition, a significant solubility decrease for Glycolate Type A was observed when the temperature was decreased from 60° C. to 5° C. Therefore, cooling was expected to further improve the yield. Furthermore, solubility of Free Base Type A in IPA/H₂O (19:1, v/v) was higher than IPA, so IPA/H₂O (19:1, v/v) was expected to result in a better volumetric efficiency. As there was not expected to be a significant difference in yield using either solvent, and IPA/H₂O (19:1, v/v) was used in previous formation of Glycolate Type A (see Example 7), it was selected as the preferred solvent.

TABLE 48
Solubility of 3-DM Free Base and Glycolate Type A in IPA.

| 3-DM Material | | Temp. | Solubility | |
|---|---|---|---|---|
| (Sample ID) | Experiment ID | (° C.) | (mg/mL) | Solid Form |
| Amorphous | 810081-04-A1 | 5 | 11.08 | Free Base |
| Free Base | | | | Type A |
| (810081-01-A) | 810081-04-B1 | 20 | 13.16 | Free Base |
| | | | | Type A |
| | 810081-45-C1 | 40 | 24.74^{#} | Free Base |
| | | | | Type A^{&} |
| | 810081-05-D1* | 60 | 56.70^{#} | Free Base |
| | | | | Type A |
| Glycolate | 810081-04-A3 | 5 | 2.29 | Glycolate |
| Type A | | | | Type A |
| (810397-64-B) | 810081-04-B3 | 20 | 2.93 | Glycolate |
| | | | | Type A |
| | 810081-45-C3 | 40 | 3.33^{#} | Glycolate |
| | | | | Type A^{&} |
| | 810081-45-D3 | 60 | 9.09^{#} | Glycolate |
| | | | | Type A^{&} |

*The initial loading concentration was 152 mg/mL.
^{#}Centrifugation (~8,000 rpm, 5 min) and filtration was performed at corresponding temperature, 40 or 60° C.
^{&}Solids from another experiment were collected for XRPD analysis.

TABLE 49
Solubility of 3-DM Free Base and Glycolate
Type A in IPA/H₂O (19:1, v/v).

| 3-DM Material | | Temp. | Solubility | |
|---|---|---|---|---|
| (Sample ID) | Experiment ID | (° C.) | (mg/mL) | Solid Form |
| Amorphous | 810081-04-A2 | 5 | 13.63 | Free Base |
| Free Base | | | | Type A |
| (810081-01-A) | 810081-04-B2 | 20 | 18.38 | Free Base |
| | | | | Type A |
| | 810081-45-C2 | 40 | 33.65^{#} | Free Base |
| | | | | Type A^{&} |
| | 810082-08-A1* | 60 | 73.94^{#} | Free Base |
| | | | | Type A^{&} |
| Glycolate | 810081-04-A4 | 5 | 5.30 | Glycolate |
| Type A | | | | Type A |
| (810397-64-B) | 810081-04-B4 | 20 | 5.88 | Glycolate |
| | | | | Type A |
| | 810081-45-C4 | 40 | 9.72^{#} | Glycolate |
| | | | | Type A^{&} |
| | 810081-45-D4 | 60 | 17.56^{#} | Glycolate |
| | | | | Type A^{&} |

*The initial loading concentration was ~75 mg/mL.
^{#}Centrifugation (~8,000 rpm, 5 min) and filtration was performed at corresponding temperature, 40 or 60° C.
^{&}Solids from another experiment were collected for XRPD analysis.

### Example 16: Characterization of 3-DM Free Base Type A

3-DM Free Base Type A was obtained via stirring of a slurry of amorphous 3-DM free base in IPA at 40° C. for one day and drying the resulting solid at RT under vacuum for 3 hrs. The XRPD pattern of the resulting material is displayed in FIG. 32. TGA revealed a weight loss of 2.2% up to 80° C. and a stepwise weight loss of 12.3% between 80° C. and 110° C. DSC showed a sharp endotherm at 94.6° C. (onset). 1H NMR showed that the molar ratio of residual solvent (IPA) to free base was 0.9:1 (corresponding TGA weight loss was 12.2 wt %, close to the second step weight loss). Based on data above, Free Base Type A was identified as an IPA solvate.

### Example 17: Solution Stability of 3-DM Free Base and 3-DM Glycolate Type A

To ensure that instability would not confound crystallization process development experiments (see Examples 18-21), solution stability of 3-DM free base and 3-DM Glycolate Type A in IPA and IPA/H₂O (19:1, v/v) at 20° C., 40° C., 60° C., and 65° C. was assessed. Detailed experimental procedures were as follows.
1) For each sample, weighed approximately 1 mg of material (3-DM free base or 3-DM Glycolate Type A) into a 3-mL glass vial. Added 1 mL of solvent to dissolve solid completely.
2) Stored solution samples at different temperatures (20° C., 40° C., 60° C., and 65° C.) for 38 hrs.
3) Tested HPLC purity for all solution samples.

Solution stability results are summarized in Table 50. No decrease in HPLC purity or increase in 3-DCR area percentage was observed under any condition, indicating good solution stability for amorphous 3-DM free base and 3-DM Glycolate Type A in IPA and IPA/H₂O (19:1, v/v) in the range of 20° C.-65° C.

TABLE 50
Solution stability results of 3-DM Free Base and 3-DM Glycolate Type A.

| | | HPLC purity | | | | |
|---|---|---|---|---|---|---|
| 3-DM | | of initial | | | | |
| Material | Solvent | sample | HPLC Final Purity/Initial Purity (%) | | | |
| (Sample ID) | (v/v) | (area %) | 20° C. | 40° C. | 60° C. | 65° C. |
| Amorphous | IPA | 99.57 | 100.3 | 100.4 | 100.3 | 100.3 |
| free base | IPA/H₂O | | 100.3 | 100.3 | 100.3 | 100.3 |
| (810081-01-A) | (19:1) | | | | | |
| Glycolate | IPA | 98.81 | 100.2 | 100.2 | 100.2 | 100.2 |
| Type A | IPA/H₂O | | 100.2 | 100.2 | 100.2 | 100.2 |
| (810397-64-B) | (19:1) | | | | | |

### Example 18: Preliminary Crystallization Process Development for 3-DM Glycolate Type A (300-mg Scale)

Preliminary crystallization experiments were performed in IPA/H₂O (19:1, v/v) using slow cooling method. Detailed procedures are described in Table 51. The process parameters and characterization results are summarized in Table 52. XRPD indicated that 3-DM Glycolate Type A was obtained in both batches prepared.

For Batch 810081-18-A2_dry. A weight loss of 1.7% up to 150° C., a weak endotherm at 202.8° C. (peak), and a sharp endotherm at 220.8° C. (onset) were observed in TGA/DSC. ¹H NMR showed that the molar ratio of acid/free base was 1.0:1, and residual IPA/free base was 0.04:1 (0.5 wt %). PLM showed needle-like or short rod-like crystals. HPLC purity was determined as 99.86% (area).

For Batch 810081-28-B: A weight loss of 0.8% up to 150° C. and a sharp endotherm at 223.0° C. (onset) were observed in TGA/DSC. ¹H NMR showed that the molar ratio of acid/free base was 1.0:1, and negligible solvent (IPA) was detected. PLM showed rod-like crystals. Results of PSD characterization are listed in Table 53. The particle size (D90) was 47.05 μm (after sonication for 30 s/30 Watt), and a unimodal distribution was observed before and after sonication. HPLC purity was determined as 99.89% (area).

The main differences between these two batches were initial temperature and seed loading. As the initial concentration was very close to the equilibrium solubility of Free Base Type A in IPA/H₂O (19:1, v/v) at 60° C. (73.94 mg/mL), for Batch 810081-28-B, the temperature was increased to 65° C. to prevent possible precipitation. For Batch 810081-18-A2_dry, no seeds were added and particles with small size were obtained. For batch 810081-28-B, ˜2% seeds were added for generation of crystals with larger particle size.

However, the yield of both batches was relatively low. For batches at only 300-mg scale, it was suspected that material loss during filtration and collection of solids might be very high. To confirm if a higher yield could be achieved on larger scale, additional experiments were conducted at 900-mg scale (see Example 19).

TABLE 51
Procedures for preliminary crystallization development
experiments for 3-DM Glycolate Type A at 300-mg scale.

| Batch No. | Experimental Procedure | |
|---|---|---|
| 810081-18- | 1. | Transfer 301.1 mg of free base (810081-01-A) into a |
| A2_dry | | 20-mL glass vial. Add 4.0 mL IPA/H₂O |
| | | (19:1, v/v). Magnetically stir at 60° C. for 30 |
| | | mins and filter the solution through 0.45 μm |
| | | PTFE membrane. |
| | 2. | Transfer 56.4 mg of glycolic acid into another 20-mL |
| | | glass vial. Add 4.0 mL IPA/H₂O (19:1, v/v) |
| | | to dissolve solids. |
| | 3. | Inject acid solution into the API solution. A suspension |
| | | was observed after ~1.4 mL of acid solution was |
| | | added. |
| | 4. | Slurry the suspension at 60° C. for 2 h, and cool |
| | | to 5° C. at 0.1° C./min. Keep isothermal at 5° C. |
| | 5. | Isolate solids by centrifugation (10,000 rpm, 2 mins). |
| | | Dry solids under vacuum at 50° C. and collect |
| | | solids after drying. |
| | 6. | A total of 257.0 mg of solids were obtained (Yield: |
| | | 71.9%). |
| 810081-28-B | 1. | Transfer 300.1 mg of free base (810081-01-A) into a |
| | | 20-mL glass vial. Add 4 mL IPA/H₂O (19:1, |
| | | v/v). Transfer the suspension to slurry at 65° C. |
| | | for 30 min before filtered with a 0.45 μm PTFE |
| | | membrane. |
| | 2. | Transfer 59.3 mg of glycolic acid into another 20-mL |
| | | glass vial. Add 3.0 mL IPA/H₂O (19:1, |
| | | v/v) to dissolve solids. |
| | 3. | Transfer the API solution into a 50-mL reactor and |
| | | stir at 65° C. |
| | 4. | Add acid solution into API solution over 30 min. |
| | 5. | Add 6.9 mg of Glycolate Type A (810081-18-A2_dry) |
| | | into the solution as seeds. Stir at 65° C. for 2 hrs. |
| | 6. | Cool to 25° C. at 0.1° C./min and keep slurry |
| | | at 25° C. for 15.5 hrs. Then cool to 5° C. at |
| | | 0.1° C./min and keep isothermal at 5° C. overnight. |
| | 7. | Collect solids by vacuum filtration. Dry solids at |
| | | 50° C. under vacuum for 4 h. A total of 214.0 mg |
| | | of solids were obtained (Yield: 59.6%). |

TABLE 52
Parameters and characterization results for preliminary
crystallization development experiments for 3-DM
Glycolate Type A at 300-mg scale.

| | | |
|---|---|---|
| Sample ID | 810081-18-A2_dry | 810081-28-B |

| | | | | |
|---|---|---|---|---|
| Scale (free base) | 300 | mg | 300 | mg |
| Initial temperature | 60° | C. | 65° | C. |
| Initial concentration | ~75 | mg/mL | ~75 | mg/mL |
| (free base) | | | | |

| | | |
|---|---|---|
| Seeds | NA | ~2% |
| XRPD | Glycolate Type A | Glycolate Type A |
| TGA weight loss (%) | 1.7 (to 150° C.) | 0.8 (to 150° C.) |
| DSC endotherm | 202.8*, 220.8 | 223.0 |

(° C., onset)

| | | |
|---|---|---|
| Molar ratio (acid/free | 1.0:1 | 1.0:1 |

base)

| | | |
|---|---|---|
| Residual IPA (wt %) | 0.5 | Not detected |
| Morphology | Needle-like or short | Rod-like |

rod-like

| | | |
|---|---|---|
| HPLC purity (area %) | 99.86 | 99.89 |
| Yield (%) | 71.9 | 59.6 |

*Peak temperature.

TABLE 53
PSD results of 3-DM Glycolate Type A (Batch 810081-28-B).

| Sonication | | | | | |
|---|---|---|---|---|---|
| Time (30 | MV | SD | D10 | D50 | D90 |
| Watt) | (μm) | (μm) | (μm) | (μm) | (μm) |
| 0 s | 28.68 | 18.45 | 7.51 | 19.96 | 58.66 |
| 30 s | 22.77 | 14.81 | 5.92 | 15.93 | 47.05 |

### Example 19: Additional Crystallization Process Development for 3-DM Glycolate Type A (900-mg Scale)

In order to improve the yield of 3-DM Glycolate Type A crystallization, two additional experiments were performed at 900-mg scale. Detailed procedures are described in Table 54. The process parameters and characterization results are summarized in Table 55. Glycolate Type A was obtained in both batches with ˜80% yield, which was higher than observed at 300-mg scale.

For Batch 810081-50-A: A weight loss of 0.7% up to 150° C., a weak endotherm at 204.5° C. (peak), and a sharp endotherm at 223.2° C. (onset) were observed in TGA/DSC. ¹H NMR showed that the molar ratio of acid/free base was 1.0:1, and residual IPA/free base was 0.01:1 (0.19 wt %). PLM showed that the sample was needle-like or rod-like crystals. HPLC purity was determined as 99.94% (area).

For Batch 810082-13-B: A weight loss of 1.0% up to 150° C., a weak endotherm at 205.2° C. (peak), and a sharp endotherm at 222.4° C. (onset) were observed in TGA/DSC. ¹H NMR showed that the molar ratio of acid/free base was 1.0:1, and residual IPA/free base was 0.01:1 (0.19 wt %). PLM showed that the sample was rod-like or short rod-like crystals. HPLC purity was determined as 99.91% (area).

The main difference between these two batches was seed loading. For Batch 810082-13-B, ˜2% seeds were added and the particle size was larger than that of Batch 810081-50-A, which was crystallized without seed addition. On the basis of these results, it was decided that seeding was likely to improve reproducibility at different scales and therefore, that seeds would be added at larger scale (see Example 20).

TABLE 54
Procedures for crystallization development experiments
for 3-DM Glycolate Type A at 900-mg scale.

| Batch No. | Experimental Procedure | |
|---|---|---|
| 810081-50-A | 1. | Transfer 900.8 mg of free base (810081-01-A) and 12 mL |
| | | IPA/H₂O (19:1, v/v) into a 20-mL glass vial. Magnetically stir |
| | | the suspension at 65° C. for 2 h. Clear API solution was |
| | | obtained. |
| | 2. | Transfer 180.4 mg of glycolic acid into another 20-mL glass |
| | | vial. Add 9 mL IPA/H₂O (19:1, v/v) to dissolve solid for acid |
| | | solution. |
| | 3. | Transfer the API solution into a 50-mL reactor and stir |
| | | at 65° C. |
| | 4. | Add acid solution into API solution over 1 h, and magnetically |
| | | stir (300 rpm) at 65° C. for 2 h. |
| | 5. | Cool to 5° C. at 0.1° C./min and keep slurry at 5° C. for 10 h. |
| | 6. | Sample 1 mL of suspension, filter it by 0.45 μm PTFE |
| | | membrane to obtain clear solution for concentration test |
| | | (3.76 mg/mL). |
| | 7. | After slurrying at 5° C. for 3.5 h, isolate suspension by |
| | | vacuum filtration. Wash the wet cake with 4 mL cold IPA. |
| | 8. | Dry solids under vacuum at RT for 13.5 h and then at 50° C. |
| | | for 2 h. |
| | 9. | A total of 815.0 mg of solids were obtained (Yield: 79.8%). |
| 810082-13-B | 1. | Transfer 899.7 mg of free base (810081-01-A) and 12 mL |
| | | IPA/H₂O (19:1, v/v) into a 20-mL glass vial. Magnetically stir |
| | | the suspension at 65° C. for 2 h. Clear API solution was |
| | | obtained. |
| | 2. | Transfer 180.2 mg of glycolic acid into another 20-mL glass |
| | | vial. Add 9 mL IPA/H₂O (19:1, v/v) to dissolve solid for acid |
| | | solution. |
| | 3. | Transfer the API solution into a 50-mL reactor and stir at |
| | | 65° C. |
| | 4. | Add 3 mL acid solution into API solution over 20 min. Then add |
| | | 18.2 mg of seeds (810081-18-A2_dry). A turbid suspension |
| | | was observed. |
| | 5. | Add 6 mL acid solution into API solution over 40 min. |
| | | Magnetically stir (300 rpm) at 65° C. for 2 h. |
| | 6. | Cool to 5° C. at 0.1° C./min and keep slurry at 5° C. for ~13.5 h. |
| | 7. | Isolate solids by vacuum filtration. Wash the wet cake by 4 mL |
| | | of cold IPA. |
| | 8. | Dry solids under vacuum at 50° C. for 2 h and then at RT for |
| | | 13 h. |
| | 9. | A total of 861.7 mg of solids were obtained (Yield: 78.7%). |

TABLE 55
Parameters and characterization results for crystallization development
experiments for 3-DM Glycolate Type A at 900-mg scale.

| Sample ID | 810081-50-A | | 810082-13-B | |
|---|---|---|---|---|
| Scale (free base) | 900 | mg | 900 | mg |
| Initial temperature | 65° | C. | 65° | C. |
| Initial concentration | ~75 | mg/mL | ~75 | mg/mL |

| (free base) | | |
|---|---|---|
| Seeds | NA | ~2% |
| XRPD | Glycolate Type A | Glycolate Type A |
| TGA weight loss (%) | 0.7 (to 150° C.) | 1.0 (to 150° C.) |
| DSC endotherm (° C., | 204.5*, 223.2 | 205.2*, 222.4 |
| onset) | | |
| Molar ratio (acid/free | 1.0:1 | 1.0:1 |
| base) | | |
| Residual IPA (wt %) | 0.19 | 0.19 |
| Morphology | Needle-like or rod-like | Rod-like or short rod-like |
| HPLC purity (area %) | 99.94 | 99.91 |
| Yield (%) | 79.8 | 78.7 |

*Peak temperature.

### Example 20: Crystallization of 3-DM Glycolate Type A at 5-g Scale

To further evaluate the robustness of the optimized crystallization method (see Example 19), an experiment at 5-g scale was conducted using the optimized parameters for solution crystallization of 3-DM Glycolate Type A, as follows:

| Initial free base concentration: | ~75 mg/mL in IPA/H₂O | |
|---|---|---|
| Initial acid concentration: | ~20 mg/mL in IPA/H₂O | |
| Volume ratio (free base/acid): | 4:3 | |
| Initial temperature: | 65° | C. |
| Seed loading: | ~2% | |
| Ending temperature: | 5° | C. |
| Cooling rate: | 0.1° | C./min |

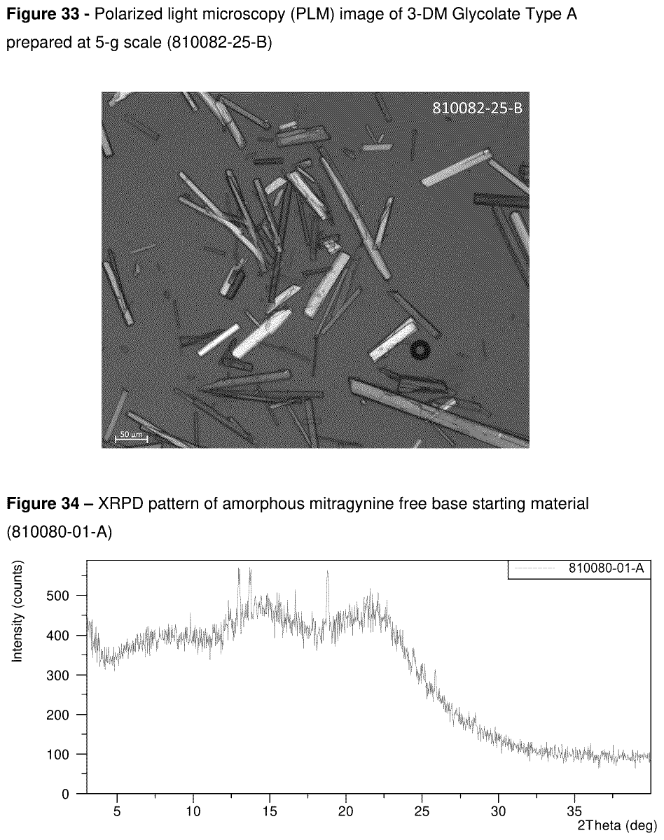

The detailed procedure is described in Table 56. XRPD showed that 3-DM Glycolate Type A was successfully obtained with a yield of 85.1%. TGA/DSC showed a weight loss of 1.0% up to 150° C., a weak endotherm at 206.9° C. (peak), and a sharp endotherm at 222.2° C. (onset). ¹H NMR showed that the molar ratio of acid/free base was 1.0:1, and residual IPA/free base was 0.02:1 (0.32 wt %). PLM showed that the product was rod-like crystals (FIG. 33). HPLC revealed a purity of 99.89% (area) and impurity 3-DCR was not detected (area percentage was ˜0.01%), as detailed in Table 57.

TABLE 56
Procedure for crystallization of 3-DM Glycolate Type A at 5-g scale.
Procedure

| | |
|---|---|
| 1. | Weigh 1.001 g of glycolic acid into a |
| | 100-mL glass vial. Add 50 mL IPA/H₂O |
| | (19:1, v/v) to dissolve solid for acid solution. |
| 2. | Add 5.010 g of free base (810081-01-A) and 66.7 mL |
| | IPA/H₂O (19:1, v/v) to a 250-mL reactor. |
| | Overhead stir the suspension at 65° C. (250 rpm) |
| | for 2 h for API solution. |
| 3. | Add 16 mL acid solution into API solution over 20 min. |
| | Then add 100.7 mg of seeds (810081-28-B). A turbid |
| | suspension was observed. |
| 4. | Add 34 mL acid solution into API solution over 40 min. |
| | Overhead stir (300 rpm) at 65° C. for 2 h. |
| 5. | Cool to 5° C. at 0.1° C./min and keep |
| | slurry at 5° C. for ~13.5 h. |
| 6. | Isolate solids by vacuum filtration. |
| | Wash the wet cake with ~10 mL cold IPA. |
| 7. | Dry solids under vacuum at RT for ~13.5 h |
| | and then at 50° C. for 3 h. |
| 8. | A total of 5.175 g of solids were obtained (Yield: 85.1%). |

A polarized light microscopy image of the 3-DM Glycolate Type A crystals prepared at 5-g scale (810082-25-B) is shown in FIG. 33.

TABLE 57
HPLC data of 3-DM Glycolate Type A prepared
at 5-g scale (810082-25-B).

| #Peak | RRT | Area (%) |
|---|---|---|
| 1 | 0.70 | 0.05 |
| 2 | 0.73 | 0.06 |
| 3 (3-DM) | 1.00 | 99.89 |

### Example 21: Crystallization of 3-DM Glycolate Type A Using IPA as Solvent (300-mg Scale)

Two crystallization experiments were conducted with 3-DM Glycolate using IPA as solvent via a slow cooling method. Detailed procedures are described in Table 58. XRPD revealed that Glycolate Type A was obtained in both batches, which were further characterized with the results summarized in Table 59.

For Batch 810082-18-B: A weight loss of 2.5% up to 150° C., a weak endotherm at 203.7° C. (peak), and a sharp endotherm at 221.8° C. (onset) were observed in TGA/DSC. ¹H NMR showed that the molar ratio of acid/free base was 1.0:1, and residual IPA/free base was 0.03:1 (0.6 wt %). PLM showed that the product was rod-like or short rod-like crystals.

For Batch 810082-20-A: A weight loss of 1.3% up to 150° C., a weak endotherm at 203.9° C. (peak), and a sharp endotherm at 222.2° C. (onset) were observed in TGA/DS. ¹H NMR showed that the molar ratio of acid/free base was 1.0:1, and residual IPA/free base was 0.04:1 (0.7 wt %). PLM showed that the product was rod-like or short rod-like crystals.

When switching to IPA, the residual solvent increased compared to batches prepared using IPA/H₂O (19:1, v/v). In addition, the IPA/H₂O (19:1, v/v) procedure could be run at the higher concentration of 75 mg/mL with full dissolution, whereas a reduction to 50 mg/mL was required in the IPA procedure.

TABLE 58
Procedures for crystallization of 3-DM Glycolate Type A from IPA.

| Batch No. | Experimental Procedure | |
|---|---|---|
| 810082-18-B | 1. | Add 300.6 mg of free base (810081-01-A) and 4 mL IPA into |
| | | a 20-mL glass vial. Magnetically stir the suspension at 65° C. |
| | | for 1 h. |
| | 2. | Additional 2 mL of IPA was added as some solids were not |
| | | dissolved. Magnetically stir at 65° C. for additional 2 h for API |
| | | solution. |
| | 3. | Transfer 68.0 mg of glycolic acid into another 20-mL glass vial. |
| | | Add 3.4 mL IPA to dissolve solid for acid solution. |
| | 4. | Transfer the API solution into a 50-mL reactor and stir at |
| | | 65° C. |
| | 5. | Add 1 mL acid solution into API solution over 7 min. Then add |
| | | 6.0 mg of seeds (810081-18-A2_dry). A turbid suspension was |
| | | observed. |
| | 6. | Add 2 mL acid solution into API solution over 13 min. |
| | | Magnetically stir (300 rpm) at 65° C. for 2 h. |
| | 7. | Cool to 5° C. at 0.1° C./min and keep slurry at 5° C. for ~13.5 h. |
| | 8. | Isolate solids by vacuum filtration. Wash the wet cake with ~4 |
| | | mL cold IPA. |
| | 9. | Dry solids under vacuum at 50° C. for 2 h and then at RT for |
| | | 13 h. |
| | 10. | A total of 270.2 mg of solids were obtained (Yield: 73.8%). |
| 810082-20-A | 1. | Add 300.4 mg of free base (810081-01-A) and 4 mL IPA into |
| | | a 50-mL reactor. Magnetically stir at 65° C. for ~1.5 h. Solids |
| | | were not dissolved completely. |
| | 2. | Transfer 67.9 mg of glycolic acid into another 20-mL glass vial. |
| | | Add 3.4 mL IPA to dissolve solid for acid solution. |
| | 3. | Add 1 mL acid solution into API suspension over 7 min. Then |
| | | add 6.1 mg of seeds (810081-18-A2_dry). A turbid suspension |
| | | was observed. |
| | 4. | Add 2 mL acid solution into API solution over 13 min. |
| | | Magnetically stir (300 rpm) at 65° C. for 2 h. |
| | 5. | Cool to 5° C. at 0.1° C./min and keep slurry at 5° C. for ~ 13.5 h. |
| | 6. | Isolate solids by vacuum filtration. Wash the wet cake with ~4 |
| | | mL cold IPA. |
| | 7. | Dry solids under vacuum at 50° C. for 2 h and then at RT for |
| | | 13 h. |
| | 8. | A total of 300.2 mg of solids were obtained (Yield: 82.2%). |

TABLE 59
Parameters and characterization results of 3-DM
Glycolate Type A crystallized from IPA.

| Sample ID | 810082-18-B | | 810082-20-A | |
|---|---|---|---|---|
| Scale (free base) | 300 | mg | 300 | mg |
| Initial temperature | 65° | C. | 65° | C. |
| Initial concentration | ~50 | mg/mL | ~75 | mg/mL |

| (free base) | | |
|---|---|---|
| Seeds | ~2% | ~2% |
| XRPD | Glycolate Type A | Glycolate Type A |
| TGA weight | 2.5 (to 150° C.) | 1.3 (to 150° C.) |
| loss (%) | | |
| DSC endotherm | 203.7*, 221.8 | 203.9*, 222.2 |
| (° C., onset) | | |
| Sample ID | 810082-18-B | 810082-20-A |
| Molar ratio | 1.0:1 | 1.0:1 |
| (acid/free base) | | |
| Residual | 0.6 | 0.7 |
| IPA (wt %) | | |
| Morphology | Rod-like or short rod-like | Rod-like or short rod-like |
| HPLC purity | 99.91 | 99.91 |
| (area %) | | |
| Yield (%) | 73.8 | 82.2 |

*Peak temperature.

### Example 22. Characterization of Mitragynine Free Base and Crude Alkaloid Extract for Salt Preparation

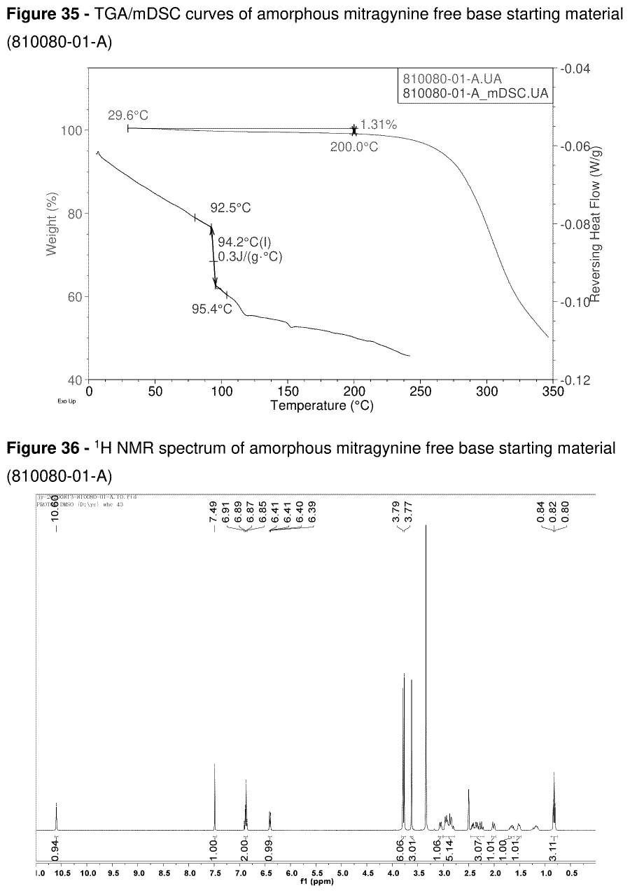

Pure mitragynine free base (810080-01-A) for use in salt preparation experiments was commercially obtained. The material was characterized by XRPD, TGA, mDSC, ¹H NMR, and HPLC. XRPD showed that the material was amorphous, with several weak peaks (FIG. 34). The TGA curve showed a weight loss of 1.3% up to 200° C. and mDSC curve revealed a possible glass transition (Tg) signal at 94.2° C. (middle temperature) (FIG. 35). ¹H NMR confirmed the structure of the material (FIG. 36). As shown in Table 60, HPLC purity was determined as 98.98% (area) and the content of corynantheidine (CR) impurity was determined as 0.48% (area).

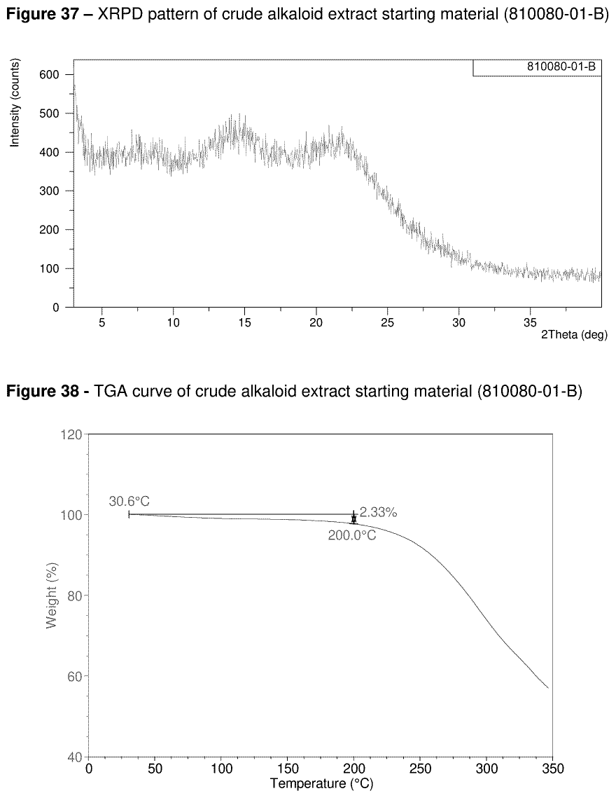

Crude alkaloid extract of Mitragyna speciosa (810080-01-B) was commercially obtained and characterized by XRPD, TGA, and HPLC. XRPD showed that the material was amorphous (See FIG. 37). TGA showed a weight loss of 2.3% up to 200° C. (See FIG. 38). The weight content of mitragynine was found to be 61.0% by HPLC (75.94 area %) using pure mitragynine (810080-01-A) as a reference standard. The CR content was found to be 1.19% (area).

TABLE 60
Individual impurities of amorphous mitragynine free base
starting material (810080-01-A) as determined by HPLC.

| #Peak | RRT | Area(%) |
|---|---|---|
| 1 | 0.69 | 0.11 |
| 2 | 0.86 | 0.06 |
| 3 | 0.90 (CR) | 0.48 |
| 4 | 1.00 (mitragynine) | 98.98 |
| 5 | 1.06 | 0.05 |
| 6 | 1.17 | 0.06 |
| 7 | 1.20 | 0.11 |
| 8 | 1.23 | 0.05 |
| 9 | 1.29 | 0.10 |

### Example 23: Preparation and Characterization of Mitragynine Salts

Using mitragynine free base as the starting material, mitragynine Fumarate Type A, L-Lactate Type A, Glycolate Type A, Succinate Type A, and Mesylate Type A were prepared at 500-mg or 750-mg scale using procedures analogous to those used to prepare the corresponding 3-DM salts. The detailed preparation procedures are described in Table 61.

TABLE 61
Preparation procedures for mitragynine salts.

| Mitragynine Salt | | |
|---|---|---|
| Lead | | |
| (Sample ID) | Procedure | |
| Fumarate Type A | 1. | Transfer 500.9 mg of free base (810080-01-A) and 149.9 |
| (810080-06-B1) | | mg of fumaric acid into a 20-mL glass vial. |
| | 2. | Add 12 mL isopropanol IPA/H₂O (4:1, v/v) to form a |
| | | suspension. Magnetically stir (~1000 rpm) at RT for 2 |
| | | days. |
| | 3. | Isolate solids by vacuum filtration. Dry solids at RT under |
| | | vacuum for ~15 h. |
| | 4. | A total of 439.7 mg of material was obtained (Yield: |
| | | 67.6%). |
| L-Lactate Type A | 1. | Transfer 126.4 mg of L-lactic acid into a 20-mL glass vial, |
| (810080-06-B2) | | add 8 mL IPA/H₂O (4:1, v/v) to dissolve acid. |
| | 2. | Transfer 499.5 mg of free base (810080-01-A) into the acid |
| | | solution. Magnetically stir (~1000 rpm) at RT for 2 days. |
| | 3. | Isolate solids by vacuum filtration. Dry solids at RT under |
| | | vacuum for ~15 h. |
| | 4. | A total of 266.8 mg of material was obtained (Yield: 42.6%). |
| Glycolate Type A | 1. | Transfer 499.8 mg of free base (810080-01-A) and 98.4 mg |
| (810080-07-B3) | | of glycolic acid into a 20-mL glass vial. |
| | 2. | Add 12 mL IPA/H₂O (4:1, v/v) to form a suspension. |
| | | Magnetically stir (~1000 rpm) at RT for 2 days. |
| | 3. | Isolate solids by vacuum filtration. Dry solids at RT under |
| | | vacuum for ~15 h. |
| | 4. | A total of 203.4 mg of material was obtained (Yield: 34.0%, |
| | | with ID: 810080-06-B3). |
| | 5. | Weigh 250.3 mg of free base (810080-01-A) and 49.6 mg |
| | | of glycolic acid into the filtrate. Magnetically stir (~1000 |
| | | rpm) at RT for 2 days. |
| | 6. | Add the dried sample (810080-06-B3) into the suspension, |
| | | magnetically stir (~1000 rpm) at RT for 4 h. |
| | 7. | Isolate solids by vacuum filtration. Dry solids at 50° C. under |
| | | vacuum for ~2 h. |
| | 8. | A total of 427.6 mg of material was obtained (Yield: 47.6%). |
| Succinate Type A | 1. | Transfer 500.8 mg of free base (810080-01-A) and 151.4 |
| (810080-06-B4) | | mg of succinic acid into a 20-mL glass vial. |
| | 2. | Add 12 mL IPA/H₂O (4:1, v/v) to form a suspension. |
| | | Magnetically stir (~1000 rpm) at RT for 2 days. |
| | 3. | Isolate solids by vacuum filtration. Dry solids at RT under |
| | | vacuum for ~15 h. |
| | 4. | A total of 362.7 mg of material was obtained |
| | | (Yield: 55.6%). |
| Mesylate Type A | 1. | Transfer 126.7 mg of methanesulfonic acid into a 20-mL |
| (810080-06-B5) | | glass vial, add 10 mL ethyl acetate (EtOAc) to dissolve |
| | | acid. |
| | 2. | Transfer 501.0 mg of free base (810080-01-A) into the acid |
| | | solution. Magnetically stir (~1000 rpm) at RT for 2 days. |
| | 3. | Isolate solids by vacuum filtration. Dry solids at RT under |
| | | vacuum for ~15 h. |
| | 4. | A total of 468.0 mg of material was obtained |
| | | (Yield: 74.6%). |

XRPD revealed that the mitragynine salts obtained had similar crystalline properties to those obtained from the deuterated material, 3-DM (Table 62). The full XRPD traces are also shown in FIGS. 39-43.

TABLE 62
XRPD copper radiation results for mitragynine salts.

| Mitragynine | | Height | FWHM | d-spacing | |
|---|---|---|---|---|---|
| Salt | Pos. [°2θ] | [cts] | Left [°2θ] | [Å] | Rel. Int. |
| Fumarate | 8.30 | 877.10 | 0.1023 | 10.65 | 100.00 |
| Type A | 9.47 | 149.70 | 0.1023 | 9.34 | 17.07 |
| | 14.29 | 311.81 | 0.1535 | 6.20 | 35.55 |
| | 15.06 | 83.30 | 0.1535 | 5.88 | 9.50 |
| | 15.47 | 64.83 | 0.1535 | 5.73 | 7.39 |
| | 16.03 | 211.03 | 0.1279 | 5.53 | 24.06 |
| | 16.73 | 234.50 | 0.1023 | 5.30 | 26.74 |
| | 17.33 | 513.93 | 0.1279 | 5.12 | 58.59 |
| | 18.55 | 338.24 | 0.1023 | 4.78 | 38.56 |
| | 19.06 | 765.72 | 0.1023 | 4.66 | 87.30 |
| | 19.22 | 788.22 | 0.1023 | 4.62 | 89.87 |
| | 19.89 | 95.70 | 0.1535 | 4.46 | 10.91 |
| | 21.19 | 221.58 | 0.1023 | 4.19 | 25.26 |
| | 21.44 | 238.21 | 0.1023 | 4.14 | 27.16 |
| | 22.22 | 175.89 | 0.1279 | 4.00 | 20.05 |
| | 22.89 | 204.35 | 0.1279 | 3.89 | 23.30 |
| | 23.22 | 299.25 | 0.1023 | 3.83 | 34.12 |
| | 23.61 | 265.88 | 0.1023 | 3.77 | 30.31 |
| | 24.51 | 126.44 | 0.1279 | 3.63 | 14.42 |
| | 25.20 | 373.37 | 0.1791 | 3.53 | 42.57 |
| | 25.62 | 564.11 | 0.1279 | 3.48 | 64.32 |
| | 26.81 | 199.64 | 0.1279 | 3.33 | 22.76 |
| | 28.80 | 227.71 | 0.1023 | 3.10 | 25.96 |
| | 30.95 | 198.61 | 0.1535 | 2.89 | 22.64 |
| | 34.11 | 20.76 | 0.6140 | 2.63 | 2.37 |
| L-Lactate | 7.01 | 1984.77 | 0.1023 | 12.61 | 46.69 |
| Type A | 10.12 | 4250.55 | 0.1023 | 8.74 | 100.00 |
| | 10.87 | 836.75 | 0.1023 | 8.14 | 19.69 |
| | 11.20 | 3069.37 | 0.1279 | 7.90 | 72.21 |
| | 13.17 | 972.58 | 0.1023 | 6.72 | 22.88 |
| | 13.94 | 298.46 | 0.1023 | 6.35 | 7.02 |
| | 15.06 | 347.19 | 0.1023 | 5.88 | 8.17 |
| | 15.57 | 873.48 | 0.1023 | 5.69 | 20.55 |
| | 15.90 | 2338.73 | 0.1791 | 5.57 | 55.02 |
| | 17.88 | 1729.79 | 0.1535 | 4.96 | 40.70 |
| | 18.25 | 456.60 | 0.1023 | 4.86 | 10.74 |
| | 18.95 | 727.87 | 0.0768 | 4.68 | 17.12 |
| | 19.44 | 1859.59 | 0.1023 | 4.57 | 43.75 |
| | 19.91 | 1020.38 | 0.1023 | 4.46 | 24.01 |
| | 20.80 | 2959.52 | 0.1279 | 4.27 | 69.63 |
| | 21.18 | 199.50 | 0.1023 | 4.20 | 4.69 |
| | 21.76 | 142.00 | 0.1535 | 4.08 | 3.34 |
| | 22.42 | 2886.58 | 0.1279 | 3.96 | 67.91 |
| | 22.86 | 373.87 | 0.1535 | 3.89 | 8.80 |
| | 23.44 | 231.96 | 0.0768 | 3.79 | 5.46 |
| | 23.77 | 382.95 | 0.1023 | 3.74 | 9.01 |
| | 24.53 | 543.69 | 0.1279 | 3.63 | 12.79 |
| | 24.90 | 2134.64 | 0.1279 | 3.58 | 50.22 |
| | 25.80 | 80.85 | 0.2047 | 3.45 | 1.90 |
| | 27.09 | 590.27 | 0.1023 | 3.29 | 13.89 |
| | 27.34 | 631.24 | 0.1023 | 3.26 | 14.85 |
| | 28.16 | 202.54 | 0.1279 | 3.17 | 4.77 |
| | 29.45 | 367.99 | 0.1279 | 3.03 | 8.66 |
| | 30.73 | 88.24 | 0.3070 | 2.91 | 2.08 |
| | 31.36 | 461.46 | 0.1279 | 2.85 | 10.86 |
| | 33.97 | 197.90 | 0.1023 | 2.64 | 4.66 |
| | 35.72 | 148.09 | 0.2047 | 2.51 | 3.48 |
| | 37.41 | 66.97 | 0.2558 | 2.40 | 1.58 |
| | 38.12 | 42.44 | 0.3070 | 2.36 | 1.00 |
| Glycolate | 7.09 | 2227.09 | 0.1023 | 12.46 | 59.02 |
| Type A | 10.16 | 3773.50 | 0.1023 | 8.71 | 100.00 |
| | 11.29 | 2780.95 | 0.1023 | 7.84 | 73.70 |
| | 13.23 | 1002.77 | 0.1023 | 6.69 | 26.57 |
| | 14.14 | 569.61 | 0.1023 | 6.26 | 15.09 |
| | 15.10 | 226.01 | 0.1023 | 5.87 | 5.99 |
| | 15.74 | 1100.63 | 0.0768 | 5.63 | 29.17 |
| | 16.01 | 2026.54 | 0.0768 | 5.53 | 53.70 |
| | 18.03 | 1749.18 | 0.1279 | 4.92 | 46.35 |
| | 18.54 | 640.19 | 0.1023 | 4.79 | 16.97 |
| | 18.90 | 243.68 | 0.1023 | 4.69 | 6.46 |
| | 19.22 | 710.87 | 0.0768 | 4.62 | 18.84 |
| | 19.54 | 1864.83 | 0.0768 | 4.54 | 49.42 |
| | 19.72 | 1408.53 | 0.0768 | 4.50 | 37.33 |
| | 20.36 | 1575.92 | 0.1023 | 4.36 | 41.76 |
| | 20.93 | 3005.06 | 0.1023 | 4.24 | 79.64 |
| | 22.62 | 2357.70 | 0.1279 | 3.93 | 62.48 |
| | 23.26 | 297.77 | 0.1023 | 3.82 | 7.89 |
| | 23.52 | 246.96 | 0.0768 | 3.78 | 6.54 |
| | 23.76 | 284.00 | 0.1023 | 3.74 | 7.53 |
| | 24.05 | 457.06 | 0.1023 | 3.70 | 12.11 |
| | 24.90 | 563.37 | 0.1279 | 3.58 | 14.93 |
| | 25.20 | 2346.48 | 0.1279 | 3.53 | 62.18 |
| | 25.94 | 100.05 | 0.1535 | 3.43 | 2.65 |
| | 27.63 | 1346.88 | 0.1023 | 3.23 | 35.69 |
| | 28.30 | 161.35 | 0.1023 | 3.15 | 4.28 |
| | 29.12 | 93.42 | 0.1535 | 3.07 | 2.48 |
| | 29.64 | 149.00 | 0.2047 | 3.01 | 3.95 |
| | 30.15 | 670.34 | 0.1023 | 2.96 | 17.76 |
| | 30.64 | 193.78 | 0.1023 | 2.92 | 5.14 |
| | 31.66 | 576.95 | 0.1023 | 2.83 | 15.29 |
| | 32.24 | 183.42 | 0.1279 | 2.78 | 4.86 |
| | 34.20 | 147.65 | 0.1535 | 2.62 | 3.91 |
| | 35.16 | 131.39 | 0.1023 | 2.55 | 3.48 |
| | 36.03 | 132.20 | 0.1023 | 2.49 | 3.50 |
| | 36.63 | 124.79 | 0.1023 | 2.45 | 3.31 |
| Succinate | 6.24 | 979.78 | 0.1023 | 14.17 | 4.93 |
| Type A | 8.47 | 19866.64 | 0.1023 | 10.44 | 100.00 |
| | 9.14 | 570.92 | 0.1279 | 9.67 | 2.87 |
| | 9.62 | 1525.98 | 0.1023 | 9.20 | 7.68 |
| | 10.07 | 468.63 | 0.1023 | 8.78 | 2.36 |
| | 13.48 | 87.24 | 0.1535 | 6.57 | 0.44 |
| | 14.36 | 2064.94 | 0.1023 | 6.17 | 10.39 |
| | 15.66 | 1012.50 | 0.1023 | 5.66 | 5.10 |
| | 16.13 | 1233.90 | 0.1023 | 5.50 | 6.21 |
| | 16.95 | 573.33 | 0.1023 | 5.23 | 2.89 |
| | 17.56 | 4829.41 | 0.1023 | 5.05 | 24.31 |
| | 18.72 | 618.40 | 0.1023 | 4.74 | 3.11 |
| | 19.27 | 4490.83 | 0.1023 | 4.61 | 22.60 |
| | 21.21 | 187.16 | 0.1023 | 4.19 | 0.94 |
| | 21.71 | 1704.95 | 0.1023 | 4.09 | 8.58 |
| | 22.33 | 888.53 | 0.1023 | 3.98 | 4.47 |
| | 23.13 | 1576.63 | 0.1023 | 3.84 | 7.94 |
| | 23.72 | 346.57 | 0.1023 | 3.75 | 1.74 |
| | 24.49 | 183.17 | 0.1023 | 3.63 | 0.92 |
| | 25.03 | 676.61 | 0.1023 | 3.56 | 3.41 |
| | 25.33 | 1045.48 | 0.0768 | 3.52 | 5.26 |
| | 25.52 | 1637.15 | 0.1023 | 3.49 | 8.24 |
| | 25.93 | 1819.07 | 0.1279 | 3.44 | 9.16 |
| | 26.97 | 339.78 | 0.0768 | 3.31 | 1.71 |
| | 28.63 | 200.76 | 0.1023 | 3.12 | 1.01 |
| | 29.08 | 491.71 | 0.1023 | 3.07 | 2.48 |
| | 30.52 | 139.21 | 0.1279 | 2.93 | 0.70 |
| | 31.27 | 1486.70 | 0.1279 | 2.86 | 7.48 |
| | 31.56 | 322.01 | 0.1023 | 2.83 | 1.62 |
| | 32.97 | 249.55 | 0.1279 | 2.72 | 1.26 |
| | 34.22 | 179.42 | 0.1023 | 2.62 | 0.90 |
| | 34.55 | 163.67 | 0.1023 | 2.60 | 0.82 |
| | 37.03 | 94.16 | 0.3070 | 2.43 | 0.47 |
| | 39.05 | 158.57 | 0.1023 | 2.31 | 0.80 |
| Mesylate | 6.71 | 2834.93 | 0.1023 | 13.18 | 100.00 |
| Type A | 8.21 | 375.62 | 0.1023 | 10.77 | 13.25 |
| | 10.03 | 218.11 | 0.1023 | 8.82 | 7.69 |
| | 11.59 | 1208.66 | 0.1023 | 7.64 | 42.63 |
| | 12.95 | 161.80 | 0.1279 | 6.84 | 5.71 |
| | 13.38 | 809.76 | 0.1023 | 6.62 | 28.56 |
| | 14.94 | 866.79 | 0.1023 | 5.93 | 30.58 |
| | 15.31 | 438.33 | 0.1023 | 5.79 | 15.46 |
| | 16.36 | 220.09 | 0.1023 | 5.42 | 7.76 |
| | 16.71 | 1770.92 | 0.1023 | 5.30 | 62.47 |
| | 17.37 | 1374.36 | 0.1023 | 5.10 | 48.48 |
| | 18.31 | 256.80 | 0.0768 | 4.84 | 9.06 |
| | 18.62 | 932.18 | 0.1023 | 4.76 | 32.88 |
| | 18.93 | 1154.31 | 0.1023 | 4.69 | 40.72 |
| | 19.79 | 564.35 | 0.1023 | 4.49 | 19.91 |
| | 20.07 | 909.38 | 0.1023 | 4.42 | 32.08 |
| | 21.17 | 308.16 | 0.1023 | 4.20 | 10.87 |
| | 21.44 | 348.36 | 0.1023 | 4.14 | 12.29 |
| | 22.24 | 475.02 | 0.1023 | 4.00 | 16.76 |
| | 22.71 | 576.59 | 0.1023 | 3.92 | 20.34 |
| | 22.96 | 319.53 | 0.1023 | 3.87 | 11.27 |
| | 23.71 | 276.64 | 0.0768 | 3.75 | 9.76 |
| | 24.17 | 378.94 | 0.0768 | 3.68 | 13.37 |
| | 24.42 | 503.38 | 0.1023 | 3.65 | 17.76 |
| | 24.64 | 227.58 | 0.1023 | 3.61 | 8.03 |
| | 25.08 | 366.85 | 0.1023 | 3.55 | 12.94 |
| | 25.99 | 920.51 | 0.1279 | 3.43 | 32.47 |
| | 26.83 | 212.37 | 0.1023 | 3.32 | 7.49 |
| | 27.08 | 260.83 | 0.1023 | 3.29 | 9.20 |
| | 28.51 | 206.55 | 0.1279 | 3.13 | 7.29 |
| | 30.08 | 88.12 | 0.1535 | 2.97 | 3.11 |
| | 32.85 | 141.62 | 0.1023 | 2.73 | 5.00 |
| | 33.73 | 73.68 | 0.1535 | 2.66 | 2.60 |
| | 37.05 | 44.01 | 0.3070 | 2.43 | 1.55 |

All mitragynine salts obtained were further characterized by TGA, DSC, 1H NMR, PLM, and HPLC, with the results summarized in Table 63.

TABLE 63
Characterization results of mitragynine salts.

| | TGA | | | | | | Water |
|---|---|---|---|---|---|---|---|
| Mitragynine | weight | DSC | Molar | | | | Content |
| Salt | loss | endotherm | ratio | PLM | Purity | CR | by KF |
| (Sample ID) | (%) | (° C., onset) | (acid/base) | Appearance | (area %) | (area %) | (%) |
| Fumarate | 3.3 | 253.3 | 1:1 | fine | 99.41 | 0.41 | 0.47 |
| Type A | (to 150° | | | particles | | | |
| (810080- | C.) | | | | | | |
| 06-B1) | | | | | | | |
| Glycolate | 2.3 | 220.3 | 1.1:1 | rod-like | 99.89 | 0.06 | 0.60 |
| Type A | (to 150° | | | crystals | | | |
| (810080- | C.) | | | | | | |
| 07-B3) | | | | | | | |
| L-Lactate | 2.6 | 226.2 | 1:1 | short rod- | 99.81 | 0.11 | 0.31 |
| Type A | (to 150° | | | like | | | |
| (810080- | C.) | | | crystals | | | |
| 06-B2) | | | | | | | |
| Succinate | 3.9 | 197.7*, | 1.1:1 | needle- | 99.63 | 0.26 | 0.42 |
| Type A | (to 150° | 201.9* | | like | | | |
| (810080- | C.) | | | crystals | | | |
| 06-B4) | | | | | | | |
| Mesylate | 1.6 | 274.9 | 1:1 | fine | 99.09 | 0.48 | 0.47 |
| Type A | (to 150° | | | particles | | | |
| (810080- | C.) | | | | | | |
| 06-B5) | | | | | | | |

*Peak temperature.

### Example 24: Solubility of Mitragynine Salts

Solubility of mitragynine Fumarate Type A, L-Lactate Type A, Glycolate Type A, Succinate Type A, and Mesylate Type A in water and bio-relevant media (SGF, FaSSIF, and FeSSIF) was measured after 4 h at 37° C. Bio-relevant media were prepared in the same manner as Example 4. Solids were suspended in media at a solid loading of 10 mg/ml (calculated based on free base). The suspensions were agitated on a rolling incubator at 25 rpm at 37° C., prior to sampling at 4 h. At sampling time point, suspensions were separated by centrifugation (˜10,000 rpm, 37° C., 3 min) prior to filtering the supernatants through 0.45 μm PTFE membranes. The filtrates were analyzed for HPLC solubility and pH, and the residual solids were used for XRPD analysis. Results are summarized in Table 64. No form change was observed by XRPD for any of the salts in any of the media after 4 h.

TABLE 64
Summary of solubility results of mitragynine salts.

| Salt lead | H₂O | | | SGF | | | FaSSIF | | | FeSSIF | | |
|---|---|---|---|---|---|---|---|---|---|---|---|---|
| (810080-) | S | pH | FC | S | pH | FC | S | pH | FC | S | pH | FC |
| Fumarate | 0.40 | 4.3 | No | 1.28 | 1.9 | No | 2.31 | 5.6 | No | 1.91 | 4.9 | No |
| Type A | | | | | | | | | | | | |
| (06-B1) | | | | | | | | | | | | |
| L-Lactate | 1.87 | 5.6 | No | 7.04 | 3.0 | No | 0.97 | 6.1 | No | 2.28 | 4.9 | No |
| Type A | | | | | | | | | | | | |
| (06-B2) | | | | | | | | | | | | |
| Glycolate | 7.88 | 5.3 | No | 7.76 | 3.4 | — | 1.03 | 6.1 | No | 2.22 | 4.9 | No |
| Type A | | | | | | | | | | | | |
| (07-B3) | | | | | | | | | | | | |
| Succinate | 3.14 | 4.9 | No | 7.63 | 3.4 | No | 4.63 | 5.3 | No | 2.81 | 4.9 | No |
| Type A | | | | | | | | | | | | |
| (06-B4) | | | | | | | | | | | | |
| Mesylate | 13.83* | 4.6 | No | 8.11 | 1.8 | — | 1.72 | 5.9 | No | 2.36 | 4.9 | No |
| Type A | | | | | | | | | | | | |
| (06-B5) | | | | | | | | | | | | |

S: Solubility, mg/mL, calculated based on free base.
FC: Form change.
*The initial loading concentration was ~20 mg/mL (calculated based on free base).
—: Only limited amount of gel-like sample was obtained after solubility experiment. Therefore, XRPD was not conducted.

### Example 25: Hygroscopicity of Mitragynine Salts

To investigate the solid form stability and hygroscopicity of mitragynine salts as a function of humidity, DVS isotherm plots of mitragynine Fumarate Type A, L-Lactate Type A, Glycolate Type A, Succinate Type A, and Mesylate Type A were collected at 25° C. between 0 and 95% RH. After DVS test, samples were characterized by XRPD. The results are summarized in Table 65. No form change was observed by XRPD for any of the salts after DVS test.

TABLE 65
Summary of DVS results for mitragynine salts.

| Mitragynine Salt | Water uptake | Form |
|---|---|---|
| (Sample ID) | at 80% RH | change |
| Fumarate Type A | 0.18% | No |
| (810080-06-B1) | | |
| L-Lactate Type A | 0.03% | No |
| (810080-46-A1_dry)* | | |
| Glycolate Type A | 0.25% | No |
| (810080-07-B3) | | |
| Succinate Type A | 0.09% | No |
| (810080-06-B4) | | |
| Mesylate Type A | 0.40% | No |
| (810080-06-B5) | | |

*The remaining L-Lactate Type A (810080-06-B2) was not sufficient for DVS test, so another batch of L-Lactate Type A (810080-46-A1_dry) was used.

### Example 26: Chemical and Solid Form Stability of Mitragynine Salts

The solid state stability of mitragynine Fumarate Type A, L-Lactate Type A, Glycolate Type A, Succinate Type A, and Mesylate Type A was evaluated under conditions of 25° C./60% RH and 40° C./75% RH for 1 week. Each sample was placed in a glass vial, sealed by parafilm with several holes, and stored under the designated tested condition. After one week, samples were taken for XRPD, KF, and HPLC purity test. The results are summarized in Table 66. No purity decrease, form change, or water content difference was observed for any of the salts after storage under either condition for one week.

TABLE 66
Summary of physicochemical stability evaluation of mitragynine salts.

| | | | 25° C./60% RH/1 week | | | 40° C./75% RH/1 week | | |
|---|---|---|---|---|---|---|---|---|
| Mitragynine | Initial | | Purity/Initial | | | Purity/Initial | | |
| Salt | Purity | KF | purity | KF | Form | purity | KF | Form |
| (810080-) | (area %) | (wt %) | (%) | (wt %) | change | (%) | (wt %) | change |
| Fumarate | 99.41 | 0.47 | 99.9 | 0.24 | No | 100.2 | 0.34 | No |
| Type A | | | | | | | | |
| (06-B1) | | | | | | | | |
| L-Lactate | 99.81 | 0.31 | 100.1 | 0.43 | No | 100.1 | 0.68 | No |
| Type A | | | | | | | | |
| (06-B2) | | | | | | | | |
| Glycolate | 99.89 | 0.60 | 100.1 | 0.54 | No | 100.1 | 0.43 | No |
| Type A | | | | | | | | |
| (07-B3) | | | | | | | | |
| Succinate | 99.63 | 0.42 | 99.9 | 0.46 | No | 100.1 | 0.24 | No |
| Type A | | | | | | | | |
| (06-B4) | | | | | | | | |
| Mesylate | 99.09 | 0.47 | 100.1 | 0.43 | No | 100.1 | 0.44 | No |
| Type A | | | | | | | | |
| (06-B5) | | | | | | | | |

### Example 27: Preparation of Mitragynine Salts from Crude Alkaloid Extract

Crude Mitragynina speciosa alkaloid extract (810080-01-B), with weight content of mitragynine of 61.0% (75.94 area %), was used as the starting material for preparation of mitragynine salts, in order to test the ability of the different salts to separate mitragynine from a crude mixture. Two sets of experiments were set up with different charge ratios by loading different amounts of crude material and the same amount of counter ion, as follows:
(1) Treating the crude starting material as pure mitragynine free base with molar charge ratio 1:1 (Experiment ID has the prefix 810080-20-A).
(2) According to the weight purity, calculating the actual amount of mitragynine free base in crude material with molar charge ratio 1:1 (Experiment ID has the prefix 810080-20-B).

XRPD confirmed that Fumarate Type A and Mesylate Type A were obtained from charge ratio (1) and (2), L-Lactate Type A and Glycolate Type A were obtained from charge ratio (2). No L-Lactate Type A or Glycolate Type A was obtained with charge ratio (1) and no Succinate Type A was obtained with either charge ratio after slurry at RT for 3 days and then at 5° C. for 1 day. The detailed crystallization procedures are described in Table 67. All crystalline salts obtained were further characterized by ¹H NMR and HPLC, with the results summarized in Table 68.

All salts obtained were more enriched in mitragynine than the starting material, with Glycolate Type A and L-Lactate Type A having the highest mitragynine content. In contrast, only Glycolate Type A and L-Lactate Type A afforded significant purging of the corynantheidine (CR) impurity relative to the starting material.

TABLE 67
Crystallization procedures for mitragynine salts using crude alkaloid
extract (61.0 wt %, 75.94 area % mitragynine) as the starting material.

| Mitragynine Salt | | |
|---|---|---|
| (Sample ID) | Procedure | |
| Fumarate Type A | 1. | Weigh 14.9 mg of fumaric acid and 50.1 mg of crude free |
| (810080-20- | | base (810080-01-B) into a 3-mL glass vial. |
| A1_dry) | 2. | Add 1.2 mL of IPA/H₂O (4:1, v/v) to form a suspension. |
| | 3. | Magnetically stir (~1,000 rpm) at RT for 3 days. |
| | 4. | Isolate solids by centrifugation (10,000 rpm, 2 min). Dry |
| | | solids at RT under vacuum for 15.5 h. |
| Fumarate Type A | 1. | Weigh 14.9 mg of fumaric acid and 82.1 mg of crude free |
| (810080-20- | | base (810080-01-B) into a 3-mL glass vial. |
| B1_dry) | 2. | Add 1.2 mL of IPA/H₂O (4:1, v/v) to form a suspension. |
| | 3. | Magnetically stir (~1,000 rpm) at RT for 3 days. |
| | 4. | Isolate solids by centrifugation (10,000 rpm, 2 min). Dry |
| | | solids at RT under vacuum for 15.5 h. |
| L-Lactate Type A | 1. | Weigh 12.5 mg of L-lactic acid into a 3-mL glass vial. |
| (810080-20-A2) | 2. | Add 0.8 mL of IPA/H₂O (4:1, v/v) to dissolve acid. |
| | 3. | Weigh 50.0 mg of crude free base (810080-01-B) into the |
| | | acid solution. |
| | 4. | Magnetically stir (~1,000 rpm) at RT for 3 days. Clear |
| | | solution was obtained. |
| | 5. | Transferred to slurry at 5° C. for 1 day. Clear solution was |
| | | obtained. |
| L-Lactate Type A | 1. | Weigh 12.6 mg of L-lactic acid into a 3-mL glass vial. |
| (810080-20- | 2. | Add 0.8 mL of IPA/H₂O (4:1, v/v) to dissolve acid. |
| B2_dry) | 3. | Weigh 82.0 mg of crude free base (810080-01-B) into the |
| | | acid solution. |
| | 4. | Magnetically stir (~1,000 rpm) at RT for 3 days. |
| | 5. | Isolate solids by centrifugation (10,000 rpm, 2 min). Dry |
| | | solids at RT under vacuum for 15.5 h. |
| Glycolate Type A | 1. | Weigh 9.7 mg of glycolic acid and 50.2 mg of crude free |
| (810080-20-A3) | | base (810080-01-B) into a 3-mL glass vial. |
| | 2. | Add 0.8 mL of IPA/H₂O (4:1, v/v) to form a suspension. |
| | 3. | Magnetically stir (~1,000 rpm) at RT for 3 days. Clear |
| | | solution was obtained. |
| | 4. | Transferred to slurry at 5° C. for 1 day. Clear solution was |
| | | obtained. |
| Glycolate Type A | 1. | Weigh 9.8 mg of glycolic acid and 82.1 mg of crude free |
| (810080-20- | | base (810080-01-B) into a 3-mL glass vial. |
| B3_dry) | 2. | Add 0.8 mL of IPA/H₂O (4:1, v/v) to form a suspension. |
| | 3. | Magnetically stir (~1,000 rpm) at RT for 3 days. |
| | 4. | Isolate solids by centrifugation (10,000 rpm, 2 min). Dry |
| | | solids at RT under vacuum for 15.5 h. |
| Succinate Type A | 1. | Weigh 15.0 mg of succinic acid and 50.1 mg of crude free |
| (810080-20-A4) | | base (810080-01-B) into a 3-mL glass vial. |
| | 2. | Add 1.2 mL of IPA/H₂O (4:1, v/v) to form a suspension. |
| | 3. | Magnetically stir (~1,000 rpm) at RT for 3 days. Clear |
| | | solution was obtained. |
| | 4. | Transferred to slurry at 5° C. for 1 day. Clear solution was |
| | | obtained. |
| Succinate Type A | 1. | Weigh 15.1 mg of succinic acid and 81.9 mg of crude free |
| (810080-20-B4) | | base (810080-01-B) into a 3-mL glass vial. |
| | 2. | Add 1.2 mL of IPA/H₂O (4:1, v/v) to form a suspension. |
| | 3. | Magnetically stir (~1,000 rpm) at RT for 3 days. Clear |
| | | solution was obtained. |
| | 4. | Transferred to slurry at 5° C. for 1 day. Clear solution was |
| | | obtained. |
| Mesylate Type A | 1. | Weigh 12.9 mg of methanesulfonic acid into a 3-mL glass |
| (810080-20- | | vial. |
| A5_dry) | 2. | Add 1.0 mL of EtOAc to dissolve acid. |
| | 3. | Weigh 49.9 mg of crude free base (810080-01-B) into the |
| | | acid solution. |
| | 4. | Magnetically stir (~1,000 rpm) at RT for 3 days. |
| | 5. | Isolate solids by centrifugation (10,000 rpm, 2 min). Dry |
| | | solids at 50° C. under vacuum for 2 h. |
| Mesylate Type A | 1. | Weigh 12.9 mg of methanesulfonic acid into a 3-mL glass |
| (810080-20- | | vial. |
| B5_dry) | 2. | Add 1.0 mL of EtOAc to dissolve acid. |
| | 3. | Weigh 82.2 mg of crude free base (810080-01-B) into the |
| | | acid solution. |
| | 4. | Magnetically stir (~1,000 rpm) at RT for 3 days. |
| | 5. | Isolate solids by centrifugation (10,000 rpm, 2 min). Dry |
| | | solids at RT under vacuum for 15.5 h. |

TABLE 68
Characterization results of mitragynine salts obtained starting from
crude alkaloid extract (61.0 wt %, 75.94 area % mitragynine).

| Mitragynine | | | | | | |
|---|---|---|---|---|---|---|
| Salt | | | | HPLC | Weight | CR |
| (Sample ID: | Target | Charge | Obtained | purity | purity | Content |
| 810080-20) | form | ratio | form | (area %) | (%) | (area %) |
| 810080-01-B | Crude | — | — | 75.94 | 61.0 | 1.19 |
| | Alkaloid | | | | | |
| | Extract | | | | | |
| A1_dry | Fumarate | (1) | Fumarate | 87.66 | 87.25 | 1.31 |
| | Type A | | Type A | | | |
| B1_dry | Fumarate | (2) | Fumarate | 89.33 | 86.39 | 1.18 |
| | Type A | | Type A | | | |
| A2 | L-Lactate | (1) | NA | NA | NA | NA |
| | Type A | | | | | |
| B2_dry | L-Lactate | (2) | L-Lactate | 93.97 | 91.45 | 0.41 |
| | Type A | | Type A | | | |
| A3 | Gycolate | (1) | NA | NA | NA | NA |
| | Type A | | | | | |
| B3_dry | Gycolate | (2) | Gycolate | 94.45 | 88.84 | 0.36 |
| | Type A | | Type A | | | |
| A4 | Succinate | (1) | NA | NA | NA | NA |
| | Type A | | | | | |
| B4 | Succinate | (2) | NA | NA | NA | NA |
| | Type A | | | | | |
| A5_dry | Mesylate | (1) | Mesylate | 86.98 | 75.44 | 1.47 |
| | Type A | | Type A | | | |
| B5_dry | Mesylate | (2) | Mesylate | 84.51 | 75.01 | 1.39 |
| | Type A | | Type A | | | |

NA: No solid was obtained after slurry at RT for 3 days and then at 5° C. for 1 day.
(1): Treating the crude starting material as pure mitragynine free base with charge ratio 1:1.
(2): Based on weight purity, calculating the actual amount of mitragynine free base in crude material with charge ratio 1:1.

### NUMBERED EMBODIMENTS

1 A salt of 3-deuteromitragynine of Formula I

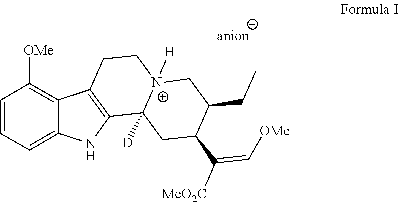 [SMILES: [2H][C@@]12C[C@H](/C(=C\OC)C(=O)OC)[C@H](CC)C[NH+]1CCc1c2[nH]c2cccc(OC)c12]

wherein the anion is glycolate, L-lactate, succinate, fumarate, or mesylate.
2 The salt of embodiment 1, wherein the anion is glycolate.
3 The salt of embodiment 1 or embodiment 2, wherein the anion is glycolate and the 3-deuteromitragynine salt exhibits an XRPD spectrum with copper radiation having 2 theta peaks at approximately 7.06. 10.12, 11.25. 15.97, 17.99, 18.11, 19.49, 19.69, 20.34, 20.88, 22.58, 25.16 and 27.60±0.2 degrees 2 theta.
4 The salt of embodiment 1, wherein the anion is L-lactate.
5 The salt of embodiment 1 or embodiment 4, wherein the anion is L-lactate and the 3-deuteromitragynine salt exhibits an XRPD spectrum with copper radiation having 2 theta peaks at approximately 6.86, 9.96, 10.73, 11.05, 15.74, 19.77, 20.63, 22.26 and 24.75±0.2 degrees 2 theta.
6 The salt of embodiment 1, wherein the anion is succinate.
7 The salt of embodiment 1 or embodiment 6, wherein the anion is succinate and the 3-deuteromitragynine salt exhibits an XRPD spectrum with copper radiation having 2 theta peaks at approximately 8.46, 10.06, 17.56, 19.27, 21.71, 23.13, 25.53, 25.94 and 31.27±0.2 degrees 2 theta.
8 The salt of embodiment 1 wherein the anion is fumarate.
9 The salt of embodiment 1 or embodiment 8 wherein the anion is fumarate and the 3-deuteromitragynine salt exhibits an XRPD spectrum with copper radiation having 2 theta peaks at approximately 8.41, 9.58, 17.46, 19.20, 23.03, 25.36, 25.77 and 31.11±0.2 degrees 2 theta.
10 The salt of embodiment 1 wherein the anion is mesylate.
11 The salt of embodiment 1 or embodiment 10, wherein the anion is mesylate and the 3-deuteromitragynine salt exhibits an XRPD spectrum with copper radiation having 2 theta peaks at approximately 6.67; 11.56, 13.34, 14.93, 16.69, 17.34, 18.60, 18.89, 20.03, 22.21, 22.71 and 25.97±0.2 degrees 2 theta.
12 A salt of mitragynine of Formula II

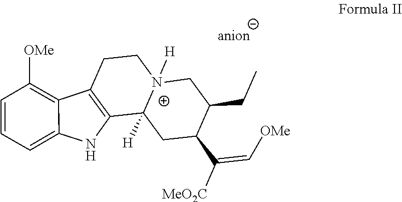 [SMILES: CC[C@@H]1C[NH+]2CCc3c([nH]c4cccc(OC)c34)[C@@H]2C[C@@H]1/C(=C\OC)C(=O)OC]

wherein the anion is glycolate, L-lactate, succinate, fumarate, or mesylate.
13 The salt of embodiment 12, wherein the anion is glycolate.
14 The salt of embodiment 12 or embodiment 13, wherein the anion is glycolate and the mitragynine salt exhibits an XRPD spectrum with copper radiation having 2 theta peaks at approximately 7.09, 10.16, 11.29, 13.23, 14.14, 15.74, 16.01, 18.03, 19.54, 19.72, 20.36, 20.93, 22.62, 25.20 and 27.63±0.2 degrees 2 theta.
15 The salt of embodiment 12, wherein the anion is L-lactate.
16 The salt of embodiment 12 or embodiment 15 wherein the anion is L-Lactate and the mitragynine salt exhibits an XRPD spectrum with copper radiation having 2 theta peaks at approximately 7.01, 10.12, 11.20, 15.57, 15.90, 17.88, 19.44, 19.91, 20.80, 22.42 and 24.90±0.2 degrees 2 theta.
17 The salt of embodiment 12, wherein the anion is succinate.
18 The salt of embodiment 12 or embodiment 17, wherein the anion is succinate and the mitragynine salt exhibits an XRPD spectrum with copper radiation having 2 theta peaks at approximately 8.47, 9.62, 14.36, 19.27, 21.71, 23.13, 25.52, 25.93 and 31.27±0.2 degrees 2 theta.
19 The salt of embodiment 12, wherein the anion is fumarate.
20 The salt of embodiment 12 or embodiment 19, wherein the anion is fumarate and the mitragynine salt exhibits an XRPD spectrum with copper radiation having 2 theta peaks at approximately 8.30, 14.29. 16.03, 16.73, 17.33, 18.55, 19.06, 19.22, 21.19. 21.44, 22.89, 23.61, 25.20, 25.62 and 28.80±0.2 degrees 2 theta.
21 The salt of embodiment 12, wherein the anion is mesylate.
22 The salt of embodiment 12 or embodiment 21, wherein the anion is mesylate and the mitragynine salt exhibits an XRPD spectrum with copper radiation having 2 theta peaks at approximately 6.71, 11.59, 13.38, 14.94, 16.71, 17.37, 18.62, 18.93, 20.07 and 25.99±0.2 degrees 2 theta.
23 A crystalline glycolate salt of 3-deuteromitragynine (3-DM), wherein the salt is glycolate Type A, glycolate Type B, glycolate Type C, glycolate Type D, glycolate Type E, glycolate Type F, or combinations thereof.
24 The Type A of 3-DM glycolate salt of embodiment 23 exhibiting an XRPD spectrum with copper radiation having 2 theta peaks at approximately 7.06, 10.11, 11.24, 15.96, 18.01, 19.49, 19.70, 20.34, 20.88, 22.57, 25.15 and 27.62±0.2 degrees 2 theta.
25 The Type B of 3-DM glycolate salt of embodiment 23 exhibiting an XRPD spectrum with copper radiation having 2 theta peaks at approximately 5.29, 5.68, 7.45, 10.78, 13.65, 19.86, 21.22, 22.72 and 24.05±0.2 degrees 2 theta.
26 The Type C of 3-DM glycolate salt of embodiment 23 exhibiting an XRPD spectrum with copper radiation having 2 theta peaks at approximately 6.03, 7.43, 10.99, 11.39, 13.65, 14.22, 16.30, 18.07, 18.84, 20.08, 21.54, 22.87, 24.16 and 26.22±0.2 degrees 2 theta.
27 The Type D of 3-DM glycolate salt of embodiment 23 exhibiting an XRPD spectrum with copper radiation having 2 theta peaks at approximately 5.33, 6.82, 9.04, 11.02, 11.33, 13.53, 13.85, 17.28, 19.50, 20.08, 21.28 and 23.71±0.2 degrees 2 theta.
28 The Type E of 3-DM glycolate salt of embodiment 23 exhibiting an XRPD spectrum with copper radiation having 2 theta peaks at approximately 5.10, 7.81, 8.82, 10.97, 12.02, 16.79, 18.40, 19.08, 20.91 and 22.25±0.2 degrees 2 theta.
29 The Type F of 3-DM glycolate salt of embodiment 23 exhibiting an XRPD spectrum with copper radiation having 2 theta peaks at approximately 5.86, 6.39, 7.21, 10.83, 11.22, 13.41, 14.05, 16.13, 17.7618.42, 19.81, 21.36, 22.07, 23.68, 23.87 and 24.38±0.2 degrees 2 theta.
30 A crystalline glycolate salt of mitragynine.
31 The crystalline mitragynine glycolate salt of embodiment 30 exhibiting an XRPD spectrum with copper radiation having 2 theta peaks at approximately 7.09, 10.16, 11.29, 13.23, 14.14, 15.74, 16.01, 18.03, 19.54, 19.72, 20.36, 20.93, 22.62, 25.20 and 27.63±0.2 degrees 2 theta and wherein the crystalline mitragynine glycolate salt is a Type A.
32 A pharmaceutical composition comprising an amount of one or more salts of 3-deuteromitragynine of Formula 1

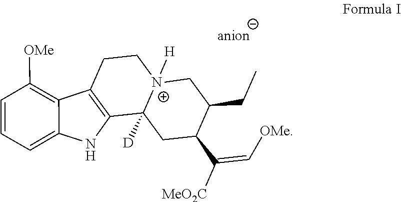 [SMILES: [2H][C@@]12C[C@H](/C(=C\OC)C(=O)OC)[C@H](CC)C[NH+]1CCc1c2[nH]c2cccc(OC)c12]

33 The pharmaceutical composition of embodiment 32, wherein the anion is glycolate, L-lactate, succinate, fumarate, or mesylate.
34 The pharmaceutical composition of embodiment 32 or embodiment 33, wherein the anion is glycolate.
35 The pharmaceutical composition of embodiment 32 or embodiment 33, wherein the anion is L-lactate.
36 The pharmaceutical composition of embodiment 32 or embodiment 33, wherein the anion is succinate.
37 The pharmaceutical composition of embodiment 32 or embodiment 33, wherein the anion is fumarate.
38 The pharmaceutical composition of embodiment 32 or embodiment 33, wherein the anion is mesylate.
39 The pharmaceutical composition of any one of embodiments 32 to 38, wherein the composition further includes a pharmaceutically acceptable carrier.
40 A pharmaceutical composition comprising an amount of one or more salts of mitragynine of Formula II

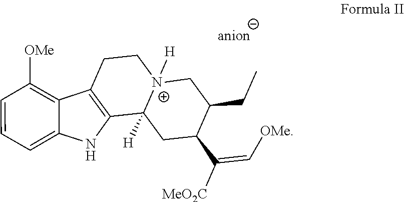 [SMILES: CC[C@@H]1C[NH+]2CCc3c([nH]c4cccc(OC)c34)[C@@H]2C[C@@H]1/C(=C\OC)C(=O)OC]

41 The pharmaceutical composition of embodiment 39-40, wherein the anion is glycolate, L-lactate, succinate, fumarate, or mesylate.
42 The pharmaceutical composition of embodiment 40 or embodiment 41, wherein the anion is glycolate.
43 The pharmaceutical composition of embodiment 40 or embodiment 41, wherein the anion is L-lactate.
44 The pharmaceutical composition of embodiment 40 or embodiment 41, wherein the anion is succinate.
45 The pharmaceutical composition of embodiment 40 or embodiment 41, wherein the anion is fumarate.
46 The pharmaceutical composition of embodiment 40 or embodiment 41, wherein the anion is mesylate.
47 The pharmaceutical composition of any one of embodiments 40 to 46, wherein the composition further includes a pharmaceutically acceptable carrier.
48 A method of treating a subject afflicted with acute pain, chronic pain, a depressive disorder, a mood disorder, an anxiety disorder, borderline personality disorder, a substance use disorder, opioid use disorder, opioid withdrawal symptoms, alcohol use disorder, or alcohol withdrawal disorder, comprising administering an effective amount of a salt or a composition as defined in any one of embodiments 1 to 47 to the subject so as to thereby treat the subject afflicted with acute pain, chronic pain, a depressive disorder, a mood disorder, an anxiety disorder, borderline personality disorder, a substance use disorder, opioid use disorder, opioid withdrawal symptoms, alcohol use disorder, or alcohol withdrawal disorder.
49 A process for producing a glycolate salt of 3-deuteromitragynine, the process including the step of crystallizing a glycolate salt of 3-deuteromitragynine from a solution of isopropyl alcohol.
50 The process of embodiment 49, wherein the solution of isopropyl alcohol includes water.
51 A process for producing a glycolate salt of mitragynine, the process including the step of crystallizing a glycolate salt of mitragynine from a solution of isopropyl alcohol.
52 The process of embodiment 51, wherein the solution of isopropyl alcohol includes water.
53 The process of embodiment 51 or embodiment 52, wherein the glycolate salt of mitragynine is derived from a crude alkaloid extract of Mitragyna speciosa.
54 A process of purifying 3-deuteromitragynine or mitragynine, the purification process including a step of crystallizing any one of the 3-deuteromitragynine or mitragynine salts as defined above in any one of embodiments 1 to 22.
55 The process of embodiment 54 wherein the resulting purified 3-deuteromitragynine or mitragynine salt is at least 90% free of other compounds or impurities.
56 The process of embodiment 55 wherein the purified 3-deuteromitragynine or mitragynine salt is at least 95% free of other compounds or impurities.
57 The process of embodiment 55 or embodiment 56 wherein the purified 3-deuteromitragynine or mitragynine salt is at least 98% free of other compounds or impurities.
58 The process of any one of embodiments 55 to 57 wherein the purified 3-deuteromitragynine or mitragynine salt is at least 99% free of other compounds or impurities.
59 The process of embodiment 54 wherein the purified 3-deuteromitragynine salt has less than about 3% of the impurity 3-deuterocorynantheidine (3-DCR).
60 The process of embodiment 59 wherein the purified 3-deuteromitragynine salt has less than about 2% of the impurity 3-DCR.
61 The process of embodiment 59 or embodiment 60 wherein the purified 3-deuteromitragynine salt has less than about 1% of the impurity 3-DCR.
62 The process of any one of embodiments 59 to 61 wherein the purified 3-deuteromitragynine salt has less than about 0.5% of the impurity 3-DCR.
63 The process of embodiment 54 wherein the purified mitragynine salt has less than about 3% of the impurity corynantheidine (CR).
64 The process of embodiment 63 wherein the purified mitragynine salt has less than about 2% of the impurity CR.
65 The process of embodiment 63 or embodiment 64 wherein the purified mitragynine salt has less than about 1% of the impurity CR.
66 The process of any one of embodiments 63 to 65 wherein the purified mitragynine salt has less than about 0.5% of the impurity CR.
67 The process of any one of embodiments 54 to 58 or embodiments 63 to 66, wherein the purified mitragynine is derived from a crude alkaloid extract of Mitragyna speciosa.
68 The process of any one of embodiments 54 to 67 wherein the purification comprises crystallizing glycolate, L-lactate, succinate, fumarate, or mesylate salt.
69 The process of any one of embodiments 54 to 68 wherein the purification comprises crystallizing the glycolate salt.

### NUMBERED EMBODIMENTS

1. A salt of 3-deuteromitragynine of Formula I:

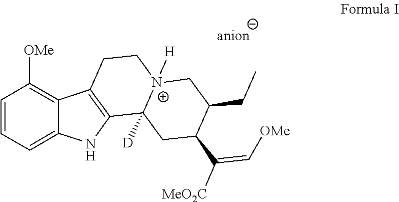 [SMILES: [2H][C@@]12C[C@H](/C(=C\OC)C(=O)OC)[C@H](CC)C[NH+]1CCc1c2[nH]c2cccc(OC)c12]

wherein the anion is glycolate, L-lactate, succinate, fumarate, or mesylate.
2. The salt of embodiment 1, wherein the anion is glycolate.
3. The salt of embodiment 2, wherein the salt of 3-deuteromitragynine of Formula I is glycolate Type A, glycolate Type B, glycolate Type C, glycolate Type D, glycolate Type E, glycolate Type F, or combinations thereof.
4. The salt of embodiment 3, wherein the salt of 3-deuteromitragynine of Formula I is glycolate Type A.
5. The salt of embodiment 4, wherein the glycolate Type A is characterized by peaks in an X-ray diffraction (XRPD) pattern at 7.1±0.2, 10.1±0.2, and 11.2±0.2° 2θ.
6. The salt of embodiment 5, wherein the glycolate Type A is further characterized by at least one XRPD peak selected from 16.0±0.2, 18.0±0.2, 19.5±0.2, 20.9±0.2, 22.6±0.2, and 25.2±0.2° 2θ.
7. The salt of any of embodiments 4-6, wherein the glycolate Type A is characterized by peaks in an XRPD pattern at 7.1±0.2, 10.1±0.2, 13.2±0.2, 16.0±0.2, 18.0±0.2, 19.5±0.2, 20.9±0.2, 22.6±0.2, and 25.2±0.2° 2θ.
8. The salt of any of embodiments 5-7, wherein the glycolate Type A is further characterized by at least one XRPD peak selected from 13.2±0.2, 14.1±0.2, 15.0±0.2, 18.5±0.2, 19.2±0.2, 19.7±0.2, 20.3±0.2, 23.7±0.2, 240±0.2, 27.6±0.2, 29.5±0.2, 30.1±0.2, 31.6±0.2, and 34.1±0.2° 2θ.
9. The salt of embodiment 4, wherein the glycolate Type A is characterized by peaks in an XRPD pattern at 7.1±0.2, 10.1±0.2, and 11.2±0.2° 2θ and at least one XRPD peak selected from 16.0±0.2, 18.0±0.2, 19.5±0.2, 19.7±0.2, 20.3±0.2, 20.9±0.2, 22.6±0.2, 25.2±0.2 and 27.6±0.2° 2θ.
10. The salt of any of embodiments 4-9, wherein the glycolate Type A is characterized by peaks in an XRPD pattern at 7.1±0.2, 10.1±0.2, 11.2±0.2, 13.2±0.2, 14.1±0.2, 15.1±0.2, 16.0±0.2, 18.0±0.2, 18.5±0.2, 19.2±0.2, 19.5±0.2, 19.7±0.2, 20.3±0.2, 20.9±0.2, 22.6±0.2, 23.7±0.2, 24.0±0.2, 25.2±0.2, 27.6±0.2, 29.5±0.2, 30.2±0.2, 31.6±0.2, and 34.1±0.2° 2θ.
11. The salt of any of embodiments 4-10, wherein the glycolate Type A exhibits a weight loss of about 1% up to a temperature of about 150° C. as measured by thermogravimetric (TGA) analysis.
12. The salt of any of embodiments 4-11, wherein the glycolate Type A exhibits a Differential Scanning calorimetry (DSC) thermogram comprising an endotherm peak at about 222±2.5° C.
13. The salt of embodiment 3, wherein the salt of 3-deuteromitragynine of Formula I is glycolate Type B.
14. The salt of embodiment 13, wherein the glycolate Type B is characterized by peaks in an XRPD pattern at 5.3±0.2, 5.7±0.2, and 7.5±0.2° 2θ.
15. The salt of embodiment 14, wherein the glycolate Type B is further characterized by at least one XRPD peak selected from 6.8±0.2, 10.8±0.2, 13.7±0.2, 19.9±0.2, 22.7±0.2, and 27.4±0.2° 2θ.
16. The salt of any of embodiments 13-15, wherein the glycolate Type B is characterized by peaks in an XRPD pattern at 5.3±0.2, 5.7±0.2, 6.8±0.2, 7.5±0.2, 10.8±0.2, 13.7±0.2, 19.9±0.2, 22.7±0.2, and 27.4±0.2° 2θ.
17. The salt of any of embodiments 14-16, wherein the glycolate Type B is further characterized by at least one XRPD peak selected from 9.0±0.2, 14.7±0.2, 17.4±0.2, 21.2±0.2, 24.1±0.2, and 25.4±0.2° 2θ.
18. The salt of embodiment 13, wherein the glycolate Type B is characterized by peaks in an XRPD pattern at 5.3±0.2, 5.7±0.2, and 7.5±0.2° 2θ and at least one XRPD peak selected from 10.8±0.2, 13.7±0.2, 19.9±0.2, 21.2±0.2, 22.7±0.2, and 24.0±0.2° 2θ.
19. The salt of any of embodiments 13-18, wherein the glycolate Type B is characterized by peaks in an XRPD pattern at 5.3±0.2, 5.7±0.2, 6.8±0.2, 7.5±0.2, 9.0±0.2, 10.8±0.2, 13.7±0.2, 14.7±0.2, 17.4±0.2, 19.9±0.2, 21.2±0.2, 22.7±0.2, 24.0±0.2, 25.4±0.2, and 27.4±0.2° 2θ.
20. The salt of any of embodiments 13-19, wherein the glycolate Type B exhibits a weight loss of about 4% up to a temperature of about 120° C. as measured by TGA analysis.
21. The salt of embodiment 20, wherein the glycolate Type B further exhibits a weight loss of about 8% between a temperature ranging from about 120° C. to about 160° C. as measured by TGA analysis.
22. The salt of any of embodiments 12-21, wherein the glycolate Type B exhibits a DSC thermogram comprising an endothermic peak at about 147±2.5° C. and 223±2.5° C.
23. The salt of embodiment 3, wherein the salt of 3-deuteromitragynine of Formula I is glycolate Type C.
24. The salt of embodiment 23, wherein the glycolate Type C is characterized by peaks in an XRPD pattern at 6.0±0.2, 7.4±0.2, and 24.2±0.2° 2θ.
25. The salt of embodiment 24, wherein the glycolate Type C is further characterized by at least one XRPD peak selected from 14.2±0.2, 16.3±0.2, 18.1±0.2, 20.1±0.2, 26.2±0.2, and 27.6±0.2° 2θ.
26. The salt of any of embodiments 23-25, wherein the glycolate Type C is characterized by peaks in an XRPD pattern at 6.0±0.2, 7.4±0.2, 14.2±0.2, 16.3±0.2, 18.1±0.2, 20.1±0.2, 24.2±0.2, 26.2±0.2, and 27.6±0.2° 2θ.
27. The salt of any of embodiments 24-26, wherein the glycolate Type C is further characterized by at least one XRPD peak selected from 10.6±0.2, 11.0±0.2, 11.4±0.2, 12.8±0.2, 13.7±0.2, 15.7±0.2, 18.8±0.2, 21.2±0.2, 21.5±0.2, 22.6±0.2, 22.9±0.2, 25.1±0.2, 28.6±0.2, 29.4±0.2, 31.6±0.2, 33.7±0.2, 35.2±0.2, and 38.3±0.2° 2θ.
28. The salt of embodiment 23, wherein the glycolate Type C is characterized by peaks in an XRPD pattern at 6.0±0.2, 7.4±0.2, and 24.2±0.2° 2θ and at least one XRPD peak at 11.0±0.2, 11.4±0.2, 13.7±0.2, 14.2±0.2, 16.3±0.2, 18.1±0.2, 18.8±0.2, 20.1±0.2, 21.5±0.2, 22.9±0.2, and 26.2±0.2° 2θ.
29. The salt of any of embodiments 23-28, wherein the glycolate Type C is characterized by peaks in an XRPD pattern at 6.0±0.2, 7.4±0.2, 10.6±0.2, 11.0±0.2, 11.4±0.2, 12.8±0.2, 13.7±0.2, 14.2±0.2, 15.7±0.2, 16.3±0.2, 17.6±0.2, 18.1±0.2, 18.8±0.2, 20.1±0.2, 21.2±0.2, 21.5±0.2, 22.6±0.2, 22.9±0.2, 24.2±0.2, 25.2±0.2, 26.2±0.2, 27.6±0.2, 28.6±0.2, 29.4±0.2, 31.6±0.2, 33.7±0.2, 35.3±0.2, and 38.3±0.2° 2θ.
30. The salt of any of embodiments 23-29, wherein the glycolate Type C exhibits a weight less of about 6% up to a temperature of about 150° C. as measured by TGA.
31. The salt of any of embodiments 23-30, wherein the glycolate Type C exhibits a DSC thermogram comprising an endotherm peak at about 61±2.5° C., 141±2.5° C., and about 222±2.5° C.
32. The salt of embodiment 3, wherein the salt of 3-deuteromitragynine of Formula I is glycolate Type D.
33. The salt of embodiment 32, wherein the glycolate Type D is characterized by peaks in an XRPD pattern at 5.3±0.2, 6.8±0.2, and 9.0±0.2° 2θ.
34. The salt of embodiment 33, wherein the glycolate Type D is further characterized by at least one XRPD peak selected from 11.0±0.2, 13.5±0.2, 17.3±0.2, 19.5±0.2, 20.1±0.2, and 21.3±0.2° 2θ.
35. The salt of any of embodiments 32-34, wherein the glycolate Type D is characterized by peaks in an XRPD pattern at 5.3±0.2, 6.8±0.2, 9.0±0.2, 11.0±0.2, 13.5±0.2, 17.3±0.2, 19.5±0.2, 20.1±0.2, and 21.3±0.2° 2θ.
36. The salt of any of embodiments 33-35, wherein the glycolate Type D is further characterized by at least one XRPD peak selected from 10.1±0.2, 11.3±0.2, 13.9±0.2, 16.5±0.2, 17.0±0.2, 18.0±0.2, 22.9±0.2, 23.7±0.2, 25.5±0.2, and 27.3±0.2° 2θ.
37. The salt of embodiment 32, wherein the glycolate Type D is characterized by peaks in an XRPD pattern at 5.3±0.2, 6.8±0.2, and 9.0±0.2° 2θ and at least one XRPD peak selected from 11.0±0.2, 11.3±0.2, 13.5±0.2, 13.9±0.2, 17.3±0.2, 19.5±0.2, 20.1±0.2, 21.3±0.2, and 23.7±0.2° 2θ.
38. The salt of any of embodiments 32-37, wherein the glycolate Type D is characterized peaks in an XRPD pattern at 5.3±0.2, 6.8±0.2, 9.0±0.2, 10.1±0.2, 11.0±0.2, 11.3±0.2, 13.5±0.2, 13.9±0.2, 16.5±0.2, 17.0±0.2, 17.3±0.2, 18.0±0.2, 19.5±0.2, 20.1±0.2, 21.3±0.2, 22.9±0.2, 23.7±0.2, 25.5±0.2, and 27.3±0.2° 2θ.
39. The salt of any of embodiments 32-38, wherein the glycolate Type D exhibits a weight less of about 3% up to a temperature of about 100° C. as measured by TGA.
40. The salt of any of embodiments 32-39, wherein the glycolate Type D exhibits a DSC thermogram comprising an endotherm peak at about 63±2.5° C., about 210±2.5° C., and about 123±2.5° C.
41. The salt of embodiment 3, wherein the salt of 3-deuteromitragynine of Formula I is glycolate Type E.
42. The salt of embodiment 41, wherein the glycolate Type E is characterized by peaks in an XRPD pattern at 5.1±0.2, 7.8±0.2, and 8.8±0.2° 2θ.
43. The salt of embodiment 42, wherein the glycolate Type E is further characterized by at least one XRPD peak selected from 11.0±0.2, 12.0±0.2, 15.1±0.2, 16.8±0.2, 19.1±0.2, and 21.0±0.2° 2θ.
44. The salt of any of embodiments 41-43, wherein the glycolate Type E is characterized by peaks in an XRPD pattern at 5.1±0.2, 7.8±0.2, 8.8±0.2, 11.0±0.2, 12.0±0.2, 15.1±0.2, 16.8±0.2, 19.1±0.2, and 21.0±0.2° 2θ.
45. The salt of any of embodiments 42-44, wherein the glycolate Type E is further characterized by at least one XRPD peak selected from 18.4±0.2 and 22.3±0.2° 2θ.
46. The salt of embodiment 41, wherein the glycolate Type E is characterized by peaks in an XRPD pattern at 5.1±0.2, 7.8±0.2, and 8.8±0.2° 2θ and at least one peak selected from 11.0±0.2, 12.0±0.2, 16.8±0.2, 18.4±0.2, 19.1±0.2, 20.9±0.2, and 22.3±0.2° 2θ.
47. The salt of any of embodiments 41-46, wherein the glycolate Type E is characterized by peaks in an XRPD pattern at 5.1±0.2, 7.8±0.2, 8.8±0.2, 11.0±0.2, 12.0±0.2, 15.1±0.2, 16.8±0.2, 18.4±0.2, 19.1±0.2, 20.9±0.2, and 22.3±0.2° 2θ.
48. The salt of embodiment 3, wherein the salt of 3-deuteromitragynine of Formula I is glycolate Type F.
49. The salt of embodiment 48, wherein the glycolate Type F is characterized by peaks in an XRPD pattern at 5.9±0.2, 6.4±0.2, and 7.2±0.2° 2θ.
50. The salt of embodiment 49, wherein the glycolate Type F is further characterized by at least one XRPD peak selected from 13.4±0.2, 14.1±0.2, 18.4±0.2, 19.8±0.2, 24.7±0.2, and 23.9±0.2° 2θ.
51. The salt of any of embodiments 48-50, wherein the glycolate Type F is characterized by peaks in an XRPD pattern at 5.9±0.2, 6.4±0.2, 7.2±0.2, 13.4±0.2, 14.1±0.2, 18.4±0.2, 19.8±0.2, 24.7±0.2, and 23.9±0.2° 2θ.
52. The salt of any of embodiments 49-51, wherein the glycolate Type F is further characterized by at least one XRPD peak selected from 10.3±0.2, 10.8±0.2, 11.2±0.2, 12.3±0.2, 16.1±0.2, 16.8±0.2, 17.2±0.2, 17.8±0.2, 20.6±0.2, 21.4±0.2, 22.1±0.2, 24.4±0.2, 26.0±0.2, 26.4±0.2, and 27.2±0.2° 2θ.
53. The salt of embodiment 48, wherein the glycolate Type F is characterized by peaks in an XRPD pattern at 5.9±0.2, 6.4±0.2, and 7.2±0.2° 2θ and at least one XRPD peak selected from 10.8±0.2, 11.2±0.2, 13.4±0.2, 14.1±0.2, 16.11±0.2, 17.8±0.2, 18.4±0.2, 19.8±0.2, 21.4±0.2, 22.1±0.2, 23.7±0.2, 23.9±0.2 and 24.4±0.2° 2θ.
54. The salt of any of embodiments 48-53, wherein the glycolate Type F is characterized by peaks in an XRPD pattern at 5.9±0.2, 6.4±0.2, 7.2±0.2, 10.3±0.2, 10.8±0.2, 11.2±0.2, 12.3±0.2, 13.4±0.2, 14.1±0.2, 16.1±0.2, 16.8±0.2, 17.2±0.2, 17.8±0.2, 18.4±0.2, 19.8±0.2, 20.6±0.2, 21.4±0.2, 22.1±0.2, 23.7±0.2, 23.9±0.2, 24.4±0.2, 26.0±0.2, 26.4±0.2, and 27.2±0.2° 2θ.
55. The salt of embodiment 1, wherein the anion is L-lactate.
56. The salt of embodiment 55, wherein the L-lactate salt is characterized by peaks in an XRPD pattern at 6.9±0.2, 10.0±0.2, and 11.0±0.2° 2θ.
57. The salt of embodiment 56, wherein the L-lactate salt is further characterized by at least one XRPD peak selected from 15.7±0.2, 20.6±0.2, 22.3±0.2, and 24.8±0.2° 2θ.
58. The salt of any of embodiments 55-57, wherein the L-lactate salt is characterized by peaks in an XRPD pattern at 6.9±0.2, 10.0±0.2, 11.0±0.2, 15.7±0.2, 20.6±0.2, 22.3±0.2, and 24.8±0.2° 2θ.
59. The salt of any of embodiments 56-58, wherein the L-lactate salt is further characterized by at least one XRPD peak selected from 10.7±0.2, 13.0±0.2, 13.8±0.2, 17.7±0.2, 18.1±0.2, 18.8±0.2, 19.3±0.2, 19.8±0.2, 23.6±0.2, 24.4±0.2, 27.0±0.2, 28.0±0.2, 29.3±0.2, 31.2±0.2, 33.8±0.2, 35.6±0.2° 2θ.
60. The salt of any of embodiments 55-59, wherein the L-lactate salt is characterized by peaks in an XRPD pattern at 6.9±0.2, 10.0±0.2, 1.07±0.2, 11.0±0.2, 13.0±0.2, 13.8±0.2, 15.8±0.2, 17.7±0.2, 18.1±0.2, 18.8±0.2, 19.3±0.2, 19.8±0.2, 20.6±0.2, 22.3±0.2, 23.6±0.2, 24.4±0.2, 24.8±0.2, 27.0±0.2, 27.1±0.2, and 35.6±0.2° 2θ.
61. The salt of any of embodiments 55-60, wherein the L-lactate salt is characterized by an XRPD pattern substantially similar to that shown in FIG. 5.
62. The salt of any of embodiments 55-61, wherein the L-lactate salt exhibits a weight less of about 1% up to a temperature of about 150° C. as measured by TGA.
63. The salt of any of embodiments 55-62, wherein the L-lactate salt exhibits a DSC thermogram comprising an endotherm peak at about 218±2.5° C.
64. The salt of embodiment 1, wherein the anion is succinate.
65. The salt of embodiment 64, wherein the succinate salt is characterized by peaks in an XRPD pattern at 8.5±0.2, 17.6±0.2, and 19.3±0.2° 2θ.
66. The salt of embodiment 65, wherein the succinate salt is further characterized by at least one XRPD peak selected 9.6±0.2, 21.7±0.2, 23.1±0.2, 25.5±0.2, and 25.9±0.2° 2θ.
67. The salt of any of embodiments 64-66, wherein the succinate salt is characterized by peaks in an XRPD pattern at 8.5±0.2, 9.6±0.2, 17.6±0.2, 19.3±0.2, 21.7±0.2, 23.1±0.2, 25.5±0.2, and 25.9±0.2° 2θ.
68. The salt of any of embodiments 65-67, wherein the succinate salt is further characterized by at least one XRPD peak selected from 6.2±0.2, 10.1±0.2, 14.4±0.2, 15.7±0.2, 16.1±0.2, 16.9±0.2, 18.7±0.2, 21.2±0.2, 22.3±0.2, 23.7±0.2, 24.5±0.2, 25.0±0.2, 25.3±0.2, 27.0±0.2, 29.1±0.2, 30.5±0.2, 33.0±0.2, and 34.3±0.2° 2θ.
69. The salt of any of embodiments 64-68, wherein the succinate salt is characterized by peaks in an XRPD pattern at 6.2±0.2, 8.5±0.2, 9.6±0.2, 10.0±0.2, 14.4±0.2, 15.7±0.2, 16.1±0.2, 16.9±0.2, 17.6±0.2, 18.7±0.2, 19.3±0.2, 21.2±0.2, 21.7±0.2, 22.3±0.2, 23.1±0.2, 23.7±0.2, 24.5±0.2, 25.0±0.2, 25.3±0.2, 25.5±0.2, 25.9±0.2, 27.0±0.2, 29.1±0.2, 30.5±0.2, 31.3±0.2, 33.0±0.2, and 34.0±0.2° 2θ.
70. The salt of any of embodiments 64-70, wherein the succinate salt is characterized by an XRPD pattern substantially similar to that shown in FIG. 7.
71. The salt of any of embodiments 64-69, wherein the succinate salt exhibits a weight less of about 2% up to a temperature of about 150° C. as measured by TGA.
72. The salt of any of embodiments 64-70, wherein the succinate salt exhibits a DSC thermogram comprising an endothermic peak at about 198±2.5° C. and about 202±2.5° C.
73. The salt of embodiment 1, wherein the anion is fumarate.
74. The salt of embodiment 73, wherein the fumarate salt is characterized by peaks in an XRPD pattern at 8.4±0.2, 17.5±0.2, and 19.2±0.2° 2θ.
75. The salt of embodiment 74, wherein the fumarate salt is further characterized by at least one XRPD peak selected from 9.6±0.2, 21.6±0.2, 25.4±0.2, 25.8±0.2, and 31.1±0.2° 2θ.
76. The salt of any of embodiments 73-75, wherein the fumarate salt is characterized by peaks in an XRPD pattern at 8.4±0.2, 9.6±0.2, 17.5±0.2, 19.2±0.2, 21.6±0.2, 25.4±0.2, 25.8±0.2, and 31.1±0.2° 2θ.
77. The salt of any of embodiments 74-76, wherein the fumarate salt is further characterized by at least one XRPD peak selected from 13.4±0.2, 14.4±0.2, 15.6±0.2, 16.2±0.2, 16.9±0.2, 18.7±0.2, 22.4±0.2, 23.0±0.2, 23.4±0.2, 23.8±0.2, 27.0±0.2, 28.9±0.2, 32.8±0.2, 34.0±0.2, and 38.0±0.2° 2θ.
78. The salt of any of embodiments 73-77, wherein the fumarate salt is characterized by peaks in an XRPD pattern at 8.4±0.2, 9.6±0.2, 13.4±0.2, 14.4±0.2, 15.6±0.2, 16.2±0.2, 16.9±0.2, 17.5±0.2, 18.7±0.2, 19.2±0.2, 21.6±0.2, 23.4±0.2, 23.0±0.2, 23.4±0.2, 23.8±0.2, 25.4±0.2, 25.8±0.2, 27.0±0.2, 28.9±0.2, 31.1±0.2, 32.8±0.2, 34.0±0.2, and 38.0±0.2° 2θ.
79. The salt of any of embodiments 73-78, wherein the fumarate salt is characterized by an XRPD pattern substantially similar to that shown in FIG. 4.
80. The salt of any of embodiments 73-79, wherein the fumarate salt exhibits a weight less of about 1% up to a temperature of about 150° C. as measured by TGA.
81. The salt of any of embodiments 73-80, wherein the fumarate salt exhibits a DSC thermogram comprising an endothermic peak at about 255±2.5° C.
82. The salt of embodiment 1, wherein the anion is mesylate.
83. The salt of embodiment 82, wherein the mesylate salt is characterized by peaks in an XRPD pattern at 6.7±0.2, 16.7±0.2, and 17.3±0.2° 2θ.
84. The salt of embodiment 83, wherein the mesylate salt is further characterized by at least one XRPD peak selected from 11.6±0.2, 13.3±0.2, 18.6±0.2, 18.9±0.2, and 20.0±0.2° 2θ.
85. The salt of any of embodiments 82-84, wherein the mesylate salt is characterized by peaks in an XRPD pattern at 6.7±0.2, 11.6±0.2, 13.3±0.2, 16.7±0.2, 17.3±0.2, 18.6±0.2, 18.9±0.2, and 20.0±0.2° 2θ.
86. The salt of any of embodiments 82-85, wherein the mesylate salt is further characterized by at least one XRPD peak selected from 8.2±0.2, 10.0±0.2, 14.9±0.2, 15.3±0.2, 19.8±0.2, 21.1±0.2, 21.4±0.2, 22.2±0.2, 22.7±0.2, 23.7±0.2, 24.4±0.2, 25.1±0.2, 26.0±0.2, 26.9±0.2, 28.5±0.2, and 32.8±0.2° 2θ.
87. The salt of any of embodiments 82-86, wherein the mesylate salt is characterized by peaks in an XRPD pattern at 6.7±0.2, 8.2±0.2, 10.0±0.2, 11.6±0.2, 13.3±0.2, 14.9±0.2, 15.3±0.2, 16.7±0.2, 17.3±0.2, 18.6±0.2, 18.9±0.2, 19.8±0.2, 20.0±0.2, 21.1±0.2, 21.4±0.2, 22.2±0.2, 22.7±0.2, 23.7±0.2, 24.4±0.2, 25.1±0.2, 26.0±0.2, 26.9±0.2, 28.5±0.2, and 32.8±0.2° 2θ.
88. The salt of any of embodiments 82-87, wherein the mesylate salt is characterized by an XRPD pattern substantially similar to that shown in FIG. 8.
89. The salt of any of embodiments 82-88, wherein the mesylate salt exhibits a weight less of about 1% up to a temperature of about 150° C. as measured by TGA.
90. The salt of any of embodiments 82-89, wherein the mesylate salt exhibits a DSC thermogram comprising an endothermic peak at about 266±2.5° C.
91. A salt of mitragynine of Formula II:

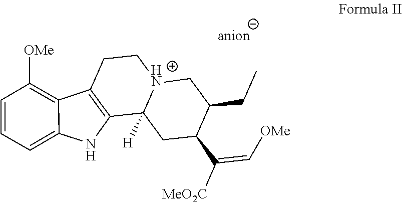

wherein the anion is glycolate, L-lactate, succinate, fumarate, or mesylate.
92. The salt of embodiment 91, wherein the anion is glycolate.
93. The salt of embodiment 92, wherein the glycolate salt is characterized by peaks in an XRPD pattern at 7.1±0.2, 10.2±0.2, and 11.3±0.2° 2θ.
94. The salt of embodiment 93, wherein the glycolate salt is further characterized by at least one XRPD peak selected from 16.0±0.2, 18.0±0.2, 19.5±0.2, 20.9±0.2, 22.6±0.2, and 25.2±0.2° 2θ.
95. The salt of any of embodiments 92-94, wherein the glycolate salt is characterized by peaks in an XRPD pattern at 7.1±0.2, 10.2±0.2, 11.3±0.2, 16.0±0.2, 18.0±0.2, 19.5±0.2, 20.9±0.2, 22.6±0.2, and 25.2±0.2° 2θ.
96. The salt of any of embodiments 93-95, wherein the glycolate salt is further characterized by at least one XRPD peak selected from 13.2±0.2, 14.1±0.2, 15.1±0.2, 15.7±0.2, 18.5±0.2, 18.9±0.2, 19.2±0.2, 19.7±0.2, 20.4±0.2, 23.3±0.2, 23.5±0.2, 23.4±0.2, 24.0±0.2, 24.9±0.2, 25.9±0.2, 27.6±0.2, 28.3±0.2, 29.1±0.2, 29.6±0.2, 30.2±0.2, 30.6±0.2, 32.7±0.2, 32.2±0.2, 34.3±0.2, 35.2±0.2, 36.0±0.2, and 36.6±0.2° 2θ.
97. The salt of any of embodiments 93-96, wherein the glycolate salt is characterized by peaks in an XRPD pattern at 7.1±0.2, 10.2±0.2, 11.3±0.2, 13.2±0.2, 14.1±0.2, 15.1±0.2, 15.7±0.2, 16.0±0.2, 18.0±0.2, 18.5±0.2, 18.9±0.2, 19.2±0.2, 19.7±0.2, 20.4±0.2, 23.3±0.2, 23.5±0.2, 23.8±0.2, 24.1±0.2, 24.9±0.2, 25.9±0.2, 27.6±0.2, 28.3±0.2, 29.1±0.2, 29.6±0.2, 30.2±0.2, 30.6±0.2, 31.7±0.2, 32.2±0.2, 34.2±0.2, 35.2±0.2, 36.0±0.2, and 36.6±0.2° 2θ.
98. The salt of any of embodiments 92-97, wherein the glycolate salt is characterized by an XRPD pattern substantially similar to that shown in FIG. 41.
99. The salt of any of embodiments 92-98, wherein the glycolate salt exhibits a weight less of about 2% up to a temperature of about 150° C. as measured by TGA.
100. The salt of any of embodiments 91-99, wherein the glycolate salt exhibits a DSC thermogram comprising an endothermic peak at about 220±2.5° C. 101. The salt of embodiment 91, wherein the anion is L-lactate.
102. The salt of embodiment 101, wherein the L-lactate salt is characterized by peaks in an XRPD pattern at 7.0±0.2, 10.1±0.2, and 11.2±0.2° 2θ.
103. The salt of embodiment 102, wherein the L-lactate salt is further characterized by at least one XRPD peak selected from 15.9±0.2, 17.9±0.2, 20.8±0.2, 22.4±0.2, and 24.9±0.2° 2θ.
104. The salt of any of embodiments 101-103, wherein the L-lactate salt is characterized by peaks in an XRPD pattern at 7.0±0.2, 10.1±0.2, 11.2±0.2, 15.9±0.2, 17.9±0.2, 20.8±0.2, 22.4±0.2, and 24.9±0.2° 2θ.
105. The salt of any of embodiments 102-104, wherein the L-lactate salt is further characterized by at least one XRPD peak selected from 10.9±0.2, 13.2±0.2, 13.9±0.2, 15.1±0.2, 15.6±0.2, 18.3±0.2, 19.0±0.2, 19.9±0.2, 21.2±0.2, 21.8±0.2, 22.9±0.2, 23.4±0.2, 23.8±0.2, 24.5±0.2, 25.8±0.2, 27.1±0.2, 27.3±0.2, 28.2±0.2, 29.5±0.2, 30.7±0.2, 31.4±0.2, 34.0±0.2, 35.7±0.2, 37.4±0.2, and 38.1±0.2° 2θ.
106. The salt of any of embodiments 101-105, wherein the L-lactate salt is characterized by peaks in an XRPD pattern at 7.0±0.2, 10.1±0.2, 10.9±0.2, 11.2±0.2, 13.2±0.2, 13.9±0.2, 15.1±0.2, 15.6±0.2, 15.9±0.2, 17.9±0.2, 18.3±0.2, 19.0±0.2, 19.9±0.2, 20.8±0.2, 21.2±0.2, 21.8±0.2, 22.4±0.2, 22.9±0.2, 23.4±0.2, 23.8±0.2, 24.5±0.2, 24.9±0.2, 25.8±0.2, 27.1±0.2, 27.3±0.2, 28.2±0.2, 29.5±0.2, 30.7±0.2, 31.4±0.2, 34.0±0.2, 35.7±0.2, 37.4±0.2, and 38.1±0.2° 2θ.
107. The salt of any of embodiments 101-106, wherein the L-lactate salt is characterized by an XRPD pattern substantially similar to that shown in FIG. 40.
108. The salt of any of embodiments 101-107, wherein the L-lactate salt exhibits a weight less of about 3% up to a temperature of about 150° C. as measured by TGA.
109. The salt of any of embodiments 101-108, wherein the L-lactate salt exhibits a DSC thermogram comprising an endothermic peak at about 226±2.5° C.
110. The salt of embodiment 91, wherein the anion is succinate.
111. The salt of embodiment 110, wherein the succinate salt is characterized by peaks in an XRPD pattern at 8.5±0.2, 17.6±0.2, and 19.3±0.2° 2θ.
112. The salt of embodiment 111, wherein the succinate salt is further characterized by at least one XRPD peak selected from 9.6±0.2, 14.4±0.2, 21.7±0.2, 23.1±0.2, 25.5±0.2, and 25.9±0.2° 2θ.
113. The salt of any of embodiments 110-112, wherein the succinate salt is characterized by peaks in an XRPD pattern at 8.5±0.2, 9.6±0.2, 14.4±0.2, 17.6±0.2, 21.7±0.2, 23.1±0.2, 25.5±0.2, and 25.9±0.2° 2θ.
114. The salt of any of embodiments 110-113, wherein the succinate salt is further characterized by at least one XRPD peak selected from 6.2±0.2, 9.1±0.2, 10.1±0.2, 13.5±0.2, 15.7±0.2, 16.1±0.2, 17.0±0.2, 18.7±0.2, 21.2±0.2, 22.3±0.2, 23.7±0.2, 24.5±0.2, 25.0±0.2, 25.3±0.2, 27.0±0.2, 28.6±0.2, 29.1±0.2, 30.5±0.2, 31.6±0.2, 33.0±0.2, 34.3±0.2, 34.6±0.2, 37.0±0.2, and 39.1±0.2° 2θ.
115. The salt of any of embodiments 110-114, wherein the succinate salt is characterized by peaks in an XRPD pattern at 6.2±0.2, 8.5±0.2, 9.1±0.2, 9.6±0.2, 10.1±0.2, 13.5±0.2, 14.4±0.2, 15.7±0.2, 16.1±0.2, 17.0±0.2, 17.6±0.2, 18.7±0.2, 21.2±0.2, 21.7±0.2, 22.3±0.2, 23.1±0.2, 23.7±0.2, 24.5±0.2, 25.0±0.2, 25.3±0.2, 25.5±0.2, 25.9±0.2, 27.0±0.2, 28.6±0.2, 29.1±0.2, 30.5±0.2, 31.6±0.2, 33.0±0.2, 34.3±0.2, 34.6±0.2, 37.0±0.2, and 39.1±0.2° 2θ.
116. The salt of any of embodiments 110-115, wherein the succinate salt is characterized by an XRPD pattern substantially similar to that shown in FIG. 42.
117. The salt of any of embodiments 110-116, wherein the succinate salt exhibits a weight less of about 4% up to a temperature of about 150° C. as measured by TGA.
118. The salt of any of embodiments 110-117, wherein the succinate salt exhibits a DSC thermogram comprising an endothermic peak at about 198±2.5° C. and about 202±2.5° C.
119. The salt of embodiment 91, wherein the anion is fumarate.
120. The salt of embodiment 119, wherein the fumarate salt is characterized by peaks in an XRPD pattern at 8.3±0.2, 19.1±0.2, and 19.2±0.2° 2θ.
121. The salt of embodiment 120, wherein the fumarate salt is further characterized by at least one XRPD peak selected from 14.3±0.2, 17.3±0.2, 18.6±0.2, 25.2±0.2, and 25.6±0.2° 2θ.
122. The salt of any of embodiments 119-121, wherein the fumarate salt is characterized by peaks in an XRPD pattern at 8.3±0.2, 14.3±0.2, 17.3±0.2, 18.6±0.2, 19.1±0.2, and 19.2±0.2, 25.2±0.2, and 25.6±0.2° 2θ.
123. The salt of any of embodiments 120-122, wherein the fumarate salt is further characterized by at least one XRPD peak selected from 9.5±0.2, 15.1±0.2, 15.5±0.2, 16.0±0.2, 16.7±0.2, 19.9±0.2, 21.2±0.2, 21.4±0.2, 22.2±0.2, 22.9±0.2, 23.2±0.2, 23.6±0.2, 24.5±0.2, 26.8±0.2, 28.8±0.2, 31.0±0.2, and 34.1±0.2° 2θ.
124. The salt of any of embodiments 119-123, wherein the fumarate salt is characterized by peaks in an XRPD pattern at 8.3±0.2, 9.5±0.2, 14.3±0.2, 15.1±0.2, 15.5±0.2, 16.0±0.2, 16.7±0.2, 17.3±0.2, 18.6±0.2, 19.1±0.2, 19.2±0.2, 19.9±0.2, 21.2±0.2, 21.4±0.2, 22.2±0.2, 22.9±0.2, 23.2±0.2, 23.6±0.2, 24.5±0.2, 25.3±0.2, 25.6±0.2, 26.8±0.2, 28.8±0.2, 31.0±0.2, and 34.1±0.2° 2θ.
125. The salt of any of embodiments 119-124, wherein the fumarate salt is characterized by an XRPD pattern substantially similar to that shown in FIG. 39.
126. The salt of any of embodiments 119-125, wherein the fumarate salt exhibits a weight less of about 3% up to a temperature of about 150° C. as measured by TGA.
127. The salt of any of embodiments 119-126, wherein the fumarate salt exhibits a DSC thermogram comprising an endothermic peak at about 226±2.5° C.
128. The salt of embodiment 91, wherein the anion is mesylate.
129. The salt of embodiment 128, wherein the mesylate salt is characterized by peaks in an XRPD pattern at 6.7±0.2, 16.7±0.2, and 17.4±0.2° 2θ.
130. The salt of embodiment 129, wherein the mesylate salt is further characterized by at least one XRPD peak selected from 11.6±0.2, 18.6±0.2, 18.9±0.2, 20.1±0.2, and 26.0±0.2° 2θ.
131. The salt of any of embodiments 128-130, wherein the mesylate salt is characterized by peaks in an XRPD pattern at 6.7±0.2, 11.6±0.2, 16.7±0.2, 17.4±0.2, 18.6±0.2, 18.9±0.2, 20.1±0.2, and 26.0±0.2° 2θ.
132. The salt of any of embodiments 129-131, wherein the mesylate salt is further characterized by at least one XRPD peak selected from 8.2±0.2, 10.0±0.2, 13.0±0.2, 13.4±0.2, 15.3±0.2, 16.4±0.2, 18.3±0.2, 19.8±0.2, 21.2±0.2, 21.4±0.2, 22.2±0.2, 22.7±0.2, 23.0±0.2, 23.7±0.2, 24.2±0.2, 24.4±0.2, 24.6±0.2, 25.1±0.2, 26.8±0.2, 27.1±0.2, 28.5±0.2, 30.1±0.2, 32.9±0.2, 33.7±0.2, and 37.1±0.2° 2θ.
133. The salt of any of embodiments 128-132, wherein the mesylate salt is characterized by peaks in an XRPD pattern at 6.7±0.2, 8.2±0.2, 10.0±0.2, 11.6±0.2, 13.0±0.2, 13.4±0.2, 15.3±0.2, 16.4±0.2, 16.7±0.2, 17.4±0.2, 18.3±0.2, 18.6±0.2, 18.9±0.2, 19.8±0.2, 20.1±0.2, 21.2±0.2, 21.4±0.2, 22.2±0.2, 22.7±0.2, 23.0±0.2, 23.7±0.2, 24.2±0.2, 24.4±0.2, 24.6±0.2, 25.1±0.2, 26.0±0.2, 26.8±0.2, 27.1±0.2, 28.5±0.2, 30.1±0.2, 32.9±0.2, 33.7±0.2, and 37.1±0.2° 2θ.
134. The salt of any of embodiments 128-133, wherein the mesylate salt is characterized by an XRPD pattern substantially similar to that shown in FIG. 43.
135. The salt of any of embodiments 128-134, wherein the mesylate salt exhibits a weight less of about 2% up to a temperature of about 150° C. as measured by TGA.
136. The salt of any of embodiments 128-135, wherein the mesylate salt exhibits a DSC thermogram comprising an endothermic peak at about 275±2.5° C.
137. A pharmaceutical composition comprising a salt of any of embodiments 1-136.
138. The pharmaceutical composition of embodiment 137 further comprising a pharmaceutically acceptable excipient.
139. A method of treating a subject afflicted with acute pain, chronic pain, a depressive disorder, a mood disorder, an anxiety disorder, borderline personality disorder, a substance use disorder, opioid use disorder, opioid withdrawal symptoms, alcohol use disorder, or alcohol withdrawal disorder, comprising administering an effective amount of a salt of any of embodiments 1-136 or a pharmaceutical composition of embodiments 137-138 to the subject.
140. The method of embodiment 139, wherein the subject is afflicted with an opioid use disorder.
141. The method of embodiment 139, wherein the subject is afflicted with opioid withdrawal.
142. The method of any one of embodiments 139-142, wherein the subject is administered about 10 mg to about 90 mg of a salt of any of embodiments 1-136.
143. The method of embodiment 142, wherein the subject is administered a glycolate salt of 3-deuteromitragynine of any one of embodiments 2-54.

1. A salt of 3-deuteromitragynine of Formula I: 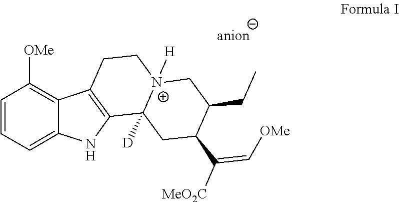 [SMILES: [2H][C@@]12C[C@H](/C(=C\OC)C(=O)OC)[C@H](CC)C[NH+]1CCc1c2[nH]c2cccc(OC)c12]
wherein the anion is glycolate, L-lactate, succinate, fumarate, or mesylate.

2. The salt of claim 1, wherein the anion is glycolate.

3. The salt of claim 2, wherein the salt of 3-deuteromitragynine of Formula I is glycolate Type A, glycolate Type B, glycolate Type C, glycolate Type D, glycolate Type E, glycolate Type F, or combinations thereof.

4. The salt of claim 3, wherein the salt of 3-deuteromitragynine of Formula I is glycolate Type A.

5. The salt of claim 4, wherein the glycolate Type A is characterized by peaks in an X-ray diffraction (XRPD) pattern at 7.1±0.2, 10.1±0.2, and 11.2±0.2° 2θ.

6. The salt of claim 5, wherein the glycolate Type A is further characterized by at least one XRPD peak selected from 16.0±0.2, 18.0±0.2, 19.5±0.2, 20.9±0.2, 22.6±0.2, and 25.2±0.2° 2θ.

7. The salt of claim 3, wherein the salt of 3-deuteromitragynine of Formula I is glycolate Type B.

8. The salt of claim 7, wherein the glycolate Type B is characterized by peaks in an XRPD pattern at 5.3±0.2, 5.7±0.2, and 7.5±0.2° 2θ.

9. The salt of claim 8, wherein the glycolate Type B is further characterized by at least one XRPD peak selected from 6.8±0.2, 10.8±0.2, 13.7±0.2, 19.9±0.2, 22.7±0.2, and 27.4±0.2° 2θ.

10. The salt of claim 3, wherein the salt of 3-deuteromitragynine of Formula I is glycolate Type C.

11. The salt of claim 10, wherein the glycolate Type C is characterized by peaks in an XRPD pattern at 6.0±0.2, 7.4±0.2, and 24.2±0.2° 2θ.

12. The salt of claim 11, wherein the glycolate Type C is further characterized by at least one XRPD peak selected from 14.2±0.2, 16.3±0.2, 18.1±0.2, 20.1±0.2, 26.2±0.2, and 27.6±0.2° 2θ.

13. The salt of claim 3, wherein the salt of 3-deuteromitragynine of Formula I is glycolate Type D.

14. The salt of claim 13, wherein the glycolate Type D is characterized by peaks in an XRPD pattern at 5.3±0.2, 6.8±0.2, and 9.0±0.2° 2θ.

15. The salt of claim 14, wherein the glycolate Type D is further characterized by at least one XRPD peak selected from 11.0±0.2, 13.5±0.2, 17.3±0.2, 19.5±0.2, 20.1±0.2, and 21.3±0.2° 2θ.

16. The salt of claim 3, wherein the salt of 3-deuteromitragynine of Formula I is glycolate Type E.

17. The salt of claim 16, wherein the glycolate Type E is characterized by peaks in an XRPD pattern at 5.1±0.2, 7.8±0.2, and 8.8±0.2° 2θ.

18. The salt of claim 17, wherein the glycolate Type E is further characterized by at least one XRPD peak selected from 11.0±0.2, 12.0±0.2, 15.1±0.2, 16.8±0.2, 19.1±0.2, and 21.0±0.2° 2θ.

19. The salt of claim 3, wherein the salt of 3-deuteromitragynine of Formula I is glycolate Type F.

20. The salt of claim 19, wherein the glycolate Type F is characterized by peaks in an XRPD pattern at 5.9±0.2, 6.4±0.2, and 7.2±0.2° 2θ.

21. The salt of claim 20, wherein the glycolate Type F is further characterized by at least one XRPD peak selected from 13.4±0.2, 14.1±0.2, 18.4±0.2, 19.8±0.2, 24.7±0.2, and 23.9±0.2° 2θ.

22. The salt of claim 1, wherein the anion is L-lactate.

23. The salt of claim 22, wherein the L-lactate salt is characterized by peaks in an XRPD pattern at 6.9±0.2, 10.0±0.2, and 11.0±0.2° 2θ.

24. The salt of claim 23, wherein the L-lactate salt is further characterized by at least one XRPD peak selected from 15.7±0.2, 20.6±0.2, 22.3±0.2, and 24.8±0.2° 2θ.

25. The salt of claim 1, wherein the anion is succinate.

26. The salt of claim 25, wherein the succinate salt is characterized by peaks in an XRPD pattern at 8.5±0.2, 17.6±0.2, and 19.3±0.2° 2θ.

27. The salt of claim 26, wherein the succinate salt is further characterized by at least one XRPD peak selected 9.6±0.2, 21.7±0.2, 23.1±0.2, 25.5±0.2, and 25.9±0.2° 2θ.

28. The salt of claim 1, wherein the anion is fumarate.

29. The salt of claim 28, wherein the fumarate salt is characterized by peaks in an XRPD pattern at 8.4±0.2, 17.5±0.2, and 19.2±0.2° 2θ.

30. The salt of claim 29, wherein the fumarate salt is further characterized by at least one XRPD peak selected from 9.6±0.2, 21.6±0.2, 25.4±0.2, 25.8±0.2, and 31.1±0.2° 2θ.

31. The salt of claim 1, wherein the anion is mesylate.

32. The salt of claim 31, wherein the mesylate salt is characterized by peaks in an XRPD pattern at 6.7±0.2, 16.7±0.2, and 17.3±0.2° 2θ.

33. The salt of claim 32, wherein the mesylate salt is further characterized by at least one XRPD peak selected from 11.6±0.2, 13.3±0.2, 18.6±0.2, 18.9±0.2, and 20.0±0.2° 2θ.

34. A salt of mitragynine of Formula II 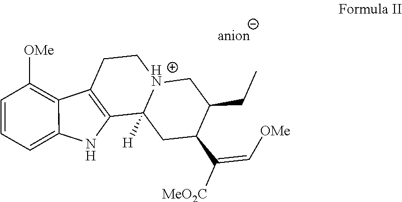
wherein the anion is glycolate, L-lactate, succinate, fumarate, or mesylate.

35. The salt of claim 34, wherein the anion is glycolate.

36. The salt of claim 35, wherein the glycolate salt is characterized by peaks in an XRPD pattern at 7.1±0.2, 10.2±0.2, and 11.3±0.2° 2θ.

37. The salt of claim 36, wherein the glycolate salt is further characterized by at least one XRPD peak selected from 16.0±0.2, 18.0±0.2, 19.5±0.2, 20.9±0.2, 22.6±0.2, and 25.2±0.2° 2θ.

38. The salt of claim 34, wherein the anion is L-lactate.

39. The salt of claim 38, wherein the L-lactate salt is characterized by peaks in an XRPD pattern at 7.0±0.2, 10.1±0.2, and 11.2±0.2° 2θ.

40. The salt of claim 39, wherein the L-lactate salt is further characterized by at least one XRPD peak selected from 15.9±0.2, 17.9±0.2, 20.8±0.2, 22.4±0.2, and 24.9±0.2° 2θ.

41. The salt of claim 34, wherein the anion is succinate.

42. The salt of claim 41, wherein the succinate salt is characterized by peaks in an XRPD pattern at 8.5±0.2, 17.6±0.2, and 19.3±0.2° 2θ.

43. The salt of claim 42, wherein the succinate salt is further characterized by at least one XRPD peak selected from 9.6±0.2, 14.4±0.2, 21.7±0.2, 23.1±0.2, 25.5±0.2, and 25.9±0.2° 2θ.

44. The salt of claim 34, wherein the anion is fumarate.

45. The salt of claim 44, wherein the fumarate salt is characterized by peaks in an XRPD pattern at 8.3±0.2, 19.1±0.2, and 19.2±0.2° 2θ.

46. The salt of claim 45, wherein the fumarate salt is further characterized by at least one XRPD peak selected from 14.3±0.2, 17.3±0.2, 18.6±0.2, 25.2±0.2, and 25.6±0.2° 2θ.

47. The salt of claim 34, wherein the anion is mesylate.

48. The salt of claim 47, wherein the mesylate salt is characterized by peaks in an XRPD pattern at 6.7±0.2, 16.7±0.2, and 17.4±0.2° 2θ.

49. The salt of claim 48, wherein the mesylate salt is further characterized by at least one XRPD peak selected from 11.6±0.2, 18.6±0.2, 18.9±0.2, 20.1±0.2, and 26.0±0.2° 2θ.

50. A pharmaceutical composition comprising a salt of claim 1.

51. The pharmaceutical composition of claim 50, further comprising a pharmaceutically acceptable excipient.

52. A method of treating a subject afflicted with acute pain, chronic pain, a depressive disorder, a mood disorder, an anxiety disorder, borderline personality disorder, a substance use disorder, opioid use disorder, opioid withdrawal symptoms, alcohol use disorder, or alcohol withdrawal disorder, comprising administering an effective amount of a salt of claim 1 and a pharmaceutically acceptable excipient to the subject.

53. The method of claim 52, wherein the subject is afflicted with an opioid use disorder.

54. The method of claim 52, wherein the subject is afflicted with opioid withdrawal.
